(19) United States
(12) Patent Application Publication
(10) Pub. No.: US 2006/0252981 A1
(43) Pub. Date: Nov. 9, 2006

(54) Biocompatible implant and use of the same

(76) Inventors: Hikaru Matsuda, Hyogo (JP); Yoshiki Sawa, Hyogo (JP); Satoshi Taketani, Taketani (JP); Shigemitsu Iwai, Osaka (JP); Koichiro Hirakawa, Hanagawa (JP)

Correspondence Address: BRINKS HOFER GILSON & LIONE, P.O. BOX 10395, CHICAGO, IL, 60610, US

(21) Appl. No.: 10/537,678

(22) PCT Filed: Dec. 5, 2003
(86) PCT No.: PCT/JP03/15641

§ 371 Date: Mar. 14, 2006

(30) Foreign Application Priority Data
Dec. 5, 2002 (JP) 2002-354342
Sep. 11, 2003 (JP) 2003-320491

(51) Int. Cl. A61F 2/02; C12N 5/08

(52) U.S. Cl. 600/37; 435/401; 623/23.76

(57) ABSTRACT

The present invention provides an implant capable of being cellularized in treatment of an injured organ or tissue in organisms. The present inventors found that a biocompatible implant comprising a biological molecule and a support is capable of being cellularized. The implant can be used instead of conventional implants which essentially comprise cells. The present invention provides a biocompatible implant comprising A) a biological molecule and B) a support. The present invention also provides A) a first layer having a rough surface, B) a rough surface; B) a second layer having a strength which allows the support to resist in vivo shock. The first layer is attached to the second layer via at least one point.

### TECHNICAL FIELD

The present invention relates to a biocompatible implant, a method for producing or using the implant, and a medicament and treatment method relevant thereto. Hereinafter, the present invention will be described in detail.

### BACKGROUND ART

Implantation of organs (e.g., heart, blood vessel, etc.) derived from exogenous tissue is mainly hindered by immunological rejections. Changes occurring in allografts and xenografts were first described 90 or more years ago (Carrel A., 1907, J. Exp. Med. 9:226-8; Carrel A., 1912., J. Exp. Med. 9:389-92; Calne R. Y., 1970, Transplant Proc. 2:550; and Auchincloss 1988, Transplantation 46:1). Rejection to artery grafts pathologically leads either to enlargement (up to rupture) or obstruction of the grafts. The former is caused by decomposition of extracellular matrices, while the latter is caused by proliferation of cells in a blood vessel (Uretsky B. F., Mulari S., Reddy S., et al., 1987, Circulation 76:827-34).

Conventionally, two strategies have been used to alleviate rejection of these substances. One of the two strategies is to reduce the immune reaction of hosts (Schmitz-Rixen T., Megerman J., Colvin R. B., Williams A. M., Abbot W., 1988, J. Vasc. Surg. 7:82-92; and Plissonnier D., et al., 1993, Arteriosclerosis Thromb, 13:112-9). The other is to reduce the antigenicity of allografts or xenografts mainly by cross-linking (Rosenberg N., et al., 1956, Surg. Forum 6:242-6; and Dumont C., Pissonnier D., Michel J. B., 1993, J. Surg. Res. 54:61-69). The cross-linking of extracellular matrices reduces the antigenicity of grafts, but changes bioengineering functions (Cosgrove D. M., Lytle B. W., Golding C. C., et al., 1983, J. Thorac. Cardiovasc. Surgery 64:172-176; and Broom N., Christie G. W., 1982, In: Cohn L. H., Gallucci V., editors. Cardiac bioprostheses: Proceedings of the Second International Symposium. New York: York Medical Books Pages 476-491), so that the grafts become susceptible to mineralization (Schoen F. J., Levy R. J., Piehler H. R., 1992, Cardiovasc. Pathology 1992; 1:29-52).

Conventionally, heterologous pericardium or self pericardium treated with glutaraldehyde has been used as a cardiovascular repair patch. However, this patch has problems to be solved, such as calcification, thrombus formation, hyper susceptibility to infection, low durability, and the like. To solve these problems, higher biocompatible cardiovascular repair artificial patches (Tissue Engineered Bioprosthetic Patch) are being developed using tissue engineering.

Implantation of a graft coated with cells has been tried (Shunji Niioka, Yasuharu Imai, Kazuhiro Seo, et al., “TissuEnjiniaringu niyoru ShinkekkanZairyo no Kaihatu, Oyo [Development and Application of Cardiovascular Material by Tissue Engineering], Journal of the Japanese Society for Cardiovascular Surgery, 2000, 29, 38; and J. Thorac. Cardiovasc. Surg., 1998; 115; 536-46). Unfortunately, grafts are not satisfactorily coated with cells; use of cells has immunological disadvantages; and the like. Therefore, there is a keen demand for a support (e.g., an artificial patch) which is easy to produce and handle and has substantially no immunological problems. There are problems with cell coating, cell collection methods, sites for cell collection, immunological matter, infection during ex vivo culture, facility environment, or the like. Therefore, there is a keen demand for a support (e.g., an artificial patch) which is easy to produce and handle and has substantially no immunological problems.

It is considered to be preferable that a tissue or organ repaired by transplantation is cellularized, i.e., the tissue or organ behaves as if it is self tissue or self organ (e.g., growth after transplantation, etc.), in order to treat tissue or organ injury. No technique for modifying a tissue or organ for in-situ cellularization has been achieved.

It is known that biocompatible materials are provided in the form of knits or wovens, for example. Non-biocompatible materials in the form of knits have been reported to be successfully used as artificial tissues. However, most knit biocompatible materials are insufficient in terms of strength or the like. In addition, the knit form has a structural drawback in that it is likely to permit liquid to leak. Thus, there has been no knit material which is successfully employed in vivo in the shape of a support (e.g., a patch).

Biocompatible materials in the form of wovens are also often used. The woven form is superior in terms of strength. However, wovens inevitably become frayed and cannot be necessarily said to be suitable for in vivo use.

To date, no implant or support usable for biocompatible patches or the like have been available.

Japanese Laid-Open Publication No. 2002-543950 discloses a biological macromolecule material containing a particulate reinforcing medium, however, it is not intended to be implanted into organisms. This material comprises a biological adhesive for performing adhesion by crosslinking albumin with aldehyde. The adhesive is sandwiched by a reinforcing agent. However, the regeneration of tissue is not intended by this material. The residual aldehyde may be harmful.

Japanese Laid-Open Publication No. 2001-78750 discloses a scaffold for cells which consists of a foam member and a reinforcing member, however, implantation into organisms is not an intendable. Particularly, this arrangement has a drawback in that its physical properties are specified by materials. This publication describes that cells are seeded before implantation. Thus, the object of the technique described in the publication is considered to provide a scaffold for in vitro culture. A support for regeneration is not intended by the technique.

International Publication WO89/05371 discloses a scaffold for cells, however, it does not describe implantation of the scaffold into organisms for reinforcement and regeneration of organs.

Therefore, an object of the present invention is to provide an implant capable of being cellularized and a support for use in the implant for the treatment of injuries in biological organs or tissues.

### DISCLOSURE OF THE INVENTION

The present inventors have rigorously studied and unexpectedly found a biocompatible implant comprising a biological molecule and a support, which is capable of being cellularized. This implant can be used instead of conventional implants which essentially comprise cells. Thus, the above-described problems can be solved by the present invention. The present inventors also found a support comprising A) a first layer having a rough surface: and B) a second layer having a strength which allows the support to resist in vivo shock, in which the first layer is attached to the second layer, which can be used unexpectedly for tissue regeneration. This support has a high level of durability and biological affinity and a sufficient level of strength. Thereby, the above-described problems can be solved.

The present invention also provides a structure comprising biocompatible knit and woven implant layers and an intermediate layer for attaching the knit layer with the woven layer. This structure can unexpectedly solve both the leakage problem with knit and the fray problem with woven. The combination of knit and woven also unexpectedly provides a material which has space for accommodating cells while preventing leakage and fray. In addition, by providing a biological molecule (e.g., collagens, cytokines, chemokines, etc.) to the support, when the support is placed in organisms, cells aggregate to the support in the early period and subsequently the support itself is biologically degraded and eventually vanishes. Thereby, a graft which leaves substantially no trace can be provided. By selecting any method to produce knit and woven, the composite support is given a predetermined strength and a predetermined thickness. The absorption rates of knit and woven can be controlled by selecting any materials for threads used in the knit and the woven. Further, a support suited to the regeneration rate of a tissue and having a required strength can be produced. Thus, the present invention is considered to be used in various applications. In an embodiment of the present invention, a woven is used. The physical properties of a woven are not specified by a material constituting it and can be regulated by changing a weaving manner. Thus, the above-described conventional drawback can be circumvented. The strength of a woven can also be made to a predetermined level or more by changing a weaving manner. In addition, when a woven is used, it is possible to easily select a material whose degradation rate can be regulated more simply to freely produce various supports. Thus, the present invention can provide more various supports as compared to conventional technology.

Therefore, the present invention provides the following.

(1) A biocompatible implant, comprising:

A) a biological molecule: and

B) a support.

(2) A biocompatible implant according to item 1, wherein the biological molecule includes a protein.

(3) A biocompatible implant according to item 1, wherein the biological molecule includes a cellular physiologically active substance.

(4) A biocompatible implant according to item 1, wherein the biological molecule includes a cell adhesion molecule.

(5) A biocompatible implant according to item 1, wherein the biological molecule includes an extracellular matrix.

(6) A biocompatible implant according to item 1, wherein the biological molecule includes a cellular adhesive protein.

(7) A biocompatible implant according to item 1, wherein the biological molecule includes an integrin.

(8) A biocompatible implant according to item 1, wherein the biological molecule is selected from the group consisting of collagen and laminin.

(9) A biocompatible implant according to item 1, wherein the biological molecule includes a fiber forming collagen or basement membrane collagen.

(10) A biocompatible implant according to item 1, wherein the biological molecule includes a fiber forming collagen and basement membrane collagen.

(11) A biocompatible implant according to item 1, wherein the biological molecule includes type I collagen or type IV collagen.

(12) A biocompatible implant according to item 1, wherein the biological molecule includes collagen and cytokine.

(13) A biocompatible implant according to item 1, wherein the support is in the form of a membrane.

(14) A biocompatible implant according to item 1, wherein the support is in the form of a tube.

(15) A biocompatible implant according to item 1, wherein the support is in the form of a valve.

(16) A biocompatible implant according to item 1, wherein the support includes biodegradable polymer.

(17) A biocompatible implant according to item 1, wherein the support includes at least one component selected from the group consisting of poly(glycolic acid) (PGA), poly(L-lactic acid) (PLA) and polycaprolactum (PCLA).

(18) A biocompatible implant according to item 1, wherein the support includes PGLA having a glycolic acid-to-lactic acid ratio of from about 90: about 10 to about 80: about 20.

(19) A biocompatible implant according to item 1, wherein the support includes a cell adhesion molecule.

(20) A biocompatible implant according to item 1, wherein the support includes a protein.

(21) A biocompatible implant according to item 1, wherein the support is in the form of a mesh and a sponge.

(22) A biocompatible implant according to item 1, wherein the support has a thickness of at least about 0.2 mm to about 1.0 mm.

(23) A biocompatible implant according to item 1, wherein the support has a strength of at least about 20 N.

(24) A biocompatible implant according to item 1, wherein the support has a strength of at least about 50 N.

(25) A biocompatible implant according to item 1, wherein the support is coated with the biological molecule.

(26) A biocompatible implant according to item 1, wherein the support has a gap and the gap is filled with the biological molecule.

(27) A biocompatible implant according to item 1, wherein the biological molecule and the support include a crosslinking molecule, and the crosslinking molecules are crosslinked between the support and the biological molecule.

(28) A biocompatible implant according to item 1, wherein the support includes the same material as the biological molecule.

(29) A biocompatible implant according to item 1, wherein a cell is attached to the biocompatible implant.

(30) A biocompatible implant according to item 1, for use in implantation into a body.

(31) A biocompatible implant according to item 30, wherein a site of the body into which the biological implant is implanted is selected from the group consisting of cardiac valve, blood vessel, pericardium, cardiac septum, intracardiac conduit, extracardiac conduit, dura mater, skin, bone, soft tissue and trachea.

(32) A biocompatible implant according to item 1, which is sterilized.

(33) A biocompatible implant according to item 1, further comprising an immunosuppressant.

(34) A biocompatible implant according to item 1, further comprising an additional medicament component.

(35) A biocompatible implant according to item 30, wherein the biocompatible implant is derived from an organism undergoing the implantation.

(36) A medicament according to item 1, comprising a biocompatible implant according to item 1.

(37) A medical kit, comprising:

a biocompatible implant according to item 1; and

instructions describing usage of the implant,

wherein the instructions describe that the implant is administered to a predetermined site.

(38) A medical kit according to item 37, wherein the predetermined site is selected from the group consisting of vascular endothelium, vascular smooth muscle, elastic fiber, skeletal muscle, cardiac muscle, osteoblast, neuron and collagen fiber.

(39) A medical kit according to item 37, wherein the instructions describe that the biocompatible implant is implanted in such a manner that at least a part of an organ or tissue to be subjected to implantation is left in situ.

(40) A method for treating an injured site of a body, comprising the step of:

A) implanting a biocompatible implant to a part or whole of the injured site,

wherein the biocompatible implant comprises:

A-1) a biological molecule; and

A-2) a support.

(41) A method according to item 40, wherein in the implanting step, the biocompatible implant is implanted in such a manner that at least a part of an organ or tissue to which the injured site belongs is left in situ.

(42) A method according to item 40, further comprising administering a cellular physiologically active substance.

(43) A method according to item 42, wherein the cellular physiologically active substance is selected from the group consisting of a granulocyte macrophage colony stimulating factor (GM-CSF), a macrophage colony stimulating factor (M-CSF), a granulocyte colony stimulating factors (G-CSF), a multi-CSF (IL-3), a leukemia inhibiting factor (LIF), a c-kit ligand (SCF), an immunoglobulin family member, CD2, CD4, CD8, CD44, collagen, elastin, proteoglycan, glycosaminoglycan, fibronectin, laminin, syndecan, aggrecan, an integrin family member, integrin a chain, integrin β chain, fibronectin, laminin, vitronectin, selectin, cadherin, ICM1, ICAM2, VCAM1, platelet derived growth factor (PDGF), epidermal growth factor (EGF), fibroblast growth factor (FGF), hepatocyte growth factor (HGF) and vascular endothelial growth factor (VEGF), and polypeptides and peptides related thereto.

(44) A method according to item 40, further comprising performing a treatment for suppressing an immune reaction.

(45) A method for reinforcing an organ or tissue in a body, comprising the step of:

A) implanting a biocompatible implant to a part or whole of the organ or tissue,

wherein the biocompatible implant comprises:

A-1) a biological molecule; and

A-2) a support.

(46) A method for producing or regenerating an organ or tissue, comprising the steps of:

A) implanting a biocompatible implant to a part or whole of the organ or tissue within an organism containing the organ or tissue,

wherein the biocompatible implant comprises:

A-1) a biological molecule; and

A-2) a support; and

B) culturing the organ or tissue within the organism.

(47) Use of a biocompatible implant according to item 1 for treatment of an injured site within a body.

(48) Use of a biocompatible implant according to item 1 for reinforcement of an organ or tissue within a body.

(49) Use of a biocompatible implant according to item 1 for production of a medicament for treatment of an injured site within a body.

(50) Use of a biocompatible implant according to item 1 for production of a medicament for reinforcement of an organ or tissue within a body.

(51) A biocompatible tissue support, comprising:

A) a first layer having a rough surface; and

B) a second layer having a strength which allows the second layer to resist in vivo impact,

wherein the first layer is attached to the second layer via at least one point.

(52) A support according to item 51, wherein the first layer is a knit.

(53) A support according to item 51, wherein the second layer is a woven.

(54) A support according to item 51, wherein the rough surface has sufficient space for accommodating cells.

(55) A support according to item 51, wherein the attachment is carried out by melting a biological absorbable macromolecule.

(56) A support according to item 51, wherein the second layer has substantially no permeability to air.

(57) A support according to item 51, wherein the strength of the support is at least 100 N.

(58) A support according to item 51, wherein the air permeability of the support is no more than 10 ml/cm²/sec.

(59) A support according to item 51, wherein the first layer includes a biodegradable material.

(60) A support according to item 51, wherein the first layer includes at least one component selected from the group consisting of poly(glycolic acid) (PGA), poly(L-lactic acid) (PLA), and polycaprolactum (PCLA) and a copolymer thereof.

(61) A support according to item 51, wherein the first layer includes PGLA having a glycolic acid-to-lactic acid ratio of from about 90: about 10 to about 80: about 20.

(62) A support according to item 51, wherein the first layer includes poly(glycolic acid).

(63) A support according to item 51, wherein the second layer includes a biodegradable material.

(64) A support according to item 51, wherein the second layer includes at least one component selected from the group consisting of poly(glycolic acid) (PGA), poly(L-lactic acid)(PLA) and polycaprolactum (PCLA), and a copolymer thereof.

(65) A support according to item 51, wherein the second layer includes PGLA having a glycolic acid-to-lactic acid ratio of from about 90: about 10 to about 80: about 20.

(66) A support according to item 51, wherein the second layer includes poly(L-lactic acid).

(67) A support according to item 51, wherein the second layer is a woven and the first layer is a knit.

(68) A support according to item 51, wherein the second layer is a woven of poly(L-lactic acid) and the first layer is a knit of poly(glycolic acid).

(69) A support according to item 51, wherein the attachment is carried out by:

C) an intermediate layer for attaching the first layer with the second layer.

(70) A support according to item 69, wherein the intermediate layer is made of a synthetic biological absorbable polymer.

(71) A support according to item 69, wherein the intermediate layer includes a homopolymer containing a single monomer selected from the group consisting of lactic acid (lactid), glycolide and ε-caprolactam or a copolymer containing two or more monomers therefrom.

(72) A support according to item 69, wherein the intermediate layer includes a material having a melting point lower than a melting point of the second layer and a melting point of the first layer.

(73) A support according to item 51, wherein the first layer comprises a plurality of knit layers.

(74) A support according to item 51, wherein the first layer comprises a plurality of knit layers.

(75) A support according to item 51, wherein a biological molecule is provided on the first layer.

(76) A support according to item 75, wherein the biological molecule is an extracellular matrix.

(77) A support according to item 75, wherein the biological molecule includes an extracellular matrix selected from the group consisting of collagen and laminin.

(78) A support according to item 75, wherein the biological molecule is contained in a microsponge and the microsponge is provided on the first layer.

(79) A support according to item 75, wherein the biological molecule is crosslinked with the support.

(80) A medical device comprising a support according to item 51.

(81) A medical device according to item 80, further comprising a cell.

(82) A medicament according to item 80, for use in implantation into a body.

(83) A medicament according to item 80, wherein a site of the body into which the biological implant is implanted is selected from the group consisting of cardiac valve, blood vessel, pericardium, cardiac septum, intracardiac conduit, extracardiac conduit, dura mater, skin, bone, soft tissue and trachea.

(84) A medicament according to item 80, wherein the biocompatible implant is derived from an organism undergoing the implantation.

(85) A method for producing a biocompatible tissue support, wherein the biocompatible tissue support comprises:

A) a first layer having a rough surface; and

B) a second layer having a strength which allows the second layer to resist in vivo impact,

wherein the first layer is attached to the second layer via at least one point, and

the method comprises the step of:
attaching the first layer with the second layer.
   (86) A method according to item 85, wherein the biocompatible tissue support further comprises:

C) an intermediate layer for attaching the first layer with the second layer,

the attaching step comprises:
a) providing the intermediate layer between the first layer and the second layer;
b) providing the first layer, the second layer and the intermediate layer under conditions that the first layer and the second layer are not melted and the intermediate layer is melted; and
c) the intermediate layer is provided under conditions that the intermediate layer is solidified, while retaining desired shapes of the first layer, the second layer and the intermediate layer.
   (87) A method according to item 86, wherein the melting point of the intermediate layer is lower than both the melting points of the first layer and the second layer and a difference between the melting points is utilized.
   (88) A method according to item 86, wherein the second layer is a woven of poly(L-lactic acid) and the first layer is a kit of poly(glycolic acid), and the intermediate layer includes a homopolymer containing a single monomer selected from the group consisting of lactic acid (lactid), glycolide and ε-caprolactam or a copolymer containing two or more monomers therefrom.
   (89) A method according item 88, wherein the temperature is higher than the melting point of the intermediate layer and is lower than the melting points of the first layer and the second layer.
   (90) A method according to item 86, wherein the support further comprises a biological molecule and the method further comprises the step of:

attaching the biological molecule to the first layer.

(91) A method according to item 90, wherein the attaching step comprises crosslinking treatment.

(92) A method according to item 90, wherein the biological molecule is collagen, and the attaching step comprises collagen crosslinking treatment.

(93) A method according to item 86, wherein the intermediate layer is produced by casting a film material onto a glass plate, followed by air drying, to form a film.

(94) A method according to item 86, wherein the step b) comprises exerting a pressure of at least about 0.1 g/cm² onto the support.

(95) A method according to item 86, wherein the step b) comprises exerting a pressure of at least about 0.5 g/cm² onto the support.

(96) A method for treating an injured site of a body, comprising the step of:

A) implanting a biocompatible tissue support to a part or whole of the injured site,

wherein the biocompatible tissue support comprises:

A-1) a first layer having a rough surface; and

A-2) a second layer having a strength which allows the second layer to resist in vivo impact,

wherein the first layer is attached to the second layer via at least one point.

(97) A method for reinforcing an organ or tissue within a body, comprising the step of:

A) implanting a biocompatible tissue support to a part or whole of the injured site,

wherein the biocompatible tissue support comprises:

A-1) a first layer having a rough surface; and

A-2) a second layer having a strength which allows the second layer to resist in vivo impact,

wherein the first layer is attached to the second layer via at least one point.

(98) A method for producing or regenerating an organ or tissue, comprising the steps of:

A) implanting a biocompatible tissue support to a part or whole of the organ or tissue within an organism containing the organ or tissue,

wherein the biocompatible tissue support comprises:

A-1) a first layer having a rough surface; and

A-2) a second layer having a strength which allows the second layer to resist in vivo impact,

wherein the first layer is attached to the second layer via at least one point; and

B) culturing the organ or tissue in the organism.

(99) Use of a biocompatible tissue support for treatment of an injured site within a body, wherein

the biocompatible tissue support comprises:

A-1) a first layer having a rough surface; and

A-2) a second layer having a strength which allows the second layer to resist in vivo impact,

wherein the first layer is attached to the second layer via at least one point.

(100) Use of a biocompatible tissue support for reinforcement of an organ or tissue within a body, wherein

the biocompatible tissue support comprises:

A-1) a first layer having a rough surface; and

A-2) a second layer having a strength which allows the second layer to resist in vivo impact,

wherein the first layer is attached to the second layer via at least one point.

(101) Use of a biocompatible tissue support for production of a medicament for treatment of an injured site within a body, wherein

the biocompatible tissue support comprises:

A-1) a first layer having a rough surface; and

A-2) a second layer having a strength which allows the second layer to resist in vivo impact,

wherein the first layer is attached to the second layer via at least one point.

(102) Use of a biocompatible tissue support for production of a medicament for reinforcement of an organ or tissue within a body, wherein

the biocompatible tissue support comprises:

A-1) a first layer having a rough surface: and

A-2) a second layer having a strength which allows the second layer to resist in vivo impact,

wherein the first layer is attached to the second layer via at least one point.

According to the present invention, an implant capable of being cellularized is provided without using a self-reproducing biological material, such as a cell or the like. Conventionally, organ or tissue regeneration has never been observed by implanting such an implant. Thus, the present invention achieves an unexpected effect. In addition, the present invention also provides a biocompatible support which overcomes drawbacks of conventional knits and wovens.

Hereinafter, the present invention will be described by way of preferred embodiments. It will be understood by those skilled in the art that the embodiments of the present invention can be appropriately made or carried out based on the description of the present specification and commonly used technique well known in the art. The function and effect of the present invention can be easily recognized by those skilled in the art.

### BRIEF DESCRIPTION OF THE DRAWINGS

FIG. 1 shows an exemplary biocompatible implant according to the present invention.

FIG. 2 is a photograph showing a state of implantation.

FIG. 3 is a graph showing a mechanical strength.

FIG. 4 is a diagram showing an in vitro cell adhesion efficiency.

FIG. 5 is a diagram showing an in vivo state two weeks after implantation.

FIG. 6 is a diagram showing an in vivo state two months after implantation.

FIG. 7 is a diagram showing a state of vascular endothelial cells two months after implantation.

FIG. 8 is a diagram showing a state of vascular smooth muscle cells two months after implantation.

FIG. 9 is a diagram showing a state of elastic fiber cells two months after implantation.

FIG. 10 is a diagram showing an in vivo state six months after implantation.

FIG. 11 is another diagram showing an in vivo state six months after implantation.

FIG. 12 is a diagram showing a state of calcification six months after implantation.

FIG. 13A is a photograph of a poly(glycolic acid) mesh taken from the top side thereof.

FIG. 13B is a photograph of a poly(L-lactic acid) mesh taken from the top side thereof.

FIG. 14 is a photograph showing a state of a poly(glycolic acid) knit taken from the bottom side thereof.

FIG. 15 is a photograph showing a state of a poly(glycolic acid) knit taken from the top side thereof.

FIG. 16A is a cross-sectional view showing a poly(glycolic acid) knit, from which it is observed that loops are continuously joined.

FIG. 16B is a cross-sectional view showing a poly(glycolic acid) knit and a poly(L-lactic acid) woven.

FIG. 17 is a schematic diagram showing a method for attaching a poly(glycolic acid) knit with a poly(L-lactic acid) woven.

FIG. 18 shows a method for producing a support according to the present invention.

FIG. 19 is a schematic diagram showing collagen crosslinking.

FIG. 20A shows an exemplary support (poly(glycolic acid)) crosslinked with collagen.

FIG. 20B shows an exemplary support (poly(L-lactic acid)) crosslinked with collagen.

FIG. 21 shows various exemplary supports crosslinked with collagen.

FIG. 22A shows the tensile strength of various supports (poly(glycolic acid)).

FIG. 22B shows the tensile strength of various supports (poly(L-lactic acid)).

FIG. 23 shows the modulus of elasticity of various supports.

FIG. 24 shows the strain of various supports.

FIG. 25 shows the water leakage rate of various supports.

FIG. 26 shows the air permeability of various supports.

FIG. 27A shows in vitro cellular adhesiveness (poly(glycolic acid)).

FIG. 27B shows in vitro cellular adhesiveness (poly(L-lactic acid)).

FIG. 28 shows an exemplary test protocol for an adhesion condition study test.

FIG. 29A shows the results of an adhesion condition test.

FIG. 29B shows the result of an attachment condition test (polycaprolactum concentration) for a composite support of the present invention.

FIG. 29C shows the result of an attachment condition (pressure) test for a composite support of the present invention.

FIG. 29D shows the result of an attachment condition (temperature and time) test for a composite support of the present invention.

FIG. 30 shows a surface shape as a result of a strength deterioration test (Week 0, 1, 3 and 6).

FIG. 31 shows a result of a strength deterioration test, indicating a change in weight (A), a change in maximum point load (B), and a change in rate of a maximum point load (C).

FIG. 32 is a schematic diagram showing a procedure for implanting a support into a rat heart according to the present invention.

FIG. 33 shows a state of a rat heart one month after infarction in the case of no support implantation.

FIG. 34 shows a state one month after implantation of a support (without a biological molecule) according to the present invention.

FIG. 35 shows a state one month after implantation of a support (with type IV collagen and type I) according to the present invention.

FIG. 36 shows an example of implantation into a rat myocardial infarction site.

FIG. 37 shows an example of implantation into a rat myocardial infarction site (only a composite material).

FIG. 38 shows an example of implantation into a rat myocardial infarction site (a composite material coated with type I collagen, type IV collagen and laminin).

FIG. 39 shows the assessment of cardiac function in implantation into a rat myocardial infarction site.

FIG. 40 shows an example of implantation into the dorsum of a rat.

FIG. 41 shows an example of implantation into the dorsum of a rat (a PLGA material coated with type I collagen and HGF).

FIG. 42 shows an example of implantation into the dorsum of a rat (a composite material coated with type I collagen and HGF).

FIG. 43 shows the result of real-time PCR for an example of implantation into the dorsum of a rat (a composite material coated with type I collagen and HGF).

FIG. 44 shows an example of implantation into the dorsum of a rat (a PLGA material coated with type I collagen, type IV collagen and laminin).

FIG. 45 shows an example of implantation into the dorsum of a rat (a composite material coated with type I collagen, type IV collagen and laminin).

FIG. 46 shows the level of expression of each cell marker in an example of implantation into the dorsum of a rat (a composite material coated with type I collagen, type IV collagen and laminin).

FIG. 47 shows the result of a cell growth test (vascular endothelial cell) for a composite support of the present invention.

FIG. 48 shows the result of a cell growth test (vascular smooth muscle cell) for a composite support of the present invention.

FIG. 49 shows the protocol and the result of a fray test for a composite support of the present invention.

FIG. 50 shows the result of implantation of a composite support of the present invention (two months). The upper left portion shows the result of smooth muscle actin (SMA) staining of the aorta two months after implantation of poly(glycolic acid) (knit)+poly(L-lactic acid) (woven) (×100 magnification). The upper right portion shows the result of Factor VIII staining of the aorta two months after implantation of poly(glycolic acid) (knit)+poly(L-lactic acid) (woven) (×100 magnification). The lower left portion shows the result of smooth muscle actin (SMA) staining of the pulmonary artery two months after implantation of poly(glycolic acid) (knit)+poly(L-lactic acid) (woven) (×100 magnification). The lower right portion shows the result of Factor VIII staining of the pulmonary artery two months after implantation of poly(glycolic acid) (knit)+poly(L-lactic acid) (woven) (×100 magnification).

FIG. 51 shows the result of implantation of a PLGA support of the present invention (two months). The lower left portion shows the result of Factor VIII staining of the pulmonary artery two months after implantation of a PLGA copolymer (porous material) (×100 magnification). The lower right portion shows the result of smooth muscle actin (SMA) staining of the pulmonary artery two months after implantation of a PLGA copolymer (porous material) (×100 magnification).

FIG. 52 shows an exemplary support with a monocusp of the present invention (one-monocusp support).

FIG. 53 shows another exemplary support with a monocusp of the present invention (one-monocusp support). A biodegradable scaffold reinforced with woven poly-lactic acid mesh crosslinking with collagen-microsponge was formed into a transannular patch with monocusp.

FIG. 54 shows that the attachment and proliferation of seeding cells cultured on the PLGA-collagen-microsponge was significantly higher than the PLGA with simple collagen-coat and PLGA only (*: p<0.05 vs PLGA only, **: p<0.05 vs PLGA-collagen-coat and PLGA only).

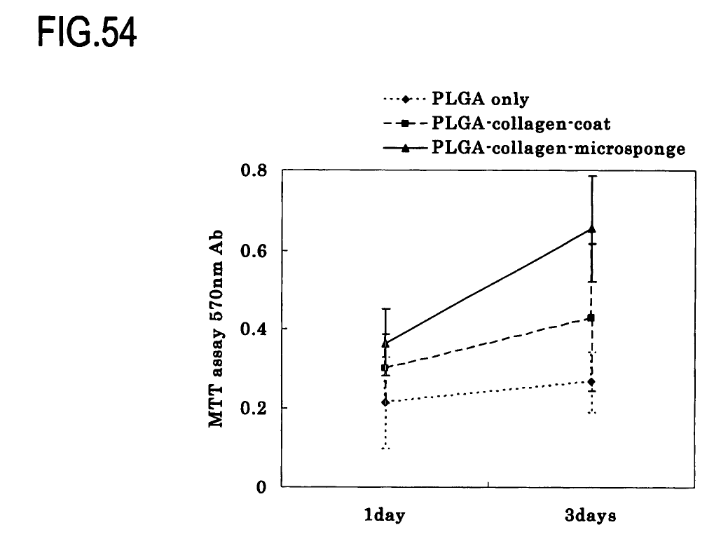

FIG. 55 shows a state of an implanted support with a monocusp of the present invention (monocusp support).

FIG. 56 shows that the present invention actually works in a transannular patch model. In the transannular patch model, transesophageal echocardiography (TEE) and angiography two months after grafting showed good leaflet function and no pulmonary regurgitation. The arrow shows prosthetic leaf let. LA: left atrium, RV: right ventricle, PA: pulmonary artery, RVG(L): lateral view of RV-graphy, PAG(L): lateral view of PA-graphy. FIG. 56 show sequential photographs which demonstrate that a monocusp support of the present invention functions as an actual cusp, taken by echocardiography. As can be seen from these figures, the valve shown in the middle of the photograph was opened and was then closed.

### DESCRIPTION OF SEQUENCE LISTING

SEQ ID NO:1 indicates the amino acid sequence of a short peptide used In Example 19.

SEQ ID NO:2 indicates the nucleic acid sequence of a 5′ primer for identification of cardiac action.

SEQ ID NO:3 indicates the nucleic acid sequence of a 3′ primer for identification of cardiac action.

SEQ ID NO:4 indicates the nucleic acid sequence of a probe for identification of cardiac action.

SEQ ID NO:5 indicates the nucleic acid sequence of a 5′ primer for identification of α-MHC.

SEQ ID NO:6 indicates the nucleic acid sequence of a 3′ primer for identification of α-MHC.

SEQ ID NO:7 indicates the nucleic acid sequence of a probe for identification of α-MHC.

SEQ ID NO:8 indicates the nucleic acid sequence of a 5′ primer for identification of β-MHC.

SEQ ID NO:9 indicates the nucleic acid sequence of a 3′ primer for identification of β-MHC.

SEQ ID NO:10 indicates the nucleic acid sequence of a probe for identification of β-MHC.

### BEST MODE FOR CARRYING OUT THE INVENTION

It should be understood throughout the present specification that singular forms include plural referents unless the context clearly dictates otherwise. It should be also understood that the terms as used herein have definitions typically used in the art unless otherwise mentioned.

The definitions of terms used herein are described below.

As used herein, the term “regeneration” refers to a phenomenon in which when an individual organism loses, or congenitally lacks, a portion of tissue, the remaining tissue grows and recovers voluntarily or with help of another material. As used herein, the term “regeneration” also indicates that cells or the like aggregate an injured tissue or organ within an organism and the cells are multiplied or amplitude. The extent or manner of regeneration varies depending among animal species or among tissues in the same individual. Most human tissues have limited regeneration capability, and therefore, complete regeneration is not expected if a large portion of tissue is lost. In the case of severe damage, tissue having strong proliferation capability different from that of lost tissue may grow, resulting in incomplete regeneration where the damaged tissue is incompletely regenerated and the function of the tissue cannot be recovered. In this case, a structure made of a bioabsorbable material is used to prevent tissue having a strong proliferation capability from infiltrating the defective portion of the tissue so as to secure space for proliferation of the damaged tissue. Further, by supplementing with a cell growth factor, the regeneration capability of the damaged tissue is enhanced. Such a regeneration technique is applied to cartilages, bones, and peripheral nerves, for example. It has been so far believed that nerve cells and cardiac muscles have no or poor regeneration capability. Recently, it was reported that there are tissue stem cells (somatic stem cells) which have both the capability of differentiating into these tissues and self-proliferation capability. Expectations are running high for regenerative medicine using tissue stem cells. Embryonic stem cells (ES cells) are cells which have the capability of differentiating into all tissues. Efforts have been made to use ES cells for regeneration of complicated organs, such as kidney, liver, and the like, but have not yet been realized. Thus, a regeneration method for introducing stem cells into a tissue is an attractive method. Therefore, an implant of the present invention may include such a stem cell.

As used herein, the term “cellularized” or “in-situ cellularization” in relation to implantation means that an implanted implant functions as a part of an organ or tissue of a host. Therefore, the term “in-situ cellularization” indicates, but is not limited to, that an implant acquires a self-reproducing ability; material or device components voluntarily aggregate to form a structure without any help of a human, or components voluntarily forms a pattern in the course of a dynamic process in which energy or material is dissipated (compatibility with surrounding tissue, minimization of reaction with foreign matter (inflammation, endosporial growth, hardening, calcification); and possibility of growth). Whether or not a graft or implant is cellularized can be herein determined by using a marker for confirming the growth of a self cell, such as a von Willebrand factor, α-SMA, elastica van Gieson for elastic tissue, or the like.

Specifically, whether or not a graft is cellularized can be determined by histological search of cell pattern formation and self arrangement; the presence or absence of an immune reaction; measurement of electrical connection as accurate synthesis of cell aggregation; measurement of functions by an ultrasonic test; hydroproline assay; elastin assay; DNA assay; quantification of the number of cells; quantification of protein; glycosaminoglycan assay; and myosin heavy chain assay, though the present invention is not limited to these. For example, in the case of a blood vessel, whether or not an implant is cellularized can be determined by determining the presence or absence of the new formation of a blood vessel. The number of blood vessels can be determined by immunohistochemically staining blood vessels with a Factor VIII-relevant antigen or the like and counting the stained blood vessels. Specifically, specimens are fixed with 10% buffered formalin, followed by paraffin embedding. Several continuous slices are prepared from each specimen, followed by freezing. Next, the frozen slice is fixed with 2% paraformaldehyde in PBS for 5 min at room temperature and is immersed in methanol containing 3% hydrogen peroxide for 15 min, followed by washing with PBS. This sample is covered with bovine serum albumin solution for about 10 min to block non-specific reactions. The specimen is coupled with HRP, followed by incubation overnight with an EPOS-conjugated antibody for the VIII-relevant antigen. After the sample is washed with PBS, the sample is immersed in diaminobenzidine solution (e.g., 0.3 mg/ml diaminobenzidine in PBS) to obtain positive staining. Stained vascular endothelial cells are counted under, for example, an optical microscope (×100 magnification). For example, the result of counting is represented by the number of blood vessels per square millimeters. After a specific treatment, it is determined whether or not the number of blood vessels increased statistically significantly, so as to confirm the presence of Factor VIII. Thereby, for example, the presence and angiogenesis activity of vascular endothelial cells can be determined. Preferably, whether or not an implant is cellularized is determined by measurement of potential of an aggregation of cells as an accurate synthesis by a patch clamp method, i.e., electrophysiological measurement, such as electricity density analysis for determining whether or not an electrophysiological activity is the same as that of a host. If such an electrical connection is present, such a state is also herein referred to “electrically cellularized”.

As used herein, the term “biological molecule” refers to a molecule relating to an organism and an aggregation thereof. As used herein, the term “biological” or “organism” refers to a biological organism, including, but being not limited to, an animal, a plant, a fungus, a virus, and the like. A biological molecule includes a molecule extracted from an organism and an aggregation thereof, though the present invention is not limited to this. Any molecule capable of affecting an organism and an aggregation thereof fall within the definition of a biological molecule. Therefore, low molecular weight molecules (e.g., low molecular weight molecule ligands, etc.) capable of being used as medicaments fall within the definition of biological molecule as long as an effect on an organism is intended. Examples of such a biological molecule include, but are not limited to, a protein, a polypeptide, an oligopeptide, a peptide, a polynucleotide, an oligonucleotide, a nucleotide, a nucleic acid (e.g., DNA such as cDNA and genomic DNA; RNA such as mRNA), a polysaccharide, an oligosaccharide, a lipid, a low molecular weight molecule (e.g., a hormone, a ligand, an information transmitting substance, a low molecular weight organic molecule, etc.), and a composite molecule thereof and an aggregation thereof (e.g., an extracellular matrix, a fiber, etc.), and the like. In the present invention, a biological molecule is preferably compatible, or may be adapted to be compatible, with a host in need of implantation. It can be determined whether or not a certain biological molecule is compatible, or may be adapted to be compatible, with a host, as follows. The biological molecule is implanted into the host. A side reaction, such as immune rejection reaction or the like, is suppressed, if required. It is observed and determined whether or not the biological molecule is stable in the host. An example of a preferable biological molecule for use in the present invention includes, for example, a biological molecule having affinity with a cell, such as an extracellular matrix. In another preferred embodiment of the present invention, a biological molecule capable of inducing a cell may be used.

The terms “protein”, “polypeptide”, “oligopeptide” and “peptide” as used herein have the same meaning and refer to an amino acid polymer having any length or variants thereof. This polymer may be a straight, branched or cyclic chain. An amino acid may be a naturally-occurring or nonnaturally-occurring amino acid, or a variant amino acid. The term may include those assembled into a complex of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example. When used in an implant of the present invention, a “protein” is preferably compatible with a host in need of the implant, though any protein may be used as long as the protein can be adapted to be compatible with the host. It is determined whether or not a certain protein is compatible, or may be adapted to be compatible, with a host as follows. The protein is implanted into the host. A side reaction, such as immune rejection reaction or the like, is suppressed, if required. It is observed and determined whether or not the protein is stable in the host. Representatively, an example of such a compatible protein includes, but is not limited to, a protein derived from a host.

As used herein, the term “cellular physiologically active substance” refers to a substance capable of acting on a cell or tissue. Examples of such an action include, but are not limited to, control, change, and the like of the cell or tissue. Cellular physiologically active substances include cytokines and growth factors. A cellular physiologically active substance may be naturally-occurring or synthesized. Preferably, a cellular physiologically active substance is one that is produced by a cell or one that has a function similar thereto. As used herein, a cellular physiologically active substance may be in the form of a protein or a nucleic acid or in other forms. In actual practice, cellular physiologically active substances are typically proteins.

The term “cytokine” is used herein in the broadest sense in the art and refers to a physiologically active substance which is produced from a cell and acts on the same or different cell. Cytokines are generally proteins or polypeptides having a function of controlling an immune response, regulating the endocrine system, regulating the nervous system, acting against a tumor, acting against a virus, regulating cell growth, regulating cell differentiation, or the like. Cytokines are herein in the form of a protein or a nucleic acid or in other forms. In actual practice, cytokines are typically proteins.

The terms “growth factor” or “cell growth factor” are used herein interchangeably and each refers to a substance which promotes or controls cell growth. Growth factors are also called “proliferation factor” or “development factor”. Growth factors may be added to cell or tissue culture medium, substituting for serum macromolecules. It has been revealed that a number of growth factors have a function of controlling differentiation in addition to a function of promoting cell growth.

Examples of cytokines representatively include, but are not limited to, interleukins, chemokines, hematopoietic factors such as colony stimulating factors, a tumor necrosis factor, interferons, a platelet-derived growth factor (PDGF), an epidermal growth factor (EGF), a fibroblast growth factor (FGF), a hepatocyte growth factor (HGF), an endothelial cell growth factor (VEGF), cardiotrophin, and the like, which have proliferative activity.

Cellular physiologically active substances, such as cytokines, growth factors, and the like, typically have redundancy in function. Accordingly, reference herein to a particular cytokine or growth factor by one name or function also includes any other names or functions (e.g., cell adhesion activity, cell-substrate adhesion activity, etc.) by which the factor is known to those of skill in the art, as long as the factor has the activity of a cellular physiologically active substance for use in the present invention. Cytokines or growth factors can be used in a preferred embodiment of the present invention as long as they have preferable activity (e.g., activity to aggregate host cells, etc.) as described herein.

As used herein, the term “extracellular matrix” (ECM) refers to a substance existing between somatic cells no matter whether the cells are epithelial cells or non-epithelial cells. Extracellular matrices are involved in supporting tissue as well as in internal environmental structure essential for survival of all somatic cells. Extracellular matrices are generally produced from connective tissue cells. Some extracellular matrices are secreted from cells possessing basal membrane, such as epithelial cells or endothelial cells. Extracellular matrices are roughly divided into fibrous components and matrices filling there between. Fibrous components include collagen fibers and elastic fibers. A basic component of matrices is a glycosaminoglycan (acidic mucopolysaccharide), most of which is bound to non-collagenous protein to form a polymer of a proteoglycan (acidic mucopolysaccharide-protein complex). In addition, matrices include glycoproteins, such as laminin of basal membrane, microfibrils around elastic fibers, fibers, fibronectins on cell surfaces, and the like. Particularly differentiated tissue has the same basic structure. For example, in hyaline cartilage, chondroblasts characteristically produce a large amount of cartilage matrices including proteoglycans. In bones, osteoblasts produce bone matrices which cause calcification. Examples of an extracellular matrix for use in the present invention include, but are not limited to, collagen, elastin, proteoglycan, glycosaminoglycan, fibronectin, laminin, elastic fiber, collagen fiber, and the like. When used in the present invention, the extracellular matrix preferably has activity to aggregate self cells of a host.

As used herein, the terms “cell adhesion molecule” and “adhesion molecule” are used interchangeably, referring to a molecule capable of mediating the joining of two or more cells (cell adhesion) or adhesion between a substrate and a cell. In general, cell adhesion molecules are divided into two groups: molecules involved in cell-cell adhesion (intercellular adhesion) (cell-cell adhesion molecules) and molecules involved in cell-extracellular matrix adhesion (cell-substrate adhesion) (cell-substrate adhesion molecules). For an implant of the present invention, either type of molecule is useful and can be effectively used. Therefore, cell adhesion molecules herein include a protein of a substrate and a protein of a cell (e.g., integrin, etc.) in cell-substrate adhesion. A molecule other than proteins falls within the concept of cell adhesion molecule as long as it can mediate cell adhesion.

For cell-cell adhesion, cadherin, a number of molecules belonging in an immunoglobulin superfamily (NCAM, L1, ICAM, fasciclin II, III, etc.), selectin, and the like are known, each of which is known to join cell membranes via a specific molecular reaction.

On the other hand, a major cell adhesion molecule functioning for cell-substrate adhesion is integrin, which recognizes and binds to various proteins contained in extracellular matrices. These cell adhesion molecules are all located on cell membranes and can be regarded as a type of receptor (cell adhesion receptor). Therefore, receptors present on cell membranes can also be used in an implant of the present invention. Examples of such a receptor include, but are not limited to, myalpha α-integrin, β-integrin, CD44, syndecan, aggrecan, and the like.

Note that extracellular matrix molecules (cellular adhesive protein, such as fibronectin, laminin, and the like), which are bound by integrin or the like, herein fall within the category of cell adhesion molecules. A function shared by each adhesion receptor in cell-cell adhesion and cell-substrate adhesion is not strictly defined and varies depending on the distribution of binding molecules (ligand). For example, a certain integrin is involved in cell-cell adhesion, such as hemocyte-hemocyte adhesion or the like. It is known that when a growth factor, cytokine or the like is present as a cell membrane protein, a reaction with its receptor present on other cells eventually causes cell adhesion. Such a growth factor or cytokine can be used as a biological molecule contained in an implant of the present invention.

Thus, various molecules are involved in cell adhesion and have different functions. Those skilled in the art can appropriately select a molecule to be contained in an implant of the present invention depending on the purpose. Techniques for cell adhesion are well known as described above and as described in, for example, “Saibogaimatorikkusu-Rinsho heno Oyo-[Extracellular matrix-Clinical Applications-], Medical Review.

It can be determined whether or not a certain molecule is a cell adhesion molecule, by an assay, such as biochemical quantification (an SDS-PAG method, a labeled-collagen method, etc.), immunological quantification (an enzyme antibody method, a fluorescent antibody method, an immunohistological study, etc.), a PDR method, a hybridization method, or the like, in which a positive reaction is detected. Examples of such a cell adhesion molecule include, but are not limited to, collagen, integrin, fibronectin, laminin, vitronectin, fibrinogen, an immunoglobulin superfamily member (e.g., CD2, CD4, CD8, ICM1, ICAM2, VCAM1), selectin, cadherin, and the like. Most of these cell adhesion molecules transmit into a cell an auxiliary signal for cell activation due to intercellular interaction as well as cell adhesion. Therefore, an adhesion factor for use in an implant of the present invention preferably transmits an auxiliary signal for cell activation into a cell. This is because cell activation can promote growth of cells originally present or aggregating in a tissue or organ at an injured site after application of an implant thereto. It can be determined whether or not such an auxiliary signal can be transmitted into a cell, by an assay, such as biochemical quantification (an SDS-PAG method, a labeled-collagen method, etc.), immunological quantification (an enzyme antibody method, a fluorescent antibody method, an immunohistological study, etc.), a PDR method, a hybridization method, or the like, in which a positive reaction is detected.

An example of a cell adhesion molecule is cadherin which is present in many cells capable of being fixed to tissue. Cadherin can be used in a preferred embodiment of the present invention. Examples of a cell adhesion molecule in cells of blood and the immune system which are not fixed to tissue, include, but are not limited to, immunoglobulin superfamily molecules (CD 2, LFA-3, ICAM-1, CD2, CD4, CD8, ICM1, ICAM2, VCAM1, etc.); integrin family molecules (LFA-1, Mac-1, gpIIbIIIa, p150, p95, VLA1, VLA2, VLA3, VLA4, VLA5, VLA6, etc.); selectin family molecules (L-selectin, E-selectin, P-selectin, etc.), and the like. Therefore, such a molecule may be useful for treatment of a tissue or organ of blood and the immune system.

Nonfixed cells need to be adhered to a specific tissue in order to act on the tissue. In this case, it is believed that cell-cell adhesion is gradually enhanced via a first adhesion by a selectin molecule or the like which is constantly expressed and a second adhesion by a subsequently activated integrin molecule. Therefore, in the present invention, a cell adhesion molecule for mediating the first adhesion and another cell adhesion molecule for mediating the second adhesion may be used together.

As used herein, the term cellular adhesive protein” refers to a protein capable of mediating cell adhesion as described above. Therefore, as used herein, the term “cellular adhesive protein” includes a protein (e.g., integrin, etc.) of a cell as well as a protein of a substrate in cell-substrate adhesion. For example, when cultured cells are seeded on a substrate (glass or plastic) adsorbing a protein under serum-free conditions, a receptor integrin recognizes the cellular adhesive protein and adheres to the substrate. An active site of a cellular adhesive protein has been determined at the amino acid level. As such an active site, RGD, YIGSR or the like are known (these are collectively called “RGD sequences”). Therefore, in one preferred embodiment, a protein contianed in an implant of the present invention may advantageously has an RGD sequence, such as RGD, YIGSR, or the like. Typically, a cellular adhesive protein is present in an extracellular matrix, the surface of a cultured cell, and body fluid (plasma, serum, etc.). It is known that the in vivo function of cellular adhesive proteins include migration, growth, morphological regulation, tissue construction and the like of cells as well as adhesion of cells to an extracellular matrix. In addition to action on cells, some proteins are capable of regulating blood coagulation and complement action. Such proteins may be useful in the present invention. Examples of such a cellular adhesive protein include, but are not is limited to, fibronectin, collagen, vitronectin, laminin, and the like.

As used herein, the term “RGD molecule” refers to a protein molecule comprising an amino acid sequence RGD (Arg-Gly-Asp) or a sequence having the same function as that of the sequence RGD. RGD molecules are characterized by comprising an amino acid sequence RGD which is useful as an amino acid sequence of a cell adhesion active site of a cellular adhesive protein or another amino acid sequence having an equivalent function. The RGD sequence was found as a cell adhesion site of fibronectin, and subsequently, a number of molecules having cellular adhesive activity were found, including collagentype I, laminin, vitronectin, fibrinogen, the von Willebrand factor, entactin, and the like. If a chemically synthesized RGD peptide is attached to a solid phase, the peptide exhibits cell adhesion activity. A biological molecule of the present invention may be a chemically synthesized RGD molecule. Examples of such an RGD molecule include, but are not limited to, a GRGDSP peptide in addition to the above-described naturally-occurring molecules. The RGD sequence is recognized by integrin (e.g., a receptor for fibronectin) which is a cell adhesion molecule (and also a receptor). Therefore, a molecule having a function equivalent to RGD can be identified by examining interaction with integrin.

As used herein, the term “integrin” refers to a transmembrane glycoprotein which is a receptor involved in cell adhesion. Integlins are located on cell surfaces and function when a cell adheres to an extracellular matrix. It is known that integrins are involved in cell-cell adhesion in the hemocyte system. Examples of such integrins include, but are not limited to, receptors for fibronectin, vitronectin, collagen, or the like: IIb/IIIa in platelets: Mac-1 in macrophages; LFA-1, VLA-1 to 6 in lymphocytes; PSA in fruit flies; and the like. Typically, integrins have a hetero dimer structure in which an α chain having a molecular weight of 130 kDa to 210 kDa and a β chain having a molecular weight of 95 kDa to 130 kDa are associated via a non-covalent bond. Examples of the α chain include, but are not limited to, α¹, α², α³, α⁴, α⁵, α⁶, α^{L}, α^{M}, α^{X}, α^{IIb}, α^{V}, α^{E}, and the like. Examples of the β chain include, but are not limited to, β₁, β₂, β₃, β₄, β₅, β₆, β₇, and the like.

Examples of such a hetero dimer include, but are not limited to, Gp IIb IIIa, VLA-1, VLA-2, VLA-3, VLA-4, VLA-5, VLA-6, CD51/CD29, LFA-1, Mac-1, p150, p90, a vitronectin receptor, β⁴ subfamily, β⁵ subfamily, β⁶ subfamily, LPAM-1, HML-1, and the like. Typically, it is often that the extracellular domain of the α chain has a divalent cation binding site, and the extracellular domain of the β chain has a cysteine-rich domain and the intracellular domain of the β chain has a tyrosine phosphorylation site. A recognition site of a binding ligand is often the RGD sequence. Therefore, integrin may be an RGD molecule.

As used herein, the term “collagen” is a generic term referring to a type of protein which is fiber forming collagen in which three polypeptide chains forms a triple helix, and which functions as a scaffold for cell acceptance and growth and forms the tissue skeleton. Collagen is a major component of an animal extracellular matrix. It is known that collagen also has the RGD sequence and exhibits cell adhesion activity. Collagen is known to account for 20 to 30% of all proteins of an animal and to be contained particularly in skin, tendon, cartilage, and the like in large quantities. Collagen molecules of type I to XIII are known. Typically, a collagen molecule has a triple helix structure consisting of three polypeptide chains. Each chain is often called an α chain. A collagen molecule may consist of a single type of α chain or a plurality of types of α chains encoded by different genes. α chains are typically designated by α plus a suffix like α¹, α², α³, and so on, and another suffix indicating collagen type may be further added like α1(I) and so on. Therefore, in the present invention, for example, a naturally-occurring collagen molecule [α1(I)₂α2(I)] (type I collagen) and a trimer consisting of nonnaturally-occurring chains may be used. Most parts of the primary structure of collagen have an amino acid sequence of [Gly-X-Pro(or hydroxyprolyl)]ₙ(X: any amino acid residue). This structure has a left-handed helix structure where three residues form one pitch. Collagen typically contains hydroxylysine as a specific amino acid. Collagen is a glycoprotein in which a sugar portion is coupled with a hydroxyl group of hydroxylysine.

There are two types of collagen: fiber forming collagen which aggregate to form collagen fiber or interstitial collagen. There are fiber forming collagens of type I, type II, type III, type V, and type XI, which are used in a preferred embodiment of the present invention. In addition, collagen includes short-chain collagen (type VIII, type X, etc.), basement membrane collagen (type IV, etc.), FACIT collagen (type IX, type XII, type XIV, type XVI, type XIX, etc.), multiplexins collagen (type XV, type XVIII, etc.), microfibril collagen (type VI, etc.), long-chain collagen (type VII, etc.), membrane-bound collagen (type XIII, type XVII, etc.), and the like, all of which may be used in the present invention. As used herein, the term “basement membrane collagen” refers to collagen which is a major component of basement membrane.

As used herein, “type I collagen” refers to collagen having a structure of [α1(I)₂α2(I)] which consists of two α1(I) chains and three α2 (I) chains, i.e., three polypeptide hetero chains, which form tissue skeleton present in all tissues in organisms, or a molecule having an equivalent function. Examples of the amino acid sequence of such a polypeptide include, but are not limited to, p02454 and p02464 (Genbank Accession Numbers). A molecule having a function equivalent to that of type I collagen can be identified herein by, for example, an enzyme antibody method, an EIA method, or the like.

As used herein, the term “type IV collagen” refers to basement membrane collagen. This collagen consists of four domains 7S, NC2, TH2 and NC1. Four collagen molecules are polymerized at 7S of each N terminus and two molecules are polymerized at NC1 of each C terminus, leading to the formation of a network. The term “type IV collagen” also includes a molecule having an equivalent function. Representatively, examples of the amino acid sequence of the polypeptide include, but are not limited to Genbank Accession Numbers p02462, p08572, U02520, D17391, P29400, U04845, and the like. A molecule having a function equivalent to that of type IV collagen can be herein identified by, for exaample, an enzyme antibody method, an EIA method, or the like.

As used herein, the term “fibronectin” has the same meaning as used in the art and is conventionally categorized into an adhesion factor protein.

As used herein, the term “laminin” has the same meaning as used in the art and is conventionally categorized into an adhesion factor protein. A cell adhesion function thereof has attracted attention and has been rigorously researched. Laminin is a macromolecule glycoprotein constituting basement membrane and its physiological activity is involved in a number of cell functions, such as cell adhesion, elongation, intercellular signal transmission, the growth of normal cells and cancer cells, the induction of cellular differentiation, the metastasis of cancer cells, or the like. Laminin can be purified from Engelbreth-Holm-Swarm mouse tumor or the like. Laminin consists of a α chain, a β chain and a γ chain. 20 or more combinations of chains are known. All laminins can be herein used as a biological molecule capable of binding to a support. All laminins are known to be involved in cell adhesion.

Laminin, collagen, fibronectin, and the like may be available from BD (Becton and Dickinson and Company).

As used herein, the term “crosslinking molecule” refers to a molecule capable of having a covalent bond between a biocompatible material and a biological molecule, between a protein and a protein, between a protein and a nucleic acid, between two strands of DNA, or the like. Examples of such a crosslinking form include, but are not limited to, premature crosslinking (Schiff base crosslinking), mature crosslinking (pyridinoline), aging-associated collagen crosslinking (histidinoalanine), and the like. Such crosslinking may be preferable when a rigid structure, such as a tooth or the like, is desired.

As used herein, the term “support” refers to a material (preferably, a solid) for an implant or a biocompatible implant of the present invention. A support may be in the shape of a patch, a valve, a tube, a membrane, or the like. A material for such a support includes any solid material which is capable of, or is induced to, bind to a biological molecule for use in the present invention via covalent bonding or noncovalent bonding. Therefore, examples of such a support material include, but are not limited to, any material capable of forming a solid surface, e.g., glass, silica, silicone, ceramic, silicon dioxide, plastic, metal (including alloy), naturally-occurring and synthetic polymers (e.g., a biodegradable polymer (e.g., PGA, PLGA, PLA, PCLA), polystyrene, cellulose, chitosan, dextran and nylon), protein, and the like. A support may be formed of a plurality of different materials. When such a material is used in an implant of the present invention, the material is preferably biocompatible. It can be determined whether or not a material is biocompatible, by observing a rejection reaction in biochemical quantification (an SDS-PAG method, a labeled collagen method, etc.), immunological quantification (an enzyme antibody method, a fluorescent antibody method, an immunohistological study, etc.), or the like. More preferably, a support for use in the present invention may be advantageously biodegradable. A component contained in an implant of the present invention becomes unnecessary after a certain period of time, and preferably, degrades and vanishes after that period of time. Examples of such a biodegradable material include, but are not limited to, a biodegradable polymer (e.g., PGA, PLGA, PCLA, etc.). Alternatively, a support for use in the present invention is a component capable of forming a part of an organism. Examples of such a component include, but are not limited to, silicone, ceramic, protein, lipid, nucleic acid, sugar (carbohydrate), and a complex thereof.

As used herein, a “first layer” is intended to face an internal side when a support of the present invention is used as a graft, since the first layer has a rough surface.

As used herein, a “second layer” is intended to face an exterior side when a support of the present invention is used as a graft, since the second layer can withstand in vivo shock.

As used herein, an “intermediate layer” is intended to be sandwiched between a second layer and a first layer of a support. An intermediate layer does not have to be closely attached to a second layer or a first layer, however, the intermediate layer usually needs to be adhered via at least one point to the first layer and the second layer. When sealing is intended, the intermediate layer is preferably closely attached to the second layer or the first layer, and more preferably is closely attached to both of the layers.

Herein, a support of the present invention comprises a first layer and a second layer, in which the two layers are attached via at least one point, and preferably, further comprises an intermediate layer, in which the intermediate layer achieves the attachment. It will be understood that the support may further comprise additional layers (a third layer, a fourth layer, or the like), if required.

As used herein, the term “rough surface” indicates that hole(s) are present on a surface. Preferably, such a surface has a hole having a sufficient space for accommodating a cell. Such a hole capable of accommodating a cell typically has a diameter of at least about 1 μm, preferably at least about 10 μm, more preferably at least about 50 μm, and even more preferably at least 100 μm. With such a rough surface, the first layer of a support of the present invention can function as a scaffold for cells. An example of a layer having a rough surface includes, but is not limited to, a knit.

As used herein, the term “strength which allows to resist in vivo shock” in relation to a material indicates that a material can resist normal in vivo shock at an implanted site. The strength varies depending on the implanted site. However, when the implanted site has been decided, the strength can be immediately understood and determined. Such a strength can be represented by tension strength (representative units: N (force), MPa (stress)), the modulus of elasticity (Young's modulus: representative units: N (force), MPa (stress)), strain (representative unit: %), or the like. An example of a layer having such a strength includes, but is not limited to, a woven.

Herein, the tension strength, the modulus of elasticity, the strain or the like can be determined by a tension test. An illustrative tension test as used herein will be described below.

The tension strength of an implant can be herein determined by a tension tester (TENSILLON ORIENTEC). Specifically, a weight was loaded on a strip material having a width of 5 mm and a length of 30 mm in a minor axis direction at a rate of 10 mm/min so as to measure the strain at break and the modulus of elasticity thereof. Representatively, an implantable implant may have a strength of at least about 10 N, usually at least about 25 N, preferably at least about 50 N, and more preferably at least about 75 N. When used in a conventional organ implantation, the implant may have a strength of at least about 50 N. This is because the implant is not broken. In the above-described protocol, strain is measured as follows. The length of an implant in each direction is measured before and after application of a tensile stimulus. The length after tension is divided by the length before tension and is multiplied by 100 to obtain a strain. When represented by stress, the support of the present invention typically has a tensile strength of at least 1 MPa, preferably at least 5 MPa, and more preferably at least 10 MPa. The support of the present invention typically has a modulus of elasticity of 1 MPa, preferably at least 10 MPa, and more preferably at least 20 MPa. The support of the present invention typically has a strain of at least 105%, and preferably at least 110%. The strain is measured both in the longitudinal direction and in the transverse direction. There is preferably no variation in strain in both of the directions, though the present invention is not limited to this. The strength, the modulus of elasticity and the like may be represented either by N (force) or by MPa (stress). In this case, these representations can be converted to each other in accordance with an expression 1 N/measured mm²=1 MPa.

As used herein, the term “seal” with respect to a support of the present invention indicates that the first and second layers of the support are attached so that a biological molecule cannot substantially communicate between the front side and the rear side of the support. The degree of sealing can be represented by a water leakage rate. A layer capable of being sealed includes, but is not limited to, a synthetic biodegradable polymer.

A water leakage rate can be herein determined by holding a support horizontally, adding 10 ml of water thereon in a dropwise manner, and measuring the amount of leaking water for 60 sec. The water leakage rate is represented by the amount of leaked water itself or divided by 10 ml.

An attachment strength between a certain layer and another layer can be herein determined by a tension test. Specifically, the attachment strength can be measured by the above-described test.

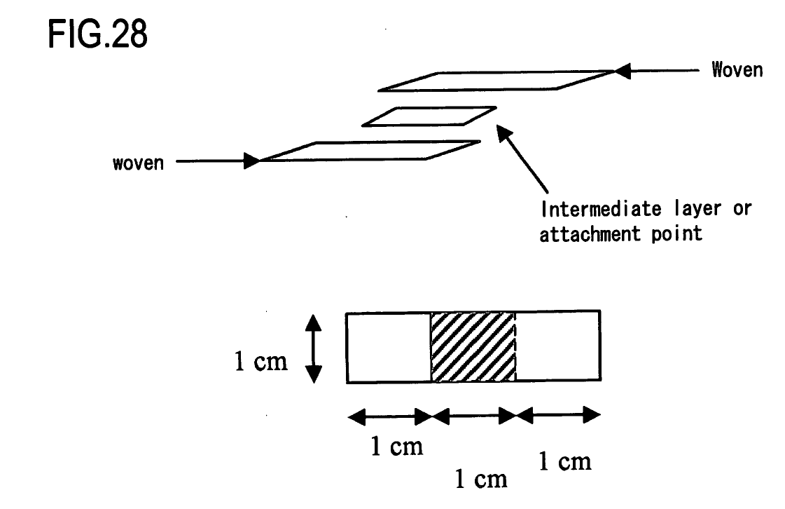

An attachment strength is determined by a tension test as follows. Specifically, a 20 mm-long first layer and a 20 mm-long second layer are attached together overlapping over a length of 10 mm, preferably via an attachment layer (intermediate layer) to produce a 30 mm-long strip support. A load is put on the support in a longitudinal direction thereof at a rate of 10 mm/min. A load at break is defined as attachment strength. The measurement is schematically shown in FIG. 28. Measurement of attachment strength is described in, for example, Otani et al., Biomaterials, 17 (1996) 1387-1391.

As used herein, the term “knit” refers to a fabric produced by intertwining (combining) loops of a material (typically, in the form of thread) using a needle or wire. A knit is used when a space is required in a fabric. Since loops are connected in a knit, there is a gap between each loop which provides a sufficient space for accommodating a cell. However, when only a knit is used, liquid (e.g., blood) disadvantageously leaks through gaps.

As used herein, the term “woven” refers to a fabric produced by interlacing a material (typically, in the form of thread), representatively interlacing the threads of the weft and the warp. There is substantially no gap in a woven, whereby a woven is used when prevention of leakage of liquid (e.g., blood) is desired. However, when only a woven is used, there is a problem that the woven is frayed when it is stitched.

As used herein, the term “sufficient space for accommodating a cell(s)” in relation to a support or layer refers to a sufficient space with which a cell can be at least attached to the support or layer, preferably in which a cell can be accommodated. Such a space has a diameter of at least 10 μm, preferably at least 50 μm, and more preferably at least 100 μm. A sufficient space for accommodating a cell may have a diameter smaller than the above-described lowest value as long as the cell can be attached to the support or layer. Preferably, such a space has a size such that liquid is not likely to leak. Therefore, the diameter of the space may be, for example, 200 μm, though the present invention is not limited to this.

As used herein, the term “biocompatible” refers to a property of being compatible with a biological tissue or organ without eliciting toxicity, an immune reaction, an injury or the like. In the present invention, the term “biocompatible” in relation to a certain material indicates that when the material is used as it is, the material is biocompatible. Further, when a means for preventing the above-described toxicity, immune reaction or injury can be provided if required, the material can be said to be biocompatible, although the material is toxic if it is used singly. Such a means (e.g., administration of an immunosuppressant, etc.) can be used to significantly reduce or substantially extinguish toxicity, an immune reaction or an injury. If a material is not biocompatible when it is used singly, such a prevention means is preferably contained in an implant of the present invention. Examples of a biocompatible material which may be used in the present invention, include, but are not limited to, PGA, PLA, PCLA, PLGA, poly(L-lactic acid), polybutylate, silicone, biodegradable calcium phosphate, porous 4-fluorinated ethylene resin, polyproplylene, amylose, cellulose, synthetic DNA, polyesters, and the like.

As used herein, the term “biodegradable material” refers to any material which can be naturally degraded, metabolized in an organism, or degraded by a microorganism. As a biodegradable material, a biodegradable polymer is typically used.

As used herein, the terms “biodegradable polymer” and “biodegradable macromolecule” are used interchangeably, referring to a macromolecule which can be naturally degraded, metabolized in an organism, or degraded by action of a microorganism. Such a biodegradable polymer is degraded by hydrolysis into water, carbon dioxide, methane, or the like. Such a biodegradable polymer is either a naturally-occurring or synthetic macromolecule. Examples of a naturally-occurring macromolecule include, but are not limited to, a protein (e.g, collagen, etc.) and a polysaccharide (e.g, starch, etc.). Examples of a synthetic macromolecule include, but are not limited to, aliphatic polyesters, such as poly(glycolic acid), poly(L-lactic acid), polyethylene succinate, and the like. Such a biodegradable polymer has been used in applications, such as a surgical absorbable suture, a base for sustained release drug, a bone joining material, and the like. Any polymers which are used in such applications can be employed in the present invention. Examples of a biodegradable polymer include, but are not limited to, polypeptide, polysaccharide, nucleic acid, PGA, PLGA, poly(L-lactic acid), polybutylate, maleic acid copolymer, lactid-caprolactone copolymer, poly-ε-caprolactone, poly-β-hydroxycarboxylic acid, polydioxanone, poly-1,4-dioxepane-7-one, glycolide-trimethylenecarbonate copolymer, poly(sebacic acid) anhydride, poly-ω-(carboxyphenoxy)alkylcarbonic anhydride, poly-1,3-dioxane-2-one, polydepsipeptide, poly-α-cyanoethylacrylate, polyphosphagen, hydroxyapatite, and the like. Such a biodegradable polymer may be preferably stable in organisms for a predetermined time, and subsequently degraded or absorbed. Such degradation is performed by action of an enzyme for metabolism (specific degradation mechanism) or by contact with body fluid (non-specific degradation mechanism). A material which can be degraded by either or both of the mechanisms can be used in the present invention. Preferably, such a biodegradable polymer is non-toxic and/or non-antigenic. Also, the intermediate products and final products of degradation or metabolism are preferably non-toxic and/or non-antigenic.

As used herein, the term “PGA” is an abbreviation of poly(glycolic acid), which is a polymer of glycolic acid. Glycolic acid is represented by CH₂(OH)COOH. PGA is also called polyglycolide. Poly(glycolic acid) is suitable for production of a knit. Therefore, in the present invention, representatively, PGA may be used for a first layer having a rough surface, though the present invention is not limited to this.

As used herein, the term “PLA” is an abbreviation of poly(L-lactic acid), which is a polymer of L-lactic acid. Glycolic acid is represented by CH₃CH(OH)COOH. PLA is also called polylactid. Poly(L-lactic acid) is suitable for production of a woven. In the present invention, therefore, PLA is used for a second layer having a strength which allows the second layer to resist in vivo shock, though the present invention is not limited to this.

PGA and PLA can be synthesized by a method well known in the art. Examples of such a method include, but are not limited to, thermal dehydrocondensation of glycolic acid or lactic acid, polymerization of dehydrohalogenated α-haloacetic acid, α-halopropionic acid, and the like. Preferably, in order to increase the degree of polymerization, an obtained oligomer is subjected to heat degradation under reduced pressure to obtain a cyclic dimer of glycolide or lactid. The dimers are subjected to ring opening polymerization so that a macromolecule having a desired degree of polymerization can be synthesized (e.g., H. R. Kricheldorf, et al., Makromol. Chem. Suppl. 12, 25(1985)). In this case, it is preferable that a catalyst remaining after polymerization is not biologically toxic. Examples of such a catalyst include, but are not limited to, Tin(II) 2-ethylhexanoate and the like. Any catalyst in the art which has no or low toxicity can be used.

As used herein, the term “PLGA” is an abbreviation of a poly(L-lactic acid)-poly(glycolic acid) copolymer which is a copolymer made of glycolic acid and lactic acid. Lactic acid is represented by CH₃CH(OH)COOH. PLGA is called polyglactin (e.g., glycolide/lactid=9/1).

PLGA can be synthesized by a method well known in the art. The properties of PLGA can be dramatically altered by changing the ratio of glycolic acid and lactic acid contained therein. For example, the absorption half life of PLGA in organisms can be changed within the range of from several days to several months in accordance with a relational expression as described in, for example, R. A. Miller et al., J. Biomed. Res., 11, 719(1977). When the desired in vivo half life is within 2 to 3 weeks, the ratio of PLA to PGA is typically 20:80 to 80:20. When the desired in vivo half life is one month or more, the ratio of PLA to PGA is preferably 20:80 to 0:100 or 80:20 to 100:0. Therefore, when a longer absorption half life (e.g., several months) is desired, PLA or PGA is preferably used. The half life of the fiber strength of PLGA can be altered by changing the ratio of PLA to PGA. The half life of fiber strength is 2 to 3 weeks for PGA plus PLA and 3 to 6 months for PLA. Therefore, when a longer fiber strength half life is desired, PLGA containing PLA in an increased proportion or PLA itself is preferably used.

PLGA can be synthesized by a method well known in the art. For example, glycolide and lactid produced by the above-described synthesis of PLA and PGA are used as a mixture and the mixture is subjected to ring opening coplymerization. PLGA which has a glycolide-to-lactid ratio of from 25:75 to 75:20 is typically a glass-like macromolecule. PLGA having a glycolide-to-lactid ratio of from 25:75 to 0:100 is a crystalline macromolecule similar to poly(L-lactic acid) PLGA having a glycolide-to-lactid ratio of from 75:25 to 100:0 is a crystalline macromolecule similar to poly(glycolic acid). Therefore, the hydrolysis ability or material strength of PLGA can be altered by changing the compostion thereof by those skilled in the art.

As used herein, the term “mesh-like” in relation to the form of an implant or the like refers to a network form. A mesh-like implant can be produced by a method well known in the art. The fine structure of such a mesh-like implant can be created by a method well known in the art. As such a mesh-like implant, for example, a commercially available product (VICRYL KNITTED MESH (manufactured by ETHICON)) can be used.

As used herein, the term “sponge-like” in relation to the form of an implant or the like refers to porous. Such a sponge-like implant can be produced by a method well known in the art. As such a sponge-like implant, for example, a commercially available product (VICRYL WOVEN MESH (manufactured by ETHICON)) can be used.

As used herein, the term “coating” in relation to a support or the like refers to a state that the support is covered with another material. Therefore, coating can be performed using a material capable of interacting with the support. A support may be coated so that a material constituting the support is not exposed to the outside (e.g., air) of the support. Coating may not be performed to such an extent that a material constituting the support is not exposed to the outside, if the support and a coating material can interact with each other. The degree of coating can be arbitrarily adjusted by those skilled in the art using a method well known in the art. Such a coating technique is described in, for example, “Kobunshikino Zairyo Shirizu Iryokino Zairyo [Molecular Function Series: Medical Functional Material], Kyoritsu Shuppan K. K.

As used herein, the terms “polysaccharide”, “oligosaccharide”, “sugar”, “saccharide” and “carbohydrate” are used interchangeably, referring to a macromolecule compound obtained by dehydrocondensation of monosaccharides via glycoside bond. The term “monosaccharide” refers to a carbohydrate which cannot be decomposed by hydrolysis to a simpler molecule and which is represented by general formula CₙH₂ₙOₙ (n=2, 3, 4, 5, 6, 7, 8, 9 and 10; corresponding to diose, triose, tetrose, pentose, hexose, heptose, octose, nonose and decose, respectively). Generally, a monosaccharide containing an aldehyde or ketone group corresponding to a chained polyvalent alcohol is called aldose or ketose, respectively. Such a polysaccharide may be used singly or in combination in a support of the present invention.

As used herein, the term “lipid” refers to a biological material which is difficult to dissolve in water and is soluble in an organic solvent. Lipid includes a number of types of organic compounds. Typically, lipid includes long-chain fatty acid and a derivative or analog thereof and herein includes organic compounds present in an organism, which are insoluble in water and soluble in an organic solvent, such as steroids, carotenoids, terpenoids, isoprenoids, fat-soluble vitamins, and the like. Examples of lipid include, but are not limited to, 1) simple lipids (esters of fatty acids and various alcohols; also called neutral lipid), such as fats and oils (triacylglycerol), waxes (fatty esters of higher alcohols), sterol ester, a fatty ester of a vitamin, etc.; 2) composite lipids (compounds having a polar group, such as phosphoric acid, sugars, sulfuric acid, amines, or the like, in addition to fatty acids and alcohols), such as glycerophospholipids, sphingophospholipids, glyceroglycolipids, sphingoglycolipids, lipids having C—P bond, sulpholipid, and the like; 3) induced lipids (compounds obtained by hydrolysis of simple lipids and composite lipids, which are fat-soluble), such as fatty acids, higher alcohols, fat-soluble vitamins, steroids, hydrocarbon, and the like. In the present invention, any lipid can be used as a support as long as it does not inhibit the function of the support of aggregating cells.

As used herein, the term “composite” or “complex” in relation to a material refers to a molecule comprising a plurality of types of substances (preferably, these components interact with one another). Examples of such a complex include, but are not limited to, glycoproteins, glycolipids, and the like.

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intended nucleic acid; and in the case of proteins, agents other than proteins and proteins having an amino acid sequence other than an intended protein). The “isolated” nucleic acids and proteins include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to one from which at least a part of naturally accompanying agents is removed. Therefore, ordinarily, the purity of a purified biological agent is higher than that of the biological agent in a normal state (i.e., concentrated).

The biological molecule for use in the present invention may be collected from an organism or may be synthesized using a method known to those skilled in the art. For example, synthesis techniques using automatic solid-phase peptide synthesizers are described in, for example, Stewart, J. M. et al. (1984), Solid Phase Peptide Synthesis, Pierce Chemical Co.; Grant, G. A. (1992), Synthetic Peptides: A User's Guide, W.H. Freeman; Bodanszky, M. (1993), Principles of Peptide Synthesis, Springer-Verlag; Bodanszky, M. et al. (1994), The Practice of Peptide Synthesis, Springer-Verlag; Fields, G. B. (1997), Phase Peptide Synthesis, Academic Press; Pennington, M. W. et al. (1994), Peptide Synthesis Protocols, Humana Press; and Fields, G. B. (1997), Solid-Phase Peptide Synthesis, Academic Press. Such other molecules can be synthesized using a method well known in the art.

As used herein, “homology” of a biological molecule (e.g., a nucleic acid sequence, an amino acid sequence, or the like, encoding collagen, laminin, and the like) refers to the proportion of identity between two or more sequences. As used herein, the identity of a sequence refers to the proportion of the identical sequence between two or more comparable sequences. Therefore, the greater the homology between two given sequences, the greater the identity or similarity between their sequences. Whether or not two sequences have homology is determined by comparing their sequences directly or by a hybridization method under stringent conditions. When two sequences are directly compared with each other, these sequences have homology if the sequences of the genes have representatively at least 50% identity, preferably at least 70% identity, more preferably at least 80%, 90%, 95%, 96%, 97%, 98%, or 99% identity with each other. As used herein, “similarity” of a biological molecule (e.g., a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value. In the present invention, such sequences having a high identity or similarity may be useful.

The similarity, identity and homology of amino acid sequences and base sequences are herein compared using PSI-BLAST (sequence analyzing tool) with the default parameters. Otherwise, FASTA (using default parameters) may be used instead of PSI-BLAST.

As used herein, the term “amino acid” may refer to a naturally-occurring or nonnaturally-occurring amino acid. The term “amino acid derivative” or “amino acid analog” refers to an amino acid which is different from a naturally-occurring amino acid and has a function similar to that of the original amino acid. Such amino acid derivatives and amino acid analogs are well known in the art.

The term “naturally-occurring amino acid” refers to an L-isomer of a naturally-occurring amino acid. The naturally-occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise mentioned, all amino acid described herein are L-isomers. Embodiments using D-amino acids also fall within the scope of the present invention.

The term “nonnaturally-occurring amino acid” refers to an amino acid which is ordinarily not found in nature. Examples of nonnaturally-occurring amino acids include norleucine, para-nitrophenylalanine, homophenylalanine, para-fluorophenylalanine, 3-amino-2-benzyl propionic acid, D- or L-homoarginine, and D-phenylalanine. The term “amino acid analog” refers to a molecule having a physical property and/or function similar to that of amino acids, but is not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid refers to an amino acid or nucleotide in a given protein or polypeptide molecule, which has, or is anticipated to have, a function similar to that of a predetermined amino acid in a protein or polypeptide as a reference for comparison. Particularly, in the case of enzyme molecules, the term refers to an amino acid which is present at a similar position in an active site and similarly contributes to catalytic activity. For example, in the case of antisense molecules, the term refers to a similar portion in an ortholog corresponding to a particular portion of the antisense molecule.

As used herein, the term “corresponding” gene (e.g., a polypeptide or polynucleotide molecule) refers to a gene (e.g., a polypeptide or polynucleotide molecule) in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. For example, a gene encoding mouse collagen corresponds to a gene encoding human collagen.

As used herein, the term “fragment” refers to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. In the present invention, it will be understood that when a polypeptide or polynucleotide is used as a biological molecule, such a fragment may be used as in the full-length molecule as long as a desired object (e.g., a cell attracting effect, etc.) can be achieved.

As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification.

As used herein, the term “biological activity” refers to activity possessed by an agent (e.g., a polypeptide, a protein, etc.) within an organism, including activities exhibiting various functions. For example, when a give agent is an antisense molecule, the biological activity thereof includes binding to a target nucleic acid, suppression of expression by the binding, or the like. For example, when a given agent is an enzyme, the biological activity thereof includes the emzymatic activity thereof. In another example, when a given agent is a ligand, the biological activity thereof includes binding of the agent to a receptor for the ligand. Such biological activity can be measured with a technique well known in the art.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081(1991); Ohtsuka et al., J. Biol. Chem. 260:2605-2608 (1985): Rossolini et al., Mol. Cell. Probes 8:91-98(1994)).

A given amino acid may be substituted with another amino acid in a protein structure, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA code sequence level, to produce a protein which maintains the original property after the substitution. Therefore, various modifications of peptides as disclosed herein and DNA encoding such peptides may be performed without clear losses of biological usefulness.

When the above-described modifications are designed, the hydrophobicity indices of amino acids may be taken into consideration. The hydrophobic amino acid indices play an important role in providing a protein with an interactive biological function, which is generally recognized in the art (Kyte, J. and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2): glutamic acid (−3.5); glutamine (−3.5); aspartic acid (−3.5): asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5. It is understood in the art that such an amino acid substitution based on hydrophobicity is efficient. As described in U.S. Pat. No. 4,554,101, amino acid residues are given the following hydrophilicity indices: arginine (+3.0); lysine (+3.0); aspartic acid (+3.01); glutamic acid (+3.0±1): serine (+0.3); asparagine (+0.2); glutamine (+0.2); glycine (0); threonine (−0.4); proline (−0.5±1); alanine (−0.5); histidine(−0.5): cysteine(−1.0); methionine(−1.3); valine(−1.5); leucine(−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably +0.5.

The term “conservative substitution” as used herein refers to amino acid substitution in which a substituted amino acid and a substituting amino acid have similar hydrophilicity indices and/or hydrophobicity indices. For example, conservative substitution is carried out between amino acids having a hydrophilicity or hydrophobicity index of within ±2, preferably within ±1, and more preferably within ±0.5. Examples of conservative substitution include, but are not limited to, substitutions within each of the following residue pairs: arginine and lysine; glutamic acid and aspartic acid; serine and threonine; glutamine and asparagine; and valine, leucine, and isoleucine, which are well known to those skilled in the art. Such a variant can be used as a biological molecule of the present invention as long as it can achieve the desired object.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Such a variant can be used as a biological molecule of the present invention as long as it can achieve the desired object. The term “allele” as used herein refers to a genetic variant located at a locus identical to a corresponding gene, where the two genes are distinguished from each other. Therefore, the term “allelic variant” as used herein refers to a variant which has an allelic relationship with a given gene. Such an alleic gene mutant has the same or similar sequence to a corresponding alleic gene and typically has substantially the same biological activity, and may rarely have different biological activity. The term “species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining such a species homolog is clearly understood from the description of the present specification. The term “ortholog” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide.

As used herein, the term “substitution, addition or deletion” for a polypeptide or a polynucleotide refers to the substitution, addition or deletion of an amino acid or its substitute, or a nucleotide or its substitute, with respect to the original polypeptide or polynucleotide, respectively. This is achieved by techniques well known in the art, including a site-specific mutagenesis technique and the like. A polypeptide or a polynucleotide may have any number (>0) of substitutions, additions, or deletions. The number can be as large as a variant having such a number of substitutions, additions or deletions which maintains an intended function (e.g., the information transfer function of hormones and cytokines, etc.). For example, such a number may be one or several, and preferably within 20% or 10% of the full length, or no more than 100, no more than 50, no more than 25, or the like.

The term “cell” is herein used in its broadest sense in the art, referring to a structural unit of tissue of a multicellular organism, which is capable of self replicating, has genetic information and a mechanism for expressing it, and is surrounded by a membrane structure which isolates the living body from the outside. In the method of the present invention, any cell can be used as a subject. The number of cells used in the present invention can be counted through an optical microscope. When counting using an optical microscope, the number of nuclei is counted. Tissues are sliced into tissue sections, which are then stained with hematoxylin-eosin (HE) to variegate nuclei derived from extracellular matrices and cells. These tissue sections are observed under an optical microscope and the number of nuclei in a particular area (e.g., 200 μm×200 μm) can be estimated to be the number of cells.

Cells may elicit calcification and immune reactions. Therefore, non-self cells should be removed as much as possible for implantation of tissue or organs. In the case of self cells, decellularization is not required, since no immunological rejection problem is usually raised. However, since decellularization is sometimes preferable, cells should also be removed as much as possible. There is a strong desire for decellularized tissue.

Cells used in the present invention may be derived from any organism (e.g., vertebrates and invertebrates). Preferably, cells derived from vertebrates are used. More preferably, cells derived from mammals (e.g., primates, rodents, etc.) are used. Even more preferably, cells derived from primates are used. Most preferably, cells derived from a human (self, or an individual genetically similar to or the same as self) are used when the cells are implanted into the human.

As used herein, the term “cell replacement” indicates that cells originally existing are replaced with other infiltrating cells in tissue. This term is also referred to as “cellular infiltration”. When an implant of the present invention is used, cells are replaced with cells within a host in implantation. When an implant of the present invention was used, it was demonstrated that host-derived cells infiltrated and replaced after implantation although no self-derived cells were present. Such an event had never occurred in conventional grafts. This finding itself shows an unexpected, extremely excellent effect of the present invention. Cell replacement can be confirmed by a method known in the art. For example, a marker capable of confirming the growth of self cells, such as von Willebrand factor, α-SMA, van Gieson for elastic tissue, or the like, can be used to determine cell replacement. Such a technique for confirming cell replacement is described in, for example, “Byori Soshiki Senshoku Handobukku [Pathologic Tissue Staining Handbook”, Igakushoin.

As used herein, the term “tissue” refers to a group of cells having the same function and form in cellular organisms. In multicellular organisms, constituent cells are usually differentiated so that the cells have specialized functions, resulting in division of labor. Therefore, multicellular organisms are not simple cell aggregations, but constitute organic or social cell groups having a certain function and structure. Examples of tissues include, but are not limited to, integument tissue, connective tissue, muscular tissue, nervous tissue, and the like. Tissue targeted by the present invention may be derived from any organ or part of an organism. In a preferred embodiment of the present invention, tissue targeted by the present invention includes, but is not limited to, blood vessels, blood vessel-like tissue, cardiac valves, pericardia, dura mater, heart, cardioendothelium, skin, bone, soft tissue, trachea, and the like. A molecule for use in a support of the present invention is preferably biocompatible. Therefore, the present invention can be in principle applied to implantation of tissue derived from any organ. Therefore, tissue targeted by the present invention may be derived from any organ or part of an organism or may be derived from any species of organism. An organism targeted by the present invention includes a vertebrate or an invertebrate. Preferably, an organism targeted by the present invention includes mammals (e.g., primates, rodents, etc.). Even more preferably, an organism targeted by the present invention includes primates. Most preferably, an organism targeted by the present invention includes a human.

As used herein, the term “implant” or “explant” refers to a part or whole of a tissue or organ or a material capable of becoming a part or whole of a tissue or organ. An implant can be artificially synthesized or may be a naturally-occurring material, or may be a combination thereof. An implant may comprise a support for retaining the shape thereof. A support of the present invention can be here used singly or in combination with a biological molecule, as an implant. Preferably, an artificial material is used as an implant in the present invention.

As used herein, the term “implant” and the terms “graft” and “tissue graft” are used interchangeably, referring to homologous or heterologous tissue or cell group or an artificial synthetic material which is inserted into a particular site of a body and thereafter forms a portion of the body. Examples of conventional grafts include, but are not limited to, organs or portions of organs, blood vessels, blood vessel-like tissue, skin segments, cardiac valves, pericardia, dura mater, corneas, bone segments, teeth, and the like. Therefore, grafts encompass any one of these which is inserted into a deficient portion so as to compensate for the deficiency. Grafts include, but are not limited to, autografts, allografts, and xenografts, which depend on the type of their donor.

As used herein, “membrane-like tissue” is also referred to as “planar tissue” and refers to a tissue having a membrane form. Membrane-like tissue includes tissue from organs, such as pericardia, dura mater, and corneas.

As used herein, the term “tube-like tissue” refers to a tissue in the form of a tissue. A tube-like tissue includes a tissue of an organ, such as a blood vessel or the like.

As used herein, the term “organ” or “part” is used interchangeably, referring to a structure which is a specific portion of an individual organism where a certain function of the individual organism is locally performed and which is morphologically independent. Generally, in multicellular organisms (e.g., animals and plants), organs are made of several tissues in specific spatial arrangement and tissue is made of a number of cells. Examples of organs or parts include organs or parts related to a blood vessel system. In one embodiment, examples of organs targeted by the present invention include ischemic organs (the heart undergoing cardiac infarction, skeletal muscle undergoing ischemia, and the like). In one preferred embodiment, organs targeted by the present invention are heart, liver, kidney, stomach, intestine, brain, bone, trachea, skin, blood vessel, soft tissue. In a more preferred embodiment, organs targeted by the present invention are heart (cardiac valve), bone, skin, blood vessel, and the like.

As used herein, the term “immune reaction” refers to a reaction due to loss of coordination of immunologic tolerance between a graft and a host, including, for example, hyperacute rejection reactions (within several minutes after implantation; immune reactions due to an antibody, such as β-Gal or the like), acute rejection reactions (cell-mediated immune reactions about 7 to 21 days after implantation), chronic rejection reactions (rejection reactions due to cell-mediated immune response after three months), and the like.

As used herein, elicitation of immune reactions can be determined by histological examination of the type, number, or the like, of cells (immune cells) infiltrating implanted tissue by observing under a microscope tissue sections stained by HE staining or immunological staining.

As used herein, the term “calcification” refers to precipitation of calcareous substances in organisms. When a tissue or organ undergoes calcification in vivo, the normal functions of the tissue or organ is usually impaired. It is preferably that calcification does not occur. Therefore, implantation therapy conventionally requires treatment for avoiding calcification. An implant of the present invention can avoid the calcification problem.

As used herein, “calcification” in vivo can be determined by measuring calcium concentration. Specifically, implanted tissue is taken out; the tissue section is dissolved by acid treatment or the like; and the atomic absorption of the solution is measured by a trace element quantifying device.

As used herein, the term “within organism(s) (or in organism(s))” or “in vivo” refers to the inner part of organism(s). In a specific context, “within organism(s)” refers to a position at which a subject tissue or organ is placed.

As used herein, “in vitro” indicates that a portion of an organism is extracted or released outside the organism for various purposes of research (e.g., in a test tube). The term in vitro is in contrast to the term in vivo.

As used herein, the term “ex vivo” refers to a series of operations where target cells into which a gene will be introduced are extracted from a subject; a therapeutic gene is introduced in vitro into the cells; and the cells are returned into the same subject.

As used herein, the term “autograft” refers to a graft which is implanted into the same individual from which the graft is derived. As used herein, the term “autograft” may encompass a graft from a genetically identical individual (e.g. an identical twin) in a broad sense.

As used herein, the term “allograft” refers to a graft which is implanted into an individual which is the same species but is genetically different from that from which the graft is derived. Since an allograft is genetically different from an individual (recipient) to which the graft is implanted, the graft may elicit an immune reaction. Such a graft includes, but is not limited to, for example, a graft derived from a parent.

As used herein, the term “xenograft” refers to a graft which is implanted from a different species. Therefore, for example, when a human is a recipient, a porcine-derived graft is called a xenograft.

As used herein, “recipient” (acceptor) refers to an individual which receives a graft or implanted matter and is also called “host”. In contrast, an individual providing a graft or implanted matter is called “donor” (provider).

As used herein, the term “subject” refers to an organism to which treatment of the present invention is applied and is also referred to as “patient”. A patient or subject may be preferably a human.

As used herein, the term “pharmaceutically acceptable carrier” refers to a material for use in production of a medicament or animal drug, which does not have an adverse effect on an effective component. Examples of such a pharmaceutically acceptable carrier include, but are not limited to, antioxidants, preservatives, colorants, flavoring agents, diluents, emulsifiers, suspending agents, solvents, fillers, bulking agents, buffers, delivery vehicles, agricultural or pharmaceutical adjuvants, and the like.

### DESCRIPTION OF PREFERRED EMBODIMENTS

Hereinafter, the present invention will be described by way of best mode embodiments. Embodiments described below are provided only for illustrative purposes. Accordingly, the scope of the present invention is not limited except as by the appended claims. It will be clearly understood by those skilled in the art that modifications and variations of the present invention can be achieved by considering the description of the present specification.

(Biological Molecule-Attached Explant)

According to an aspect of the present invention, a biocompatible implant is provided. The biocompatible implant comprises A) a biological molecule: and B) a support. It was unexpectedly found that the biocompatible implant comprising only the biological molecule and the support can be used for actual implantation therapy and can be cellularized after implantation. Conventionally, it was considered that usable grafts are limited to self-reproducing biological materials (e.g., a part of a tissue, an organ itself, etc.) and artificial materials with a self-reproducing biological material (e.g., a cell, etc.).

As will be seen from examples of the present invention, the present inventors found that even when an implant containing no self-reproducing material (e.g., a cell, etc.) is implanted, the implant is in situ cellularized (i.e., self cells or equivalents thereof aggregate and multiply). Therefore, the implant of the present invention can be used for treatment of a tissue or an organ which is conventionally considered not to be possible. This is because a support contained in the implant of the present invention can be adapted to be in any shape.

Though not wishing to be bound by any theory, when the implant of the present invention is implanted into a part of an organ or tissue within a host (representatively, an injured site or a site which is desired to be reinforced), the biological molecule (e.g., collagen, etc.) contained in the implant causes cells within the host, particularly which will constitute a part of the organ or tissue (e.g., growth or differentiation), to aggregate in the vicinity of the implant. In some cases, the cell growth repairs or reinforces an injured site or a site which is desired to be reinforced in the organ or tissue.

Therefore, any biological molecule capable of directly or indirectly causing cells within a host to aggregate (e.g., adhesion, induction of molecules mediating adhesion, etc.) can be employed. Therefore, the biological molecule may be derived from an organism, or alternatively may be produced by synthesis as long as it has the above-described function. The biological molecule may be either naturally occurring or non-naturally occurring. Preferably, the biological molecule is advantageously a naturally-occurring material which has been revealed to be not harmful to hosts (e.g., a material which is approved to be used as a component of a medicament by the Health, Labor and Welfare Ministry, such as a product described in the Japanese Pharmacopoeia or the like). Alternatively, the biological molecule may be a material which has been separately confirmed not to be harmful to hosts. Representatively, the biological molecule includes a protein.

In one embodiment, a biological molecule for use in the present invention may include a cellular physiologically active substance. Examples of the cellular physiologically active substances include, but are not limited to, an HGF, a platelet derived growth factor (PDGF), an epidermal growth factor (EGF), a fibroblast growth factor (FGF), a hepatocyte growth factor (HGF), a vascular endothelial growth factor (VEGF), a leukemia inhibiting factor (LIF), a c-kit ligand (SCF), and the like.

In a preferred embodiment, a biological molecule for use in the present invention may include a cell adhesion molecule. The cell adhesion molecule is considered to be preferable since it mediates cell-to-cell or cell-to-substrate adhesion. It is believed that when a cell adhesion molecule is implanted to a host, cells within the host are aggregated in situ. However, it is conventionally unclear as to whether or not the cell adhesion molecule is directly used as a graft. Rather, the cell adhesion molecule has been believed to be essentially used together with a self-reproducing material, such as a cell (see Raf Sodian, et al., Ann. Throrac. Surgery, 2000, 70, 140-44; Sodian R., Lemke T., Fritsche C., Hoerstrup S. P., Fu P., Potapov E. V., Hausmann H., Hetzer R., Tissue Eng., 2002 Oct., 8(5): 863-70; Kadner A., Hoerstrup S. P., Zund G., Eid K., Maurus C., Melnitchouk S., Grunenfelder J., Turina M. I., Eur J., Cardiothorac. Surg., 2002 Jun., 21(6): 1055-60; etc.). Thus, the graft of the present invention provides an unexpected effect.

Examples of the cell adhesion molecule include, but are not limited to, collagen, ICAM, NCAM, fibronectin, collagen, vitronectin, laminin, integrin, vitronectin, fibrinogen, an immunoglobulin superfamily member, and the like.

In another preferred embodiment, a biological molecule for use in the present invention includes an extracellular matrix. The extracellular matrix is also known to have activity to aggregate cells. However, it is conventionally unclear as to whether or not the extracellular matrix is directly used as a graft. Rather, the extracellular matrix has been believed to be used essentially together with a self-reproducing material, such as a cell. Thus, the finding that an extracellular matrix can be directly used as a major component of a graft is an unexpected effect.

Examples of the extracellular matrix include, but are not limited to, collagen, elastin, proteoglycan, glycosaminoglycan, fibronectin, laminin, and the like.

In another preferred embodiment, a biological molecule for use in the present invention includes a cellular adhesive protein. The cellular adhesive protein is also known to have activity to aggregate cells. However, it is conventionally unclear as to whether or not the cellular adhesive protein is directly used as a graft. Rather, the cellular adhesive protein has been believed to be essentially used together with a self-reproducing material, such as a cell. Thus, the finding that the cellular adhesive protein can be directly used as a major component of a graft is an unexpected effect.

Examples of the cellular adhesive protein include, but are not limited to, collagen, laminin, fibronectin, ICAM, NCAM, fibronectin, collagen, vitronectin, laminin, integrin, vitronectin, fibrinogen, an immunoglobulin superfamily member, and the like.

In one preferred embodiment, a biological molecule for use in the present invention includes an RGD molecule. The RGD molecule is also known to have activity to aggregate cells. However, it is conventionally unclear as to whether or not the RGD molecule is directly used as a major component of a graft. Rather, the RGD molecule has been believed to be essentially used together with a self-reproducing material, such as a cell. Thus, the finding that the RGD molecule can be directly used as a major component of a graft is an unexpected effect.

Examples of the RGD molecule include, but are not limited to, collagen (type I, etc.), laminin, fibronectin, ICAM, NCAM, vitronectin, von Willebrand factor, entactin, and the like.

In a more preferred embodiment, a biological molecule for use in the present invention includes collagen or laminin. Collagen and laminin are also known to have activity to aggregate cells. However, collagen and laminin are conventionally used as an auxiliary component. It is conventionally unclear as to whether or not collagen and laminin are directly used as a major component of a graft. Rather, collagen and laminin have been believed to be essentially used together with a self-reproducing material, such as a cell. Thus, the finding that collagen and laminin can be directly used as a major component of a graft is an unexpected effect.

More preferably, the above-described collagen may be fiber forming collagen or basement membrane collagen. More preferably, a biological molecule for use in the present invention includes fiber forming collagen and basement membrane collagen. The inclusion of both fiber forming collagen and basement membrane collagen promoted the implant to be cellularized to the highest degree after implantation. Though not wishing to be bound by any theory, the reason is considered to be that the aggregating of cells and adhesion activity are optimized by the combination.

More preferably, the above-described collagen may be advantageously of type I or type IV. The reason type I and type IV are advantageous is, but is not limited to, that these collagens are effective for scaffolding survival or growth of vascular endothelial cells, smooth muscle cells, cardiac muscle cells, and progenitor cells (stem cells) thereof.

In a most preferred embodiment, a biological molecule of the present invention includes both type I collagen and type IV. The inclusion of both type I collagen and type IV collagen promoted the implant to be cellularized to the highest degree after implantation. Though not wishing to be bound by any theory, the reason is considered to be that the aggregating of cells and adhesion activity are optimized by the combination.

In another embodiment, a support for use in the present invention may be in the shape of membrane. An implant comprising the membrane-like support may be appropriate for implantation into a membrane-like tissue or organ. Examples of the membrane-like tissue or organ include, but are not limited to, skin, cornea, dura mater, a part of a large-size organ (e.g., liver, heart, etc.), and the like.

In another embodiment, a support for use in the present invention may be in the shape of a tube. An implant comprising the tube-like support may be appropriate for implantation into a tube-like tissue or organ. Examples of the tube-like tissue or organ include, but are not limited to, a blood vessel, a lymphatic vessel, and the like.

In another embodiment, a support for use in the present invention may be in the shape of a valve. An implant comprising the valve-like support may be appropriate for implantation into a valve-like tissue or organ. Examples of the valve-like tissue or organ include, but are not limited to, a cardiac valve and the like.

In a preferred embodiment, a support of the present invention may advantageously include a biodegradable polymer. More preferably, a support of the present invention may be advantageously composed of a biodegradable polymer. When the support includes a biodegradable polymer or is composed of a biodegradable polymer, the implant of the present invention will be composed only of self cells after a certain period of time has passed. In this case, an implanted organ or tissue cannot be substantially distinguished from a corresponding self organ or tissue. Examples of a biodegradable polymer for use in the present invention include, but are not limited to, PLA, PGA, PLGA, polycaprolactum (PCLA), and the like.

In a preferred embodiment, a support for use in the present invention includes at least one component selected from the group consisting of PGA and PLGA. More preferably, a support for use in the present invention includes a PLGA having a glycolic acid-to-lactic acid ratio of about 90: about 10 to about 80: about 20. By using PLGA having such a ratio, appropriate levels of strength and half life (about one to several months) can be achieved. The strength may be, for example, at least about 10N, normally about 25N or more, and preferably about 50 N or more. More preferably, the strength is about 75 N or more.

In another preferred embodiment of the present invention, a cell adhesion molecule can be used as a support for use in the present invention. The cell adhesion molecule includes those described above. Preferably, the cell adhesion molecule may advantageously have a strength which allows for the support. The strength is, for example, about 10 N or more, about 20 N or more, and about 25 N or more, preferably about 50 N or more, and more preferably about 75 N or more. When represented by a stress, the strength is, for example, about 10 MPa or more, about 20 MPa or more, and about 25 MPa or more, preferably about 50 MPa or more, and more preferably about 75 MPa or more. Examples of a cell adhesion molecule having a strength which allows for such a support include, but are not limited to, fibronectin, collagen, vitronectin, laminin, integrin, vitronectin, fibrinogen, an immunoglobulin superfamily member, and the like. A typical cell adhesion molecule can be partially altered to enhance the strength thereof (e.g., addition of a substituent). Such an alteration can be achieved by a known method in the art, as described in, for example, “Kobunshikino Zairyo Shirizu Iryokino Zairyo [Molecular Function Series: Medical Functional Material], Kyoritsu Shuppan K. K.; and Guoping Clen et al., J. Biomed. Mater. Res., 51, 273-279, 2000.

In a certain embodiment of the present invention, a support for use in the present invention contains a protein therein. The protein may include those described above (e.g., a cellular adhesive protein, etc.), and preferably, a protein having a strength which allows for the support. Examples of a protein having a strength which allows for the support, include, but are not limited to, fibronectin, collagen, vitronectin, laminin, integrin, vitronectin, fibrinogen, an immunoglobulin superfamily member, and the like. A typical protein can be partially altered to enhance the strength thereof (e.g., complexation with a sugar or lipid, addition of a substituent, etc.). Such an alteration can be achieved by a known method in the art, as described in, for example, “Bunshikino Zairyo Shirizu Iryokino Zairyo [Molecular Function Series: Medical Functional Material], Kyoritsu Shuppan K. K.; and Guoping Clen et al., J. Biomed. mater. Res., 51, 273-279, 2000.

An altered protein or cell adhesion molecule as described above, which is used in the support, is preferably biocompatible.

In a preferred embodiment, a support for use in the present invention may be in the shape of a mesh. In another embodiment, the support may be in the shape of a membrane, a woven, a tube, a sponge, a fiber, or the like. In a certain embodiment, a mesh is preferable. This is because a mesh-like support is easy to coat with a biological molecule. The shape of a support can be appropriately selected depending on the purpose by those skilled in the art. The selected shape can be easily produced based on conventional techniques by those skilled in the art.

A thickness of a support of the present invention may be necessarily changed depending on the purpose. The thickness of the support is preferably about 0.2 mm to about 1.0 mm. When the support is used for a blood vessel or the like, the thickness of the support may preferably be at least about 0.6 mm.

Preferably, in an implant of the present invention, a support may be advantageously coated with a biological molecule. By coating, a biological molecule may be substantially uniformly distributed in the implant. A coating method is known in the art as described in, for example, “Saiseiigaku to Seimeikagaku [Regeneration Medicin and Life Science], Kyoritsu Shuppan; and Guoping Clen, et al., J. Biomed. mater. Res., 51, 273-279, 2000.

In a preferred embodiment, when a support contained in the implant of the present invention has a gap (e.g., in the case of a mesh-like support), the gap may be advantageously filled with a biological molecule. The term “filled” or “blocked” with respect to a gap means that undesired fluid (e.g., liquid or gas) does not pass through the gap. The filled gap can prevent leakage of liquid or gas through the implant. Therefore, the filled gap may be useful for repairing a damaged organ associated with blood, such as blood vessel, heart, or the like.

Preferably, a biological molecule for use in the present invention includes a crosslinking molecule. The crosslinking molecule is crosslinked with a support. Examples of a crosslinking molecule for use in the present invention include, but are not limited to, premature crosslinking (Schiff base crosslink), mature crosslinking (pyridinoline), aging-associated collagen crosslinking (histidinoalanine) collagen, and the like. Preferably, the crosslinking molecule is a mature crosslinking (pyridinoline) collagen.

In a certain embodiment, a support for use in the present invention may include the same material as that for a biological molecule contained in the present invention. In this case, an implant of the present invention may be formed only of the biological molecule. Therefore, for example, an implant of the present invention may be formed of an HGF or collagen. In this case, a certain level of strength needs to be secured. In order to obtain such a strength, the biological molecule may be altered. Such an alteration can be appropriately achieved using a known method in the art by those skilled in the art.

In another embodiment, a cell may be attached to an implant of the present invention. In an embodiment, the present invention is characterized in that an implant without any cell can be cellularized. Even if a cell is attached to an implant, the implant can provide the same effects (in-situ cellularization, repair, etc.) as described herein. Therefore, it should be understood that an implant with a cell falls within the scope of the present invention. This is because even in the case of an implant with a cell, the cell vanishes about one month after implantation and self cells replace and survive.

In one embodiment, a graft of the present invention may be implanted into a body. When the graft is used for implantation, examples of a site targeted by the graft implantation include, but are not limited to, cardiac valve, blood vessel, blood vessel-like tissue, cardiac valve, heart, pericardium, dura mater, skin, bone, soft tissue, trachea, and the like. Preferably, the target site may be blood vessel-like tissue, cardiac valve, heart, pericardium, dura mater, skin, bone, soft tissue, trachea, and the like.

In a certain embodiment, an implant of the present invention may be used for repairing damage on an organ or tissue. An organ or tissue whose damage is targeted may be selected from those described above. Preferably, a target injured site may be heart, liver, kidney, stomach, intestine, brain, bone, trachea, skin, blood vessel, soft tissue, or the like. For the repair purposes, an implant of the present invention preferably has an area greater than or equal to that of the injured site, preferably an area covering the injured site entirely. A desired object may be achieved by an area smaller than that of the injured site. An area substantially covering the injured site can suppress an event accompanying an adverse influence due to damage (e.g., bloodshed, etc.), resulting in an effective therapeutic effect.

In another embodiment, an implant of the present invention may be used for reinforcement of an organ or tissue. For the reinforcement purposes, an implant of the present invention preferably has an area greater than or equal to that of the injured site, preferably an area covering the injured site entirely. A desired object may be achieved by an area smaller than that of the injured site. An area substantially covering the injured site can suppress an event accompanying an adverse influence due to damage (e.g., bloodshed, etc.), resulting in an effective therapeutic effect.

In another embodiment, an implant of the present invention is preferably sterilized. Examples of a sterilization method include, but are not limited to, autoclave sterilization, dry heat sterilization, drug sterilization (e.g., alcohol sterilization, formalin gas or ozone gas sterilization, etc.), radiation sterilization (γ-ray radiation, etc.), and the like. The above-described sterilization can be conducted by, for example, alcohol sterilization, γ-ray radiation, ethyleneoxide gas sterilization, or the like. Therefore, the term “capable of being sterilized” with respect to a material, a support or the like as described herein refers to an ability to resist at least one sterilization method. By sterilization, it is possible to prevent a secondary adverse event, such as infection or the like.

In another preferred embodiment, an implant of the present invention may include an immunosuppressant therein or therewith. Such an immunosuppressant may be known in the art. For the immunosuppression purposes, another method for achieving immunosuppression may be used instead of use of an immunosuppressant. Examples of such an immunosuppression method for avoiding a rejection reaction include, but are not limited to, use of an immunosuppressant, a surgical operation, radiation exposure, and the like. Examples of a representative immunosuppressant include adrenocorticosteroids, cyclosporine, FK506, and the like. Adrenocorticosteroids reduce the number of circulating T-cells to inhibit the nucleic acid metabolism and cytokine production of lymphocytes and prevent the migration and metabolism of macrophages, thereby suppressing an immune reaction. Cyclosporine and FK506 have similar actions in which they bind to a receptor on the surface of a helper T-cell and enter the cell, and directly act on DNA so as to inhibit production of interleukin-2. Eventually, the function of killer T-cells is impaired, resulting in immunosuppression. Use of these immunosuppressants has side effect problems. Particularly, the steroid has a number of side effects. Cyclosporine is toxic to the liver and the kidney. FK506 is toxic to the kidney. Examples of surgical operation to achieve immunosuppression include lymphnodectomy, splenectomy, thymectomy, and the like, whose effects have not been sufficiently revealed. Thoracic duct drainage is a surgical operation, in which circulating lymphocytes are withdrawn to the outside of a body. The effect of this surgical operation has been confirmed, however, this technique has a drawback in that a large amount of serum proteins and fats flow out, potentially leading to nutritional disorder. Radiation exposure includes whole body irradiation and graft irradiation. The effect of radiation exposure is uncertain and puts a large load on recipients. Therefore, radiation exposure is used in combination with the above-described immunosuppressant. None of the above-described techniques is preferable in terms of prevention of a rejection reaction.

An implant of the present invention further comprises an additional medicament component. Preferably, such a medicament component may be advantageously one that does not hinder the aggregating and joining of cells. Alternatively, such a medicament component may be one that has an advantageous action for ameliorating an injured site for the purpose of treatment. Examples of the medicament component include, but are not limited to, heparin, an antibiotic, a vasodilator, an antihypertensive agent (ACE inhibitor, ARB (=ACE receptor blocker)), and the like.

In a preferred embodiment, an implant of a biological molecule for use in the present invention may be advantageously derived from an organism in need of implantation of the implant. As used herein, the term “derived from an organism” with respect to a material means that the material is isolated from the organism or a material is synthesized or replicated from the isolated material. Such a material is also called “self-derived material” or “self material”. By using a self-derived biological molecule, it is possible to prevent immunological rejection efficiently.

In another embodiment, the present invention relates to a medicament containing a biocompatible implant of the present invention. Such a medicament preferably complies with a standard, such as the Japanese Pharmaceutical Affairs Law or the like. Therefore, in this case, a component contained in a biocompatible implant may comply with such a standard. Examples of a component complying with such a standard include, but are not limited to, type I collagen, type IV collagen, and the like. There are various materials which can comply with a standard if application is made to the authority. Therefore, it is only indicated herein that examples described herein have been approved as complying with a standard by an authority. It should be note that the present invention is interpreted as being limited to these examples.

In another embodiment, the present invention relates to a medical kit or system comprising a biocompatible implant of the present invention and instructions indicating a method of using the implant. The instructions describe a method of implanting the implant of the present invention into a predetermined site. Such implantation can be conducted by a method well known in the art as described in, for example, “Shin Gekagaku Taikei, Shinzoishoku•Haiishoku Gijyutsuteki, Rinriteki Seibi kara Jisshi ni Mukete [New Surgery System, Heart Transplantation•Lung Transplantation, From Technical and Ethical Improvement to Implementation]” (Third edition after revision), Hyojyun Gegagaku [Standard Surgery], 9th ed., Igakushoin; and “Shinzo no Geka Shin Gekagaku Taikei [Heart Surgery, New Surgery System]”, 19A, 19B, 19C, (Nakayama Shoten). When an implant of the present invention is implanted by the above-described commonly used method, an excessively large pressure should be preferably avoided.

Examples of a site in which an implant of the present invention is implanted include, but are not limited to, vascular endothelium, vascular smooth muscle, elastic fiber, heart, liver, kidney, stomach, intestine, brain, bone, trachea, skin, blood vessel, soft tissue, and the like. Preferably, such a site is vascular endothelium, vascular smooth muscle, elastic fiber, collagen fiber, or the like.

In a preferred embodiment, instructions accompanying the present invention may describe that a biocompatible implant of the present invention is implanted in such a manner that at least a part of an organ or tissue to be subjected to implantation is left in situ.

Instructions accompanying with the present invention are prepared in accordance with a form defined by an authority in a country in which the present invention is carried out (e.g., the Health, Labor and Welfare Ministry in Japan, the Food and Drug Administration (FDA) in the USA, etc.) and explicitly describe the approval by the authority. Instructions are a so-called “package insert”, which is typically provided as a paper medium. Alternatively, instructions may be provided in the form of an electric medium (e.g., a web site or an electronic mail on the Internet, etc.).

The implant and kit of the present invention are usually used under the supervision of a physician. However, if permitted by the authority and the law in a country, they can be used without the supervision of a physician.

In another embodiment, the present invention provides a method for treating an injured site of a body. The method comprises the step of: A) implanting a biocompatible implant to a part or whole of the injured site, wherein the biocompatible implant comprises: A-1) a biological molecule; and A-2) a support. Here, the implant may make either direct or indirect contact with the injured site. Preferably, in the implanting step of the present invention, the biocompatible implant may be advantageously implanted in such a manner that at least a part of an organ or tissue to which the injured site belongs is left. If a part is left, cells present in the residual tissue may be activated by the biological molecule, resulting in promotion of in-situ cellularization.

In a preferred embodiment, the treatment method of the present invention may further comprise administrating a cellular physiologically active substance. Examples of such a cellular physiologically active substance include, but are not limited to, a granulocyte macrophage colony stimulating factor (GM-CSF), a macrophage colony stimulating factor (M-CSF), a granulocyte colony stimulating factor (G-CSF), a multi-CSF (IL-3), a leukemia inhibiting factor (LIF), a c-kit ligand (SCF), an immunoglobulin family (CD2, CD4, CD8), a platelet derived growth factor (PDGF), an epidermal growth factor (EGF), a fibroblast growth factor (FGF), a hepatocyte growth factor (HGF), a vascular endothelial growth factor (VEGF), and the like.

In a preferred embodiment, the method of the present invention may further comprise performing treatment for suppressing an immune reaction. Methods for suppressing an immune reaction are described above. Preferably, an immunosuppressant may be advantageously used in combination with the method.

In another embodiment, the present invention provides a method for reinforcing an organ or tissue in a body. The method comprises the step of: A) implanting a biocompatible implant to a part or whole of the organ or tissue, wherein the biocompatible implant comprises: A-1) a biological molecule; and A-2) a support. Such implantation can be conducted by a method well known in the art, which is used as it is or in an appropriately adapted from, as described in, for example, “Shin Gekagaku Taikei, Shinzoishoku•Haiishoku Gijyutsuteki, Rinriteki Seibi kara Jisshi ni Mukete [New Surgery System, Heart Transplantation•Lung Transplantation, From Technical and Ethical Improvement to Implementation]” (Third edition after revision).

In another embodiment, the present invention provides a method for producing or regenerating an organ or tissue. The method comprises the steps of: A) implanting a biocompatible implant to a part or whole of the organ or tissue within an organism containing the organ or tissue, wherein the biocompatible implant comprises: A-1) a biological molecule; and A-2) a support; and B) culturing the organ or tissue within the organism.

In the method for producing or regenerating an organ or tissue, the implanting step can be conducted as described above. The culturing step can be conducted by keeping organisms under usual conditions. The conditions are well known in the art and can be appropriately established by those skilled in the art, taking into consideration the type, size or the like of the organism.

In another embodiment, the present invention relates to use of a biocompatible graft of the present invention for treatment of an injured site within a body. In the use, the biocompatible graft may be employed in any form described herein.

In another embodiment, the present invention relates to use of a biocompatible graft of the present invention for reinforcement of an organ or tissue within a body. In the use, the biocompatible graft may be employed in any form described herein.

In another embodiment, the present invention relates to use of a biocompatible graft of the present invention for production of a medicament for treating an injured site within a body. In the use, the biocompatible graft may be employed in any form described herein.

In another embodiment, the present invention relates to use of a biocompatible graft of the present invention for production of a medicament for reinforcing an organ or tissue within a body. In the use, the biocompatible graft may be employed in any form described herein.

Methods for producing medicaments are well known in the art. The medicament of the present invention may be prepared for storage by mixing a sugar chain composition having the desired degree of purity with optional physiologically acceptable carriers, excipients, or stabilizers (Japanese Pharmacopeia 14th Edition or the latest edition: Remington's Pharmaceutical Sciences, 18th Edition, A. R. Gennaro, ed., Mack Publishing Company, 1990; and the like), in the form of lyophilized cake or aqueous solutions.

A pharmaceutically acceptable carrier contained in a medicament of the present invention includes any material known in the art. Examples of such a pharmaceutically acceptable carrier include, but are not limited to, antioxidants, preservatives, colorants, flavoring agents, diluents, emulsifiers, suspending agents, solvents, fillers, bulking agents, buffers, delivery vehicles, agricultural or pharmaceutical adjuvants, and the like. In the case of a medicament of the present invention, representatively, a support and a biological molecule are administered with at least one physiologically acceptable carrier, excipient or diluent in the form of a composition. Examples of an appropriate vehicle may include an injection solvent, a physiological solution, or artificial cerebrospinal fluid. Other materials which are commonly used in a composition for implantation can be added to the above-described materials.

Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor.

Acceptable carriers, excipients or stabilizers used herein preferably are nontoxic to recipients and are preferably inert at the dosages and concentrations employed, and preferably include phosphate, citrate, or other organic acids: ascorbic acid, α-tocopherol; low molecular weight polypeptides; proteins (e.g., serum albumin, gelatin, or immunoglobulins); hydrophilic polymers (e.g., polyvinylpyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

(Composite Support)

In another embodiment, the present invention provides a biocompatible tissue support having a novel structure. This support comprises: A) a first layer having a rough surface; and B) a second layer having a strength which allows the second layer to resist in vivo impact, wherein the first layer is attached to the second layer via at least one point. The support is implanted into an organism and is used as a scaffold for compensating for an organ. Here, the first layer having a rough surface is typically used as an internal layer when the support is applied to organisms.

In one embodiment of the present invention, typically, a knit is used as the first layer having a rough surface. Any biocompatible material capable of being knitted may be used as a material for a knit. A knit can be produced by any known method in the art. A knit can be produced by preparing a thread-like material, making loops with the thread, and joining the loops successively. In a knit, there is a gap between each loop, which provides a sufficient space for accommodating a cell(s). A knit provides a layer having a thickness greater than that of a woven.

In one embodiment of the present invention, a woven is used as the second layer of a support of the present invention. Any biocompatible material capable of being woven may be used as a material for a woven. Any method for producing a woven, which is known in the art, can be used. A woven can be produced by interlacing the threads of the warp and the weft, for example. There is substantially no gap in a woven, whereby liquid (e.g., body fluid, such as blood) is not likely to leak.

The present invention also provides a structure comprising biocompatible knit and woven implant layers and an intermediate layer for attaching the knit layer with the woven layer. This structure can unexpectedly solve both the leakage problem with knit and the fray problem with woven. The combination of knit and woven also unexpectedly provides a material which has space for accommodating cells while preventing leakage and fray. In addition, by providing a biological molecule (e.g., collagen, etc.) to the support, when the support is placed in organisms, cells aggregate to the support in the early period and subsequently the support itself is biologically degraded and eventually vanishes. Thereby, a graft which leaves substantially no trace can be provided. Preferably, the first layer used as an internal layer advantageously has a higher rate of biodegradation than that of the second layer, though the present invention is not limited to this. By selecting any method to produce a knit and a woven, the composite support is given a predetermined strength and a predetermined thickness. The absorption rates of a knit and a woven can be controlled by selecting any materials for threads used in the knit and the woven. Further, a support suited to the regeneration rate of a tissue and having a required strength can be produced. Thus, the present invention is considered to be used in various applications.

In one embodiment, the rough surface of the first layer in the present invention has a sufficient space for accommodating cells. By providing sufficient accommodation for cells, cells are easily attached to the layer and survive after the implant is implanted. Alternatively, cells may be provided in a support of the present invention before implantation. In this case, the above-described space is useful for carrying the cells.

In a preferred embodiment, a support of the present invention has an intermediate layer. With the intermediate layer, the first layer and the second layer can be efficiently attached or sealed together.

In one embodiment, the attachment of the intermediate layer is achieved by melting a biological absorbable macromolecule. The attachment can be carried out by any means. For example, a material having a melting point lower than the melting point of layers to be attached together is used as the intermediate layer, i.e., a difference in melting point is utilized. The attachment can be achieved by heating the structure to a temperature which is higher than the melting point of the intermediate layer material and is lower than the melting points of the other layer materials. Alternatively, a biological material, such as fibrin, is used as a glue. The intermediate layer is preferably is in the form of a film, though the present invention is not limited to this.

In a preferred embodiment, the second layer of the present invention has substantially no permeability to air. The lack of air permeability can be confirmed by a water leakage test.

The strength of a support of the present invention may be typically at least about 10 N, more preferably about at least 20 N, even more preferably at least about 50 N, and still even more preferably at least 100 N, where the strength is represented by a force measured by a tension test. When represented by a stress, the strength may be, for example, about 10 MPa or more, about 20 MPa or more, and about 25 MPa or more, preferably about 50 MPa or more, and more preferably about 75 MPa or more.

A support of the present invention usually has a modulus of elasticity of 100 MPa or less, preferably about 80 MPa or less. A support of the present invention may have a modulus of elasticity less than a naturally-occurring material as long as it can withstand use. A support of the present invention usually has a strain rate of at least 105%, preferably 110%. The strain is measured both in the longitudinal direction and in the transverse direction. Preferably, there is substantially no variation in the strains in both of the directions, though the present invention is not limited to this. The strain is preferably at least 120% or 150% depending on the application, though the present invention is not limited to this. A support of the present invention may have a strain lower than that of a naturally-occurring material as long as it can withstand use.

In one embodiment, a support of the present invention usually has an air permeability of 25 ml/cm²/sec or less, more usually 15 ml/cm²/sec or less, preferably 10 ml/cm²/sec or less, more preferably about 5 ml/cm²/sec or less, more preferably about 4 ml/cm²/sec or less, and even more preferably about 3 ml/cm²/sec or less. A conventional structure consisting only of a knit and a woven achieves about 5 ml/cm²/sec at best. In contrast, a support of the present invention unexpectedly has an air permeability higher than that value. The air permeability of a support can be herein measured in accordance with JIS-L-1096A. Specifically, a test piece is attached to a Frazil-type Air Permeability Tester. A pressurize resister is used to adjust pressure to 125 Pa while measuring the pressure inclined-type barometer. The amount of passing air (ml/cm²/sec) is measured to determine an air permeability. Air permeability is associated with a water leakage rate, and therefore, may be represented by a water leakage rate. In this case, a water leakage rate is advantageously 5 ml or less per 10 ml per 60 sec, preferably 3 ml or less, more preferably 2 ml or less, and more preferably 1 ml or less.

In one embodiment, the first layer and/or the second layer of a support of the present invention include a biodegradable material selected separately. Preferably, both the first layer and the second layer have a biodegradable material. The degradation rate of the biodegradable material is a period of time which is sufficient for cells to be accepted (e.g., several months).

Such a biodegradable material may be at least one component selected from the group consisting of poly(glycolic acid) (PGA), poly(L-lactic acid) (PLA) and polycaprolactum (PCLA) and a copolymer thereof, or a mixture thereof. Alternatively, the biodegradable polymer may contain PLGA having a glycolic acid-to-lactic acid ratio of about 90 about 10 to about 80: about 20.

In a preferred embodiment, the first layer of a support of the present invention contains poly(glycolic acid). This is because it is easy to produce the first layer as a knit. In addition, the acceptance of cells is satisfactory.

In another preferred embodiment, the second layer of a support of the present invention contains poly(L-lactic acid). This is because it is easy to produce the second layer as a woven. In addition, the acceptance of cells is satisfactory.

In a preferred embodiment of the present invention, the second layer is a woven while the first layer is a knit. With such a structure, a support can have improved the cell acceptance ability and hold a satisfactory strength. No support having such a structure has been achieved. Thus, the present invention provides an effect which cannot be obtained by conventional supports. By combining such a composite support with a biological molecule, such as collagen, laminin or the like, cell acceptance can be further improved, thereby making it possible to enhance a function of regenerating and repairing.

In a preferred embodiment, a support of the present invention comprises a second layer which is a woven of poly(L-lactic acid) and a first layer which is a knit of poly(glycolic acid). With such a structure, the strength can be retained, leakage is prevented, a space for a biological molecule (e.g., collagen) can be accommodated, a predetermined thickness is given to the support, fray is prevented, and the strength and the thickness can be controlled. Thus, the effects which cannot be achieved by conventional supports can be achieved. For example, a support having a conventional woven structure can retain strength but does not have cell acceptance ability.

In one embodiment, the intermediate layer contains a synthetic biological absorbable polymer. The polymer is preferably a poly(lactic acid) film or a caprolactam film. Such a film has a low melting point and is easy to adhere, and is thus easy to produce. Therefore, in a preferred embodiment, a material for the intermediate layer has a melting point lower than at least one, or preferably both, of the melting points of the first layer and the second layer.

The first layer and the second layer may comprise only one layer or a plurality of layers. In a preferred embodiment, the first layer comprises a plurality of knit layers. In another preferred embodiment, the second layer comprises a plurality of woven layers. The first layer may comprise another layer (e.g., a knit) in addition to the knit layer.

In another preferred embodiment, the first layer is provided with a biological molecule. In this embodiment, any embodiment described in relation to the above-described biological molecule-attached implant may be used. Preferably, the biological molecule is an extracellular matrix. Particularly, a preferable biological molecule includes an extracellular matrix selected from the group consisting of collagen and laminin.

The biological molecule is preferably provided in such a manner that the biological molecule is contained in a microsponge. Such a microsponge is preferable since it has a form suitable for scaffolding cells.

Preferably, the biological molecule is advantageously crosslinked with a support. When collagen is used, the crosslinking is carried out by collagen crosslink treatment.

In another embodiment, the present invention provides a medicament comprising a support of the present invention. The support contained in the medicament may have the form of any of the above-described supports. The support of the present invention is characterized in that the support does not need to contain cells. In another embodiment, the medicament of the present invention may contain a cell.

In one embodiment, a medicament of the present invention is used for implantation into a body. It has been found that after the medicament is implanted, cells are accepted by the support. Such an effect was not expected from conventional supports. After several weeks to several months, cells form a tissue to repair an implanted portion. In a preferred embodiment, since the medicament is made of a biodegradable material, the material itself vanishes before or after the implanted portion is repaired. Thus, a support of the present invention can advantageously repair an implanted portion to a perfectly natural state. Such an effect cannot be achieved by conventional supports, patches or the like.

In a particular embodiment, examples of a site to which a support of the present invention is implanted include, but are not limited to, heart, cardiac valve, blood vessel, pericardium, cardiac septum, intracardiac conduit, extracardiac conduit, dura mater, skin, bone, soft tissue, trachea, and the like. Preferably, a support of the present invention is applied to a portion through which liquid (e.g., blood) flows. Examples of such a portion include, but are not limited to, digestive tract, blood vessel, heart, cardiac valve, and the like.

In a preferred embodiment, a biological molecule for use in a medicament of the present invention may be advantageously derived from an organism undergoing implantation, though the present invention is not limited to this. Such a biological molecule derived from the same origin as a host is considered to be substantially free from an immune reaction or the like, and therefore, is considered to be advantageous. Note that if a biological molecule is purified, it is considered not to elicit an immune reaction. Therefore, the origin of the biological molecule is not particularly limited.

(Production Method for Support)

In another embodiment, the present invention provides a method for producing a biocompatible tissue support, wherein the biocompatible tissue support comprises: A) a first layer having a rough surface; and B) a second layer having a strength which allows the second layer to resist in vivo impact, wherein the first layer is attached to the second layer via at least one point. The method comprises the step of: attaching the first layer with the second layer. The attaching step is carried out by, for example, an ultrasonic sewing machine, UV light, or the like, though the present invention is not limited to these.

The ultrasonic sewing machine is known in the art. Examples of the ultrasonic sewing machine include, but are not limited to, a commercially available ultrasonic sealer (e.g., an arm type (e.g., US-1150 manufactured by Brother), a CNC type (US-7010), a unit type (US-2150) (available from Brother, Aichi, Japan)).

In one embodiment, the method comprises a) providing the intermediate layer between the first layer and the second layer: b) providing the first layer, the second layer and the intermediate layer under conditions that the first layer and the second layer are not melted and the intermediate layer is melted; and c) the intermediate layer is provided under conditions that the intermediate layer is solidified, while retaining desired shapes of the first layer, the second layer and the intermediate layer.

In a preferred embodiment, the melting point of the intermediate layer is lower than any one or both of the melting points of the first layer and the second layer and a difference between the melting points is utilized.

In a preferred embodiment, the second layer is a woven of poly(L-lactic acid) and the first layer is a kit of poly(glycolic acid), and the intermediate layer is a lactic acid film or a caprolactam film. The melting point of the intermediate layer is advantageously from 80° C. to 140° C., preferably from 100° C. to 140° C.

In another preferred embodiment, when the intermediate layer is made of caprolactam, the melting point thereof is advantageously from about 80° C. to 140° C. When the attachment is conducted at such a temperature, the attachment strength is significantly improved as compared to other temperatures by a factor of two or more. Therefore, a preferred temperature is higher than the melting point of a material for the intermediate layer and lower than the melting points of materials for the first layer and the second layer.

In another embodiment, in a production method for a support of the present invention, the support of the present invention further comprises a biological molecule. In this case, the method of the present invention further comprises attaching the biological molecule to the first layer. Such an attaching step can be carried out by any technique, and preferably comprises crosslink treatment.

In one embodiment, a biological molecule for use in a support of the present invention is collagen. In this case, the attaching step comprises collagen crosslink treatment.

In one embodiment, the intermediate layer of a support of the present invention is produced by casting a film material onto a glass plate, followed by air drying, to form a film. Such a film is suitable for sealing, and therefore, is preferably used for production of a support of the present invention.

In one embodiment, the attaching step of the present invention preferably comprises exerting a pressure of at least about 0.1 g/cm² onto the support, more preferably, at least about 0.5 g/cm², and even more preferably 0.75 g/cm².

(Therapeutic Method)

In another embodiment, the present invention provides a method for treating an injured site of a body, comprising the step of: A) implanting a biocompatible tissue support to a part or whole of the injured site, wherein the biocompatible tissue support comprises: A-1) a first layer having a rough surface; and A-2) a second layer having a strength which allows the second layer to resist in vivo impact, wherein the first layer is attached to the second layer via at least one point. Here, the implant may be made either direct or indirect contact with the injured site. Preferably, in the implanting step of the present invention, the biocompatible implant may be advantageously implanted in such a manner that at least a part of an organ or tissue to which the injured site belongs is left. If a part is left, cells present with in the residual tissue may be activated by the biological molecule, resulting in promotion of in-situ cellularization. The biocompatible tissue support for use in the method for treating an injured site of the present invention may be selected from those described above.

In a preferred embodiment, the treatment method of the present invention may further comprise administering a cellular physiologically active substance. Examples of such a cellular physiologically active substance include, but are not limited to, a granulocyte macrophage colony stimulating factor (GM-CSF), a macrophage colony stimulating factor (M-CSF), a granulocyte colony stimulating factor (G-CSF), a multi-CSF (IL-3), a leukemia inhibiting factor (LIF), c-kit ligand (SCF), an immunoglobulin family (CD2, CD4, CD8), a platelet derived growth factor (PDGF), an epidermal growth factor (EGF), a fibroblast growth factor (FGF), a hepatocyte growth factor (HGF), a vascular endothelial growth factor (VEGF), and the like.

In a preferred embodiment, the method of the present invention may further comprise treatment for suppressing an immune reaction. Such treatment for suppressing an immune reaction is described above. In this case, preferably, an immunosuppressant is advantageously used.

In another embodiment, the present invention provides a method for reinforcing an organ or tissue within a body, comprising the step of: A) implanting a biocompatible tissue support to a part or whole of the injured site, wherein the biocompatible tissue support comprises: A-1) a first layer having a rough surface; and A-2) a second layer having a strength which allows the second layer to resist in vivo impact, wherein the first layer is attached to the second layer via at least one point. Such implantation can be conducted by a method well known in the art, which is used as it is or in an appropriately adapted form, as described in, for example, “Shin Gekagaku Taikei, Shinzoishoku•Haiishoku Gijyutsuteki, Rinriteki Seibi kara Jisshi ni Mukete [New Surgery System, Heart Transplantation•Lung Transplantation, From Technical and Ethical Improvement to Implementation]” (Third edition after revision). The biocompatible tissue support for use in the method for treating an injured site of the present invention may be selected from those described above.

In another embodiment, the present invention provides a method for producing or regenerating an organ or tissue. The method comprises the steps of: A) implanting a biocompatible tissue support to a part or whole of the organ or tissue within an organism containing the organ or tissue, wherein the biocompatible tissue support comprises: A-1) a first layer having a rough surface; and A-2) a second layer having a strength which allows the second layer to resist in vivo impact, wherein the first layer is attached to the second layer via at least one point; and B) culturing the organ or tissue in the organism. The biocompatible tissue support for use in the method for treating an injured site of the present invention may be selected from those described above.

In the method for producing or regenerating an organ or tissue, the implanting step can be conducted as described above. The culturing step can be conducted by keeping organisms under usual conditions. The conditions are well known in the art and can be appropriately established by those skilled in the art, taking into consideration the type, size or the like of the organism.

In another embodiment, the present invention relates to use of a biocompatible tissue support for treatment of an injured site within a body, wherein the biocompatible tissue support comprises: A-1) a first layer having a rough surface; and A-2) a second layer having a strength which allows the second layer to resist in vivo impact, wherein the first layer is attached to the second layer via at least one point. In this use, the biocompatible tissue support for use in the method for treating an injured site of the present invention may be selected from those described above.

In another embodiment, the present invention relates to use of a biocompatible tissue support for reinforcement of an organ or tissue within a body, wherein the biocompatible tissue support comprises: A-1) a first layer having a rough surface; and A-2) a second layer having a strength which allows the second layer to resist in vivo impact, wherein the first layer is attached to the second layer via at least one point. In this use, the biocompatible tissue support for use in the method for treating an injured site of the present invention may be selected from those described above.

In another embodiment, the present invention relates to use of a biocompatible tissue support for production of a medicament for treatment of an injured site within a body, wherein the biocompatible tissue support comprises: A-1) a first layer having a rough surface; and A-2) a second layer having a strength which allows the second layer to resist in vivo impact, wherein the first layer is attached to the second layer via at least one point. In this use, the biocompatible tissue support for use in the method for treating an injured site of the present invention may be selected from those described above.

In another embodiment, the present invention relates to use of a biocompatible tissue support for production of a medicament for reinforcement of an organ or tissue within a body, wherein the biocompatible tissue support comprises: A-1) a first layer having a rough surface; and A-2) a second layer having a strength which allows the second layer to resist in vivo impact, wherein the first layer is attached to the second layer via at least one point. In this use, the biocompatible tissue support for use in the method for treating an injured site of the present invention may be selected from those described above.

Hereinafter, the present invention will be described by way of examples. Examples described below are provided only for illustrative purposes. Accordingly, the scope of the present invention is not limited except as by the appended claims.

### EXAMPLES

Reagents, materials, and the like used in the examples below were available from Wako Pure Chemical Industries, Sigma, Becton Dicinson and PeptoTech unless otherwise specified.

### Example 1

### Experiment with PLGA

In Example 1, PLGA was used as a support and type I collagen and type IV were used as biological molecules to prepare an implant. As a result, the effect of the present invention was demonstrated.

(Methods and Results)

Ex Vivo Experiment

<Design of Scaffold>

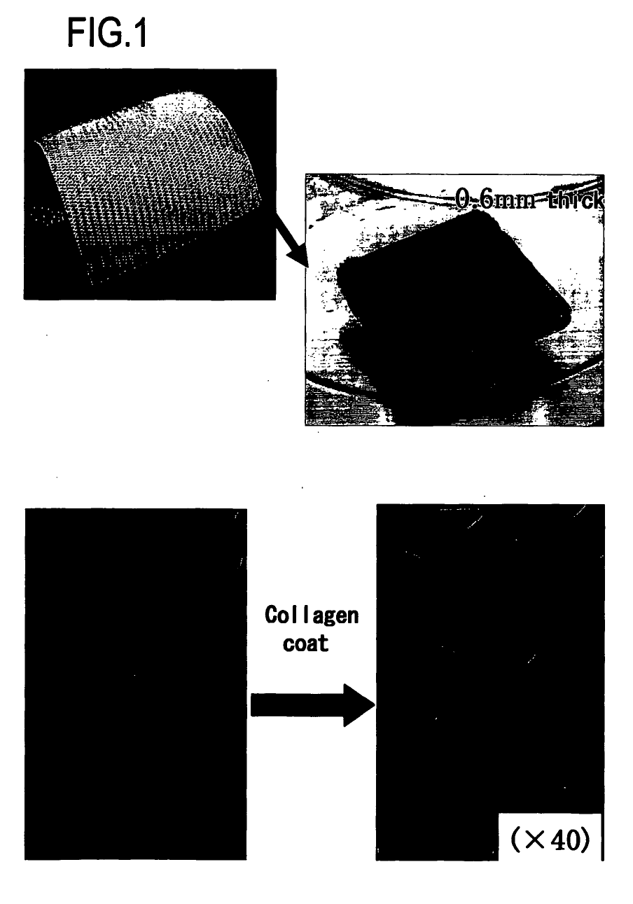

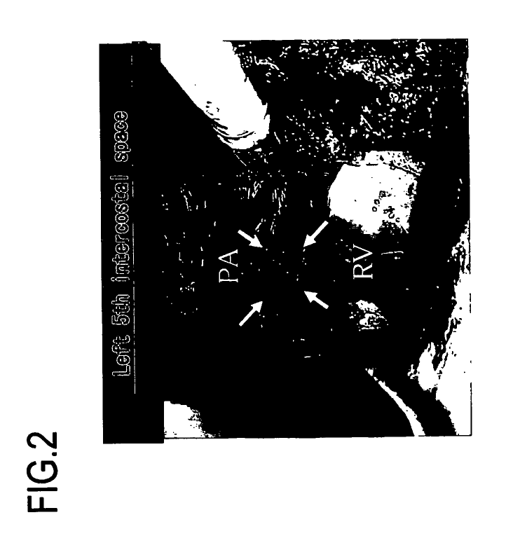

A sheet of knitted mesh was attached to two sheets of woven mesh (0.2 mm thick for each, a total of 0.6 mm thick). When a resultant patch is implanted into an organism, the knit faces the lumen side thereof while the woven faces the outside thereof. These three sheets of mesh were made of a Vicryl polylatin 910 mesh (PLGA (a copolymer having a glycolic acid-to-lactic acid ratio of 90:10)), which is a biodegradable synthetic macromolecule. The resultant structure was subjected to collagen crosslink treatment to obtain a PLGA-collagen composite film which was used as a scaffold. Two groups of scaffolds were prepared: A) only type I collagen was used as a crosslinking agent in crosslink treatment; and B) type I collagen and type IV collagen were used (FIG. 1). A crosslinking method will be described below. FIG. 2 shows a state in which a left 5th intercostal space thoracotomy was used. A 20 mm-diameter patch was stitched to the pulmonary artery trunk.

<Crosslinking Method>

Collagen type IV or laminin was added to a solution containing an extracellular matrix (e.g., type I collagen, type IV collagen, laminin, etc.) to a final concentration of 1/10, if required. The above-described support was impregnated with the solution, followed by lyophilization. A crosslink treatment was conducted for about 4 hours using 37° C. glutaraldehyde saturated vapor. Finally, the support was shaked in 0.1-M aqueous glycine solution for 15 min 3 times, followed by washing with distilled water 3 times, and followed by lyophilization. With this procedure, various extracellular matrix-containing supports were prepared.

<Mechanical Strength>

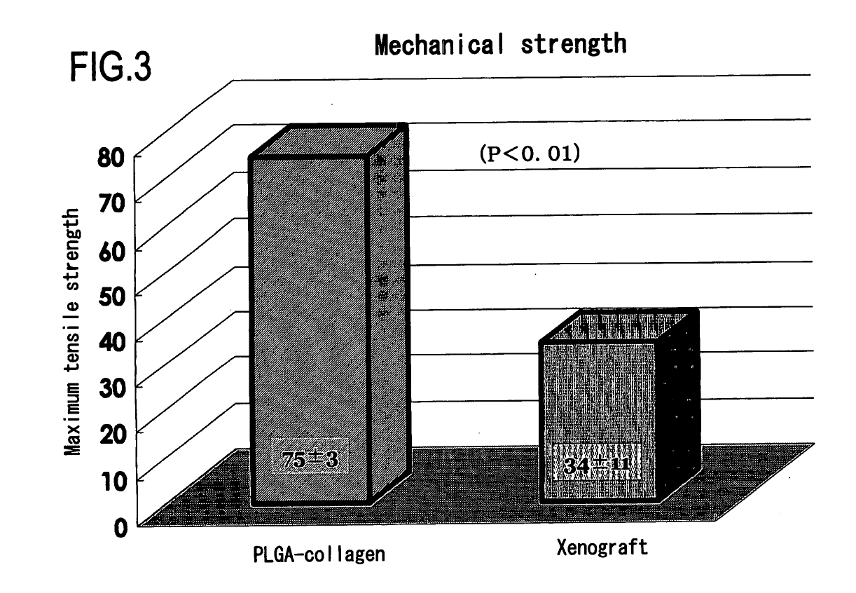

The strength of the PLGA-collagen composite film was measured using a tension tester. A weight was loaded on a strip material having a width of 5 mm and a length of 30 mm in a minor axis direction at a rate of 10 mm/min so as to measure the strain at break and the modulus of elasticity thereof (TENSILLON ORIENTEC). As a control, a glutaraldehyde-treated horse pericardium was used for comparison. The PLGA-collagen composite film had a tension strength of 75±5 N, while the glutaraldehyde-treated horse pericardium had a tension strength of 34±11 N. Thus, the PLGA-collagen composite film has a higher tension strength (FIG. 3).

<Efficiency of Cell Adhesion>

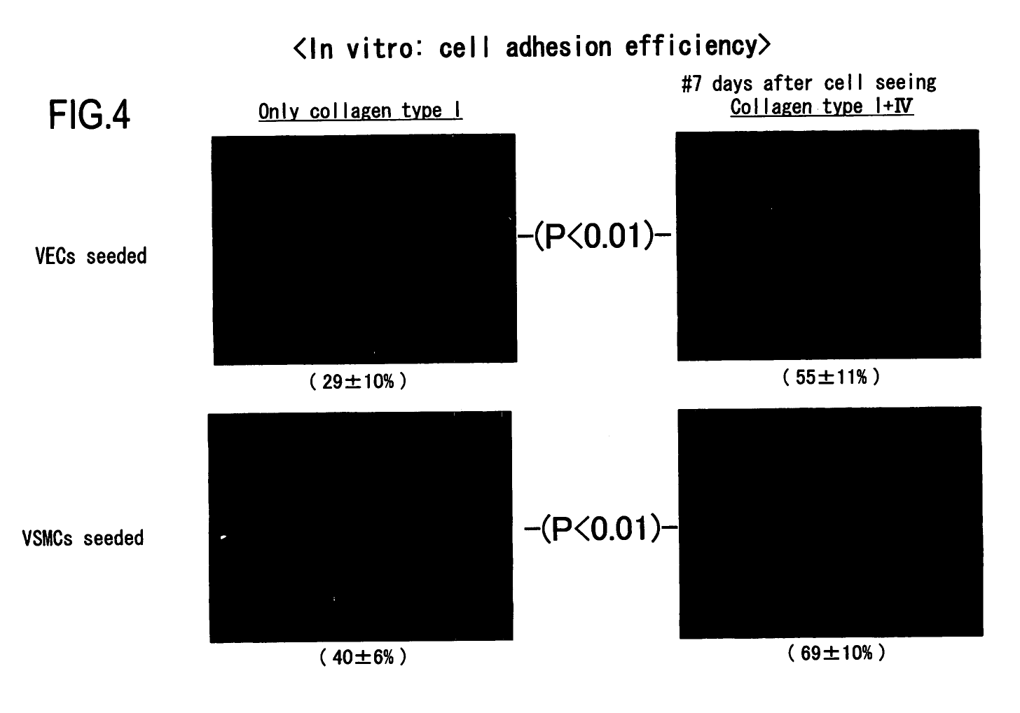

The cell acceptance ability of the PLGA-collagen composite film was determined as follows. The cell adhesion efficiency of vascular endothelial cells (VECs) and vascular smooth muscle cells (VSMCs) labeled with a fluorescent antibody (PKH-26 (SIGMA)) in vitro was compared between a PLGA-collagen composite film subjected to crosslink treatment with only type I collagen and a PLGA-collagen composite film subjected to crosslink treatment with type I and type IV collagen. The cell adhesion efficiency was determined by the color development area (%) of a fluorescent pigment per visual field of a fluorescence microscope. For both vascular endothelial cells (VECs) and vascular smooth muscle cells (VSMCs), the PLGA-collagen composite film subjected to type I and type IV collagen crosslink treatment exhibited a significantly larger color development area of the fluorescent pigment, and cell acceptance was confirmed (FIG. 4).

According to the above-described result, the PLGA-collagen composite film subjected to type I and type IV collagen crosslink treatment had a strength greater than or equal to that of the conventional glutaraldehyde-treated horse pericardium and had a high level of cell acceptance ability. Next, the PLGA-collagen composite film was used to study an in vivo effect of cell seeding before implantation.

<Factor VIII Staining>

The number of blood vessels can be determined by immunohistochemically staining blood vessels with a Factor VIII-relevant antigen or the like and counting the stained blood vessels. Specifically, specimens are fixed with 10% buffered formalin, followed by paraffin embedding. Several continuous slices are prepared from each specimen, followed by freezing. Next, the frozen slice is fixed with 2% paraformaldehyde in PBS for 5 min at room temperature and is immersed in methanol containing 3% hydrogen peroxide for 15 min, followed by washing with PBS. This sample is covered with bovine serum albumin solution for about 10 min to block a non-specific reaction. The specimen is coupled with HRP, followed by incubation overnight with an EPOS-conjugated antibody for the Factor VIII-relevant antigen. After the sample is washed with PBS, the sample is immersed in diaminobenzidine solution (e.g., 0.3 mg/ml diaminobenzidine in PBS) to obtain positive staining. Stained vascular endothelial cells are counted under, for example, an optical microscope (×200 magnification). For example, the result of counting is represented by the number of blood vessels per square millimeters. After a specific treatment, it is determined whether or not the number of blood vessels statistically significantly increased, so as to confirm the presence of Factor VIII. Thereby, for example, the presence and angiogenesis activity of vascular endothelial cells can be determined.

<Elastica van Gieson Staining>

Elastic fiber was stained by elastica van Gieson staining. The procedure is described as follows. A sample is optionally deparaffinized (e.g., with pure ethanol), followed by washing with water. The sample is immersed in resorcin fuchsin solution (available from Muto Chemical, etc.) for 40 to 60 min. Thereafter, the sample is washed with 70% alcohol and is immersed in Omni's hematoxylin for 15 min. Thereafter, the sample is washed with running water for 5 min and is immersed in van Gieson solution for 2 min. The sample is washed, immediately followed by dehydration, clearing, and mounting.

<Hematoxylin•Eosin (HE) Staining>

The fixation or vanishment of cells in a support was observed by HE staining. The procedure is described as follows. A sample is optionally deparaffinized (e.g., with pure ethanol), followed by washing with water. The sample is immersed in Omni's hematoxylin for 10 min. Thereafter, the sample is washed with running water, followed by color development with ammonia water for 30 sec. Thereafter, the sample is washed with running water for 5 min and is stained with eosin hydrochloride solution for 2 min, followed by dehydration, clearing, and mounting.

<Von Kossa Staining>

Cells were stained by von Kossa method in order to observe the calcification in the cell. The procedure is described as follows. A sample is optionally deparaffinized (e.g., with pure ethanol), followed by washing with water (distilled water). The sample is immersed in 25% silver nitrate solution (under indirect light) for 2 hours. Thereafter, the sample is washed with distilled water and is immersed in 42% sodium thiosulfate (hypo) for 5 min. Thereafter, the sample is washed with running water for 5 min and is immersed in Nuclear fast Red (van Gieson) for 5 min. Thereafter, the sample is washed with running water for 5 min, followed by dehydration, clearing, and mounting.

<Implantation>

A PLGA-collagen composite film (15×10 mm) subjected to type I and type IV collagen crosslink treatment and a film obtained by seeding onto such a composite film self vascular endothelial cells (VECs) and self vascular smooth muscle cells (VSMCs), were prepared. These films were implanted into the pulmonary artery trunk of adult beagle dogs (8 to 10 kg) under partial clamping.

The cells were prepared as follows. A vein was extracted from the anterior surface of a lower limb of an adult beagle dog of the same type. Vascular endothelial cells (VECs) and vascular smooth muscle cells (VSMCs) were isolated from the vein, followed by culture. The vascular endothelial cells and the vascular smooth muscle cells were seeded onto the PLGA-collagen composite film at 1.3×10⁶ cells/cm², respectively. After implantation, the film was removed and histologically examined after two weeks, two months, and 6 months.

<In Vivo: Two Weeks After Implantation>

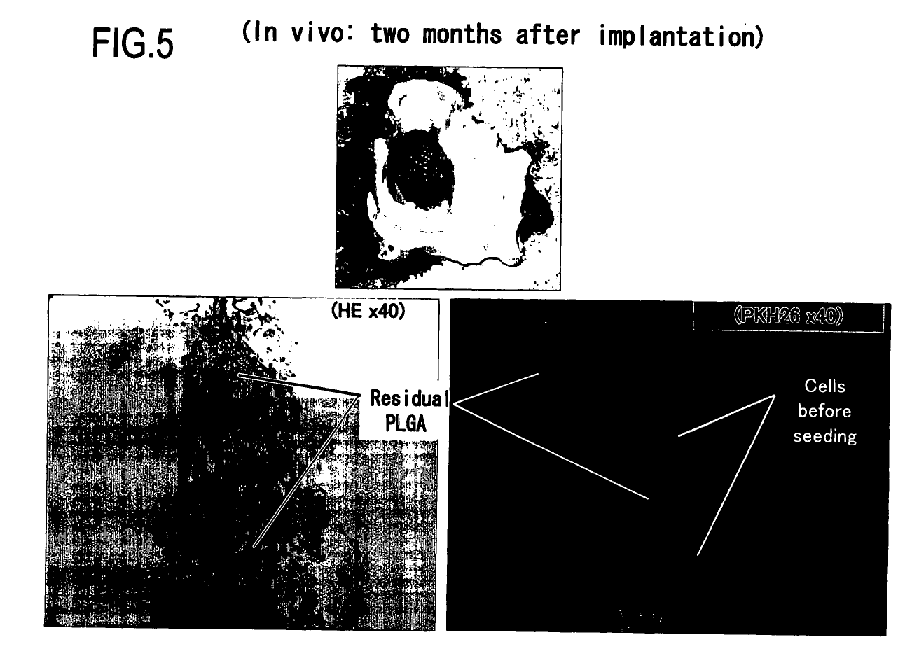

For both the PLGA-collagen composite film and the self cell-seeded PLGA-collagen composite film, no clear thrombus formation was confirmed using the naked eye. In the case of HE staining, PLGA residues were observed and connective tissue was present therebetween. In the PLGA-collagen composite film having the seeded self vascular endothelial cells or vascular smooth muscle cells, only seeded fluorescent antibody-labeled vascular endothelial cells were scattered on the internal side of the film. Therefore, it is suggested that most of the cells were detached from the PLGA-collagen composite film (FIG. 5).

<In Vivo: Two Months After Implantation>

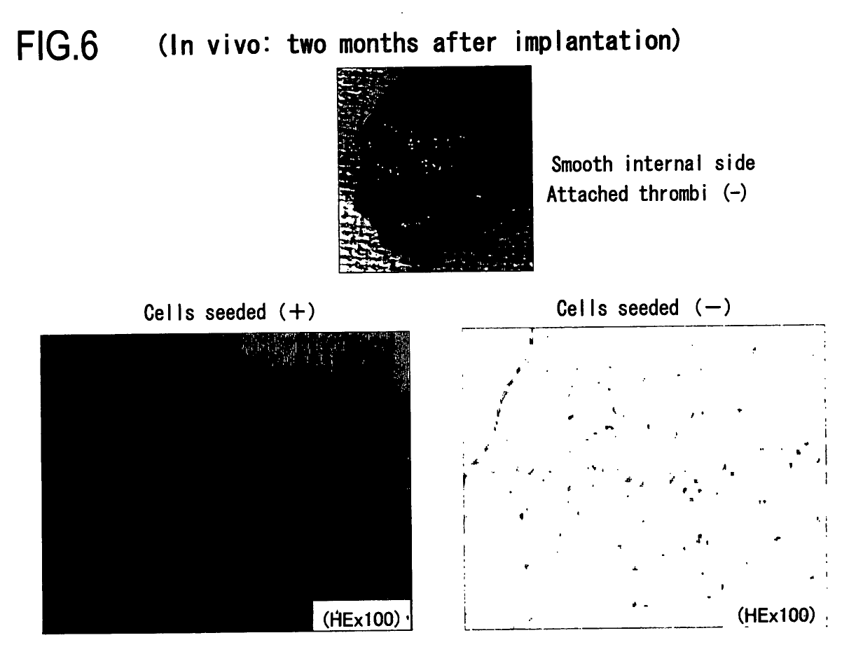

Both the PLGA-collagen composite film and the self cell-seeded PLGA-collagen composite film had a smooth internal surface observed with the naked eye. HE staining indicated complete absorption of PLGA and a tissue structure comparable to normal blood vessels (FIG. 6).

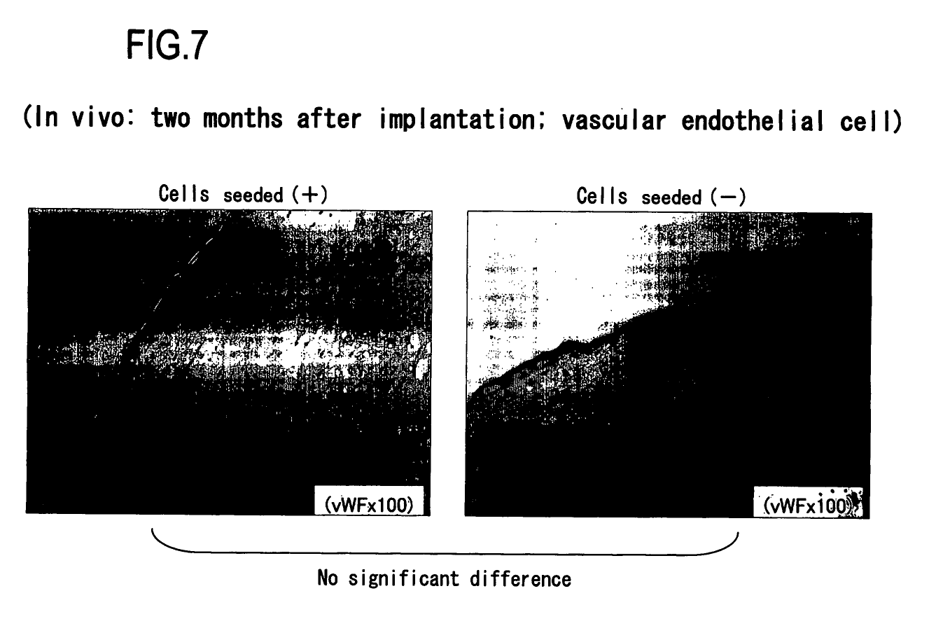

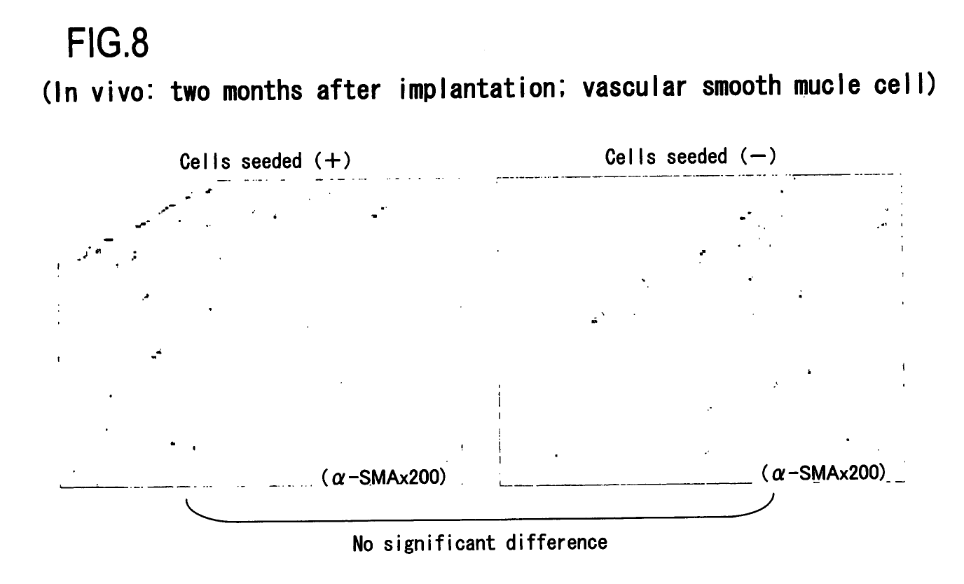

The vascular endothelial cells were studied by Factor VIII staining and the vascular smooth muscle cells were studied by α-SMA (smooth muscle actin) immunostaining. In both of the films, Factor VIII immunostaining indicated a monolayer of continuous vascular endothelial cells (FIG. 7) and the α-SMA immunostaining indicated the smooth muscle cells aligned on the internal surface (FIG. 8).

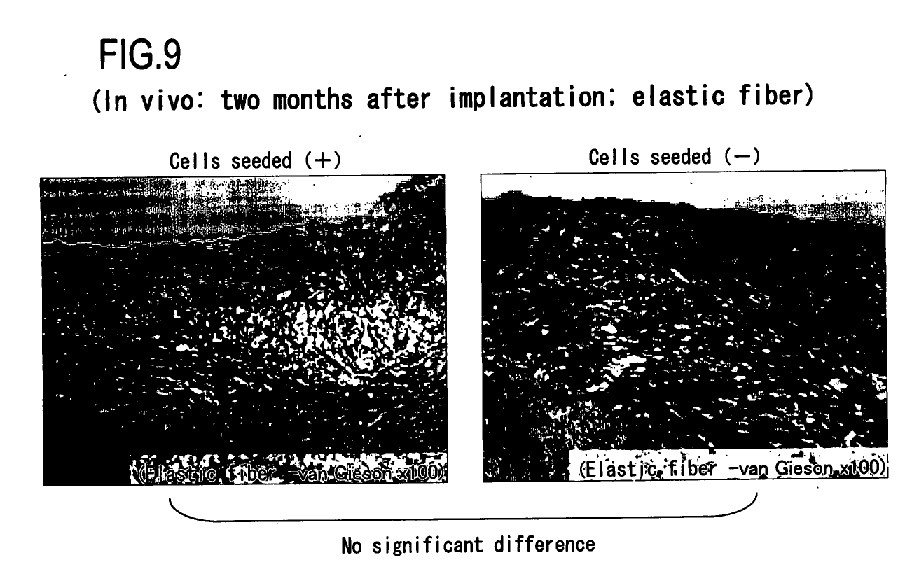

Moreover, the vascular elastic fiber was studied by elastica van Gieson staining. In both of the films, elastic fiber was observed in an internal layer of a blood vessel (FIG. 9).

<In Vivo: Six Months After Implantation>

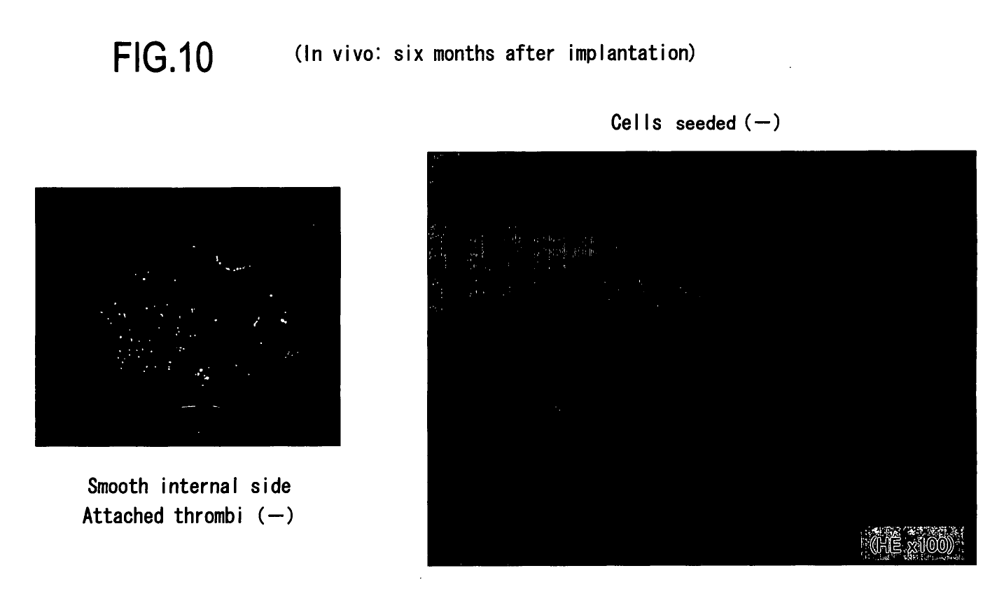

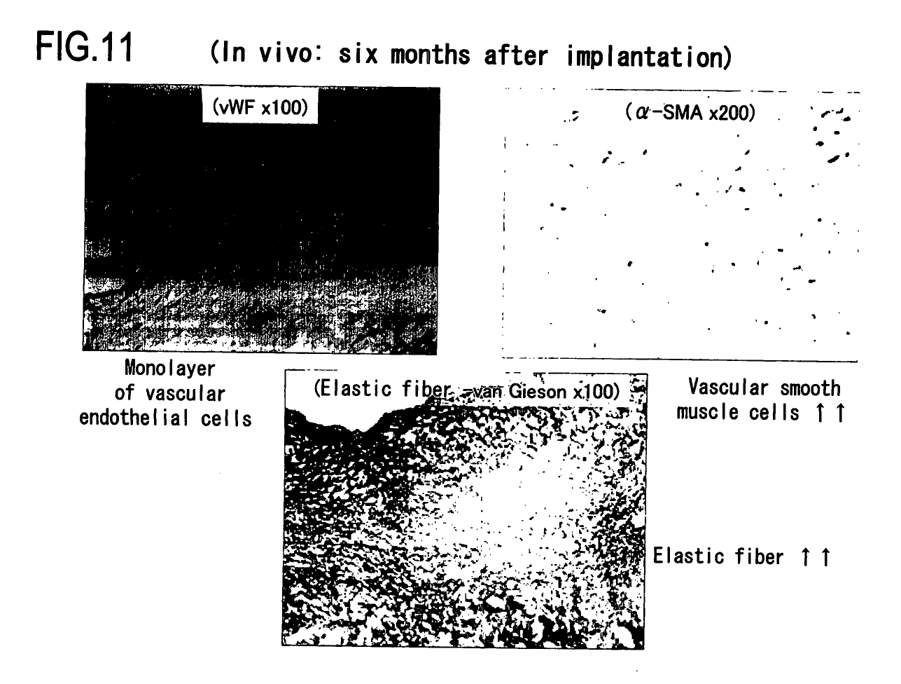

As observed two months after implantation, a monolayer of continuous vascular endothelial cells were observed by Factor VIII immunostaining (FIG. 10). The morphology of the smooth muscle cells was clearly observed as compared to what was observed two months after implantation. α-SMA immunostaining indicated that the smooth muscle cells were aligned on the internal surface and had substantially the same morphology as in normal blood vessels. Elastica van Gieson staining indicated that a larger amount of vascular elastic fiber was observed in an internal layer of a blood vessel than at two months after implantation (FIG. 11).

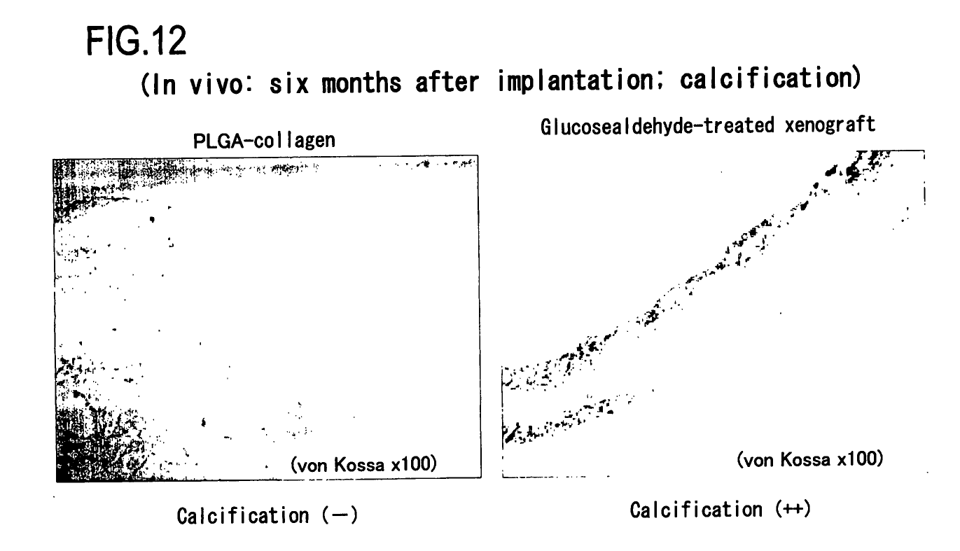

The presence or absence of calcification in blood vessels was studied by von Kossa staining. A positive reaction was not observed in the implanted composite film and blood vessels in its vicinity, i.e., calcification was not observed (FIG. 12).

(Discussion)

Artificial patches (Tissue Engineered Bioprosthetic Patch) which are being developed using tissue engineering techniques aim a structure which constructs extracellular environment approximate to self tissue. Typically, an artificial patch for repairing a blood vessel is used in the cardiovascular surgery field for children and critically needs to be cellularized (possibility of growth). Therefore, it is considered that self cells are cultured on a material having a high level of ability to be absorbed into an organism so as to produce a regenerated blood vessel. In this case, however, self cells have to be collected in advance. There are also a number of additional problems: the isolation of the cells: a technique and a device for culturing the cells; a method for seeding the cells to a structure; and the like.

Recently, there has been a report that progenitor cells are generated in situ. Asahara et al. disproved that blood vessel formation in adults is angiogenesis which is originated from a blood vessel existing in tissue and revealed that there is vasculogenesis in adults, which is a mechanism for creating a new blood vessel from a blood vessel stem cell progenitor cell, as is seen in fetus development (Asahara T., et al. (2000), Stem cell therapy and gene transfer for regeneration, Gene Therapy 7, 451-457: Takahashi T., et al. (1999), Nat. Med. 4, 434-438; Asahara T., et al. (1999), EMBO J., 18, 3964-3972; Isner J., et al. (1999), J. Clin. Invest., 103, 1231-1236; Asahara T., et al. (1997), Science, 275, 964-967).

It has been known for a long time that bone marrow interstitial cells include mesenchymal stem cells which are differentiated into mesenchymal tissues (blood vessel, muscle, fat, bone, cartilage, etc.) (Science, 276, 71-74, 1997). The mesenchymal stem cell has a self-reproducing ability and pluripotency. Orlic et al. has attempted to regenerate a cardiac muscle or a vascular network, which are injured by cardiac muscle infarction, by removing and utilizing bone marrow-derived stem cells so as to ameliorate the function of the heart (Nature, 410, 701-705, 2001; Proc. Natl. Acad. Sci. USA, 98, 10344-10349, 2001; Ann. N.Y. Acad. Sci., 938, 221-229, 2001).

Collagen is a protein most widely present in the animal kingdom, occupying ⅓ or more of the whole animal body by constituting connective tissue of animals, such as skin, tendon, bone, and the like. An animal body is composed of a number of cells. Collagen plays an important role as a matrix between each cell. It is believed that collagen is used in organism only to support the structure of the animal body. However, it has been recently revealed that collagen biologically affects as a intercellular matrix on cells in terms of cell development, differentiation, morphogenesis, and the like (Hiroshi Nagai, Daisaburo Fujimoto, editors, Koragen Taisha to Shikkan [Collagen Metabolism and Diseases], Kodansha (1982); and J. Yang & S. Nandi, Int. Rev. Cytol., 81, 249-286 (1983)). Thus, use of collagen in cell culture may be beneficial. A number of reports have demonstrated that collagen substrates can promote the adhesion, growth, differentiation, and the like of cells to a greater extent than glass substrates and plastic substrates (J. Yang & S. Nandi, Int. Rev. Cytol., 81, 249-286 (1983)). Ehrmannand Gey is the first report to compare the growth of various cells between on collagen and on glass (R. L. Ehrmann & G. O. Gey, Natl. Cancer Inst., 16(6), 1375-1400 (1956)). On 1953, Grobstein reported that collagen substrate has an important role in cell growth and morphogenesis (C. Grobstein, Exp. Zool., 124, 383-388(1953)). It has been reported that the following cells can survive for a longer time on collagen substrates than on plastic or glass culture dishes: corneal endothelial cells (D. Gospodarowicz, G. Greenberg & C. R. Birdwell, Cancer Res., 38, 4155(1978)); mammary gland epithelial cells (M. Wicha, L. A. Liotta, S. Garbisa & W. R. Kidwell, Exp. Cell. Res., 124, 181 (1979)); epidermic cells (J. C. Murray, G. Stingle, H. K. Kleinman, G. R. Martin & S. I. Katz, J. Cell Biol., 80, 197 (1978); hepatocytes (C. A. Sottler, & G. Michalopoulos, Cancer Res., 38, 1539 (1978)), fibroblasts (G. O. Gey, M. Suotelis, M. Foard & F. B. Bang, Exp. Cell Res., 84, 63 (1974)). Therefore, in the present invention, it may be understood that collagen is useful for implantation into the above-described organs or tissues.

In the above-described studies, the survival rate of seeded cells was improved in the case of type I and type IV collagen crosslink treatment. This indicates that type I and type IV collagen have a particularly useful role as extracellular matricies in cell developement, differentiation, morphogenesis, and the like.

In the above-described studies, a support subjected to type I and type IV collagen crosslink treatment was cellularized irrespective of cell seeding. Cells accepted to the structure were no other than self cells. It is inferred that stem cells migrating in organisms are accepted by the structure and differentiated and multiplied at that site using, as a scaffold, a PLGA-collagen composite film subjected to type I and type IV collagen crosslink treatment.

(Summary)

A PLGA-collagen composite film comprising a biodegradable macromolecule is provided. The biodegradable macromolecule is used as a scaffold to achieve reconstruction of blood vessel wall structure without ex vivo cell seeding. The reconstruction of blood vessel wall structure is observed two months after implantation. Even 6 months after implantation, calcification is not observed. It can be expected that the film is useful as an artificial patch for cardiovascular repair, which is cellularized, in the right heart system. Therefore, such an implant has a significant effect which cannot be achieved by conventional techniques.

### Example 2

### Experiment with PGA

In Example 2, PGA was used as a support and type I and type IV collagen were used as biological molecules to prepare an implant. As a result, the effect of the present invention was demonstrated.

(Methods and Results)

Ex Vivo Experiment

<Design of Scaffold>

A sheet of knitted mesh was attached to two sheets of woven mesh (0.2 mm thick for each, a total of 0.6 mm thick). When a resultant patch is implanted into an organism, the knit faces the lumen side thereof while the woven faces the outside thereof. These three sheets of mesh were made of PGA, which is a biodegradable synthetic macromolecule. The resultant structure was subjected to collagen crosslink treatment to obtain a PGA-collagen composite film which was used as a scaffold. Two groups of scaffolds were prepared: A) only type I collagen was used as a crosslinking agent in crosslink treatment; and B) type I and type IV collagen collagen were used. A crosslinking method was conducted as in Example 1.

<Mechanical Strength>

The strength of the PGA-collagen composite film was measured using a tension tester. A weight was loaded on a strip material having a width of 5 mm and a length of 30 mm in a minor axis direction at a rate of 10 mm/min so as to measure the strain at break and the modulus of elasticity thereof (TENSILLON ORIENTEC). As a control, a glutaraldehyde-treated horse pericardium was used for comparison.

<Efficiency of Cell Adhesion>

The cell acceptance ability of the PGA-collagen composite film was determined as follows. The cell adhesion efficiency of vascular endothelial cells (VECs) and vascular smooth muscle cells (VSMCs) labeled with a fluorescent antibody (PKH-26 (SIGMA)) in vitro was compared between a PGA-collagen composite film subjected to crosslink treatment with only type I collagen and a PGA-collagen composite film subjected to crosslink treatment with type I and type IV collagen. The cell adhesion efficiency was determined by the color development area (%) of a fluorescent pigment per visual field of a fluorescence microscope. For both vascular endothelial cells (VECs) and vascular smooth muscle cells (VSMCs), the PGA-collagen composite film subjected to type I and type IV collagen crosslink treatment exhibited a significantly larger color development area of the fluorescent pigment, and cell acceptance was confirmed.

According to the above-described result, the PGA-collagen composite film subjected to type I and type IV collagen crosslink treatment had a strength greater than or equal to that of the conventional glutaraldehyde-treated horse pericardium and had a high level of cell acceptance ability. Next, the PGA-collagen composite film was used to study an in vivo effect of cell seeding before implantation.

<Implantation>

A PGA-collagen composite film (15×10 mm) subjected to type I and type IV collagen crosslink treatment and a film obtained by seeding onto such a composite film self vascular endothelial cells (VECs) and self vascular smooth muscle cells (VSMCs), were prepared. These films were implanted into the pulmonary artery trunk of adult beagle dogs (8 to 10 kg) under partial clamping.

The cells were prepared as follows. A vein was extracted from the anterior surface of a lower limb of an adult beagle dog of the same type. Vascular endothelial cells (VECs) and vascular smooth muscle cells (VSMCs) were isolated from the vein, followed by culture. The vascular endothelial cells and the vascular smooth muscle cells were seeded onto the PGA-collagen composite film at 1.3×10⁶ cells/cm², respectively. After implantation, the film was removed and histologically examined after two weeks, two months, and 6 months.

<In Vivo: Two Weeks After Implantation>

For both the PGA-collagen composite film and the self cell-seeded PGA-collagen composite film, no clear thrombus formation was confirmed using the naked eye. In the case of HE staining, residual PGA was observed and connective tissue was present therebetween. In the PGA-collagen composite film having the seeded self vascular endothelial cells or vascular smooth muscle cells, only seeded fluorescent antibody-labeled vascular endothelial cells were scattered on the internal side of the film. Therefore, it is suggested that most of the cells were detached from the PGA-collagen composite film.

<In Vivo: Two Months After Implantation>

Both the PGA-collagen composite film and the self cell-seeded PGA-collagen composite film had a smooth internal surface observed with the naked eye. HE staining indicated complete absorption of PGA and a tissue structure comparable to normal blood vessels.

The vascular endothelial cells were studied by Factor VIII staining and the vascular smooth muscle cells were studied by α-SMA immunostaining. In both of the films, Factor VIII immunostaining indicated a monolayer of continuous vascular endothelial cells and the α-SMA immunostaining indicated the smooth muscle cells aligned on the internal surface.

Moreover, the vascular elastic fiber was studied by elastica van Gieson staining. In both of the films, elastic fiber was observed in an internal layer of a blood vessel.

<In Vivo: Six Months After Implantation>

As observed two months after implantation, a monolayer of continuous vascular endothelial cells were observed by Factor VIII immunostaining. The morphology of the smooth muscle cells was clearly observed as compared to what was observed two months after implantation. α-SMA immunostaining indicated that the smooth muscle cells were aligned on the internal surface and had substantially the same morphology as in normal blood vessels. Elastica van Gieson staining indicated that a larger amount of vascular elastic fiber was observed in an internal layer of a blood vessel than at two months after implantation.

The presence or absence of calcification in blood vessels was studied by von Kossa staining. A positive reaction was not observed in the implanted composite film and blood vessels in its vicinity, i.e., calcification was not observed.

### Example 3

### Experiment with Sponge-Like PGA

In Example 3, sponge-like PGA was used as a support and type I and type IV collagen were used as biological molecules to prepare an implant. As a result, the effect of the present invention was demonstrated.

(Methods and Results)

Ex Vivo Experiment

<Design of Scaffold>

A sheet of knitted mesh was attached to two sheets of woven mesh (0.2 mm thick for each, a total of 0.6 mm thick). When a resultant patch is implanted into an organism, the knit faces the lumen side thereof while the woven faces the outside thereof. These three sheets of mesh were made of sponge-like PGA, which is a biodegradable synthetic macromolecule. The resultant structure was subjected to collagen crosslink treatment to obtain a sponge-like PGA-collagen composite film which was used as a scaffold. Two groups of scaffolds were prepared: A) only type I collagen was used as a crosslinking agent in crosslink treatment: and B) type I and type IV collagen collagen were used. A crosslinking method was conducted as in Example 1.

<Mechanical Strength>

The strength of the sponge-like PGA-collagen composite film was measured using a tension tester. A weight was loaded on a strip material having a width of 5 mm and a length of 30 mm in a minor axis direction at a rate of 10 mm/min so as to measure the strain at break and the modulus of elasticity thereof (TENSILLON ORIENTEC). As a control, a glutaraldehyde-treated horse pericardium was used for comparison.

<Efficiency of Cell Adhesion>

The cell acceptance ability of the sponge-like PGA-collagen composite film was determined as follows. The cell adhesion efficiency of vascular endothelial cells (VECs) and vascular smooth muscle cells (VSMCs) labeled with a fluorescent antibody (PKH-26 (SIGMA)) in vitro was compared between a sponge-like PGA-collagen composite film subjected to crosslink treatment with only type I collagen and a sponge-like PGA-collagen composite film subjected to crosslink treatment with type I and type IV collagen. The cell adhesion efficiency was determined by the color development area (%) of a fluorescent pigment per visual field of a fluorescence microscope. For both vascular endothelial cells (VECs) and vascular smooth muscle cells (VSMCs), the sponge-like PGA-collagen composite film subjected to type I and type IV collagen crosslink treatment exhibited a significantly larger color development area of the fluorescent pigment, and cell acceptance was confirmed.

### Example 4

### Experiment with Fibronectin

In Example 4, PLGA was used as a support and fibronectin was used as a biological molecule to prepare an implant. As a result, the effect of the present invention was demonstrated.

(Methods and Results)

Ex Vivo Experiment

<Design of Scaffold>

A sheet of knitted mesh was attached to two sheets of woven mesh (0.2 mm thick for each, a total of 0.6 mm thick). When a resultant patch is implanted into an organism, the knit faces the lumen side thereof while the woven faces the outside thereof. These three sheets of mesh were made of a Vicryl polylatin 910 mesh (PLGA (a copolymer having a glycolic acid-to-lactic acid ratio of 90:10)), which is a biodegradable synthetic macromolecule. The resultant structure was subjected to fibronectin crosslink treatment and a treatment for coupling an HGF-fused protein to a collagen-binding domain (FNCBD) to obtain a PLGA-fibronectin composite film which was used as a scaffold. A20 mm-diameter patch was stitched to the pulmonary artery trunk.

<Mechanical Strength>

The strength of the PLGA-fibronectin composite film was measured using a tension tester. A weight was loaded on a strip material having a width of 5 mm and a length of 30 mm in a minor axis direction at a rate of 10 mm/min so as to measure the strain at break and the modulus of elasticity thereof (TENSILLON ORIENTEC). As a control, a glutaraldehyde-treated horse pericardium was used for comparison.

<Efficiency of Cell Adhesion>

The cell acceptance ability of the PLGA-fibronectin composite film was determined as follows. The cell adhesion efficiency of vascular endothelial cells (VECs) and vascular smooth muscle cells (VSMCs) labeled with a fluorescent antibody (PKH-26 (SIGMA)) in vitro was compared between the PLGA-fibronectin composite film and a PLGA-collagen composite film subjected to fibronectin crosslink treatment. The cell adhesion efficiency was determined by the color development area (%) of a fluorescent pigment per visual field of a fluorescence microscope. For both vascular endothelial cells (VECs) and vascular smooth muscle cells (VSMCs), the PLGA-fibronectin composite film subjected to fibronectin crosslink treatment exhibited a significantly larger color development area of the fluorescent pigment, and cell acceptance was confirmed.

### Example 5

### Experiment with HGF-Fused Protein Coupled to the Collagen-Binding Domain (FNCBD) of Fibronectin

In Example 5, PLGA was used as a support and fibronectin was used as a biological molecule to prepare an implant, where an HGF-fused protein is coupled to the collagen binding domain (FNCBb). As a result, the effect of the present invention was demonstrated.

(Methods and Results)

Ex Vivo Experiment

<Design of Scaffold>

A sheet of knitted mesh was attached to two sheets of woven mesh (0.2 mm thick for each, a total of 0.6 mm thick). When a resultant patch is implanted into an organism, the knit faces the lumen side thereof while the woven faces the outside thereof. These three sheets of mesh were made of a Vicryl polylatin 910 mesh (PLGA (a copolymer having a glycolic acid-to-lactic acid ratio of 90:10)), which is a biodegradable synthetic macromolecule. The resultant structure was subjected to fibronectin crosslink treatment and a treatment for coupling an HGF-fused protein to a collagen-binding domain (FNCBD) to obtain a PLGA-fibronectin-HGF composite film which was used as a scaffold. A 20 mm-diameter patch was stitched to the pulmonary artery trunk.

<Mechanical Strength>

The strength of the PLGA-fibronectin-HGF composite film was measured using a tension tester. A weight was loaded on a strip material having a width of 5 mm and a length of 30 mm in a minor axis direction at a rate of 10 mm/mm so as to measure the strain at break and the modulus of elasticity thereof (TENSILLON ORIENTEC). As a control, a glutaraldehyde-treated horse pericardium was used for comparison.

<Efficiency of Cell Adhesion>

The cell acceptance ability of the PLGA-fibronectin-HGF composite film was determined as follows. The cell adhesion efficiency of vascular endothelial cells (VECs) and vascular smooth muscle cells (VSMCs) labeled with a fluorescent antibody (PKH-26 (SIGMA)) in vitro was compared between the PLGA-fibronectin-HGF composite film and a PLGA-collagen-HGF composite film subjected to fibronectin-HGF crosslink treatment. The cell adhesion efficiency was determined by the color development area (%) of a fluorescent pigment per visual field of a fluorescence microscope. For both vascular endothelial cells (VECs) and vascular smooth muscle cells (VSMCs), the PLGA-fibronectin-HGF composite film subjected to fibronectin crosslink treatment exhibited a significantly larger color development area of the fluorescent pigment, and cell acceptance was confirmed.

### Example 6

### Experiment with Blood Vessel-Like Support of PGA

In Example 6, PGA was used as a support and type I and type IV collagen were used as biological molecules to prepare a blood vessel. As a result, the effect of the present invention was demonstrated.

(Methods and Results)

Ex Vivo Experiment

<Design of Scaffold>

A sheet of knitted mesh was attached to two sheets of woven mesh (0.2 mm thick for each, a total of 0.6 mm thick). When a resultant patch is implanted into an organism, the knit faces the lumen side thereof while the woven faces the outside thereof. These three sheets of mesh were made of PGA, which is a biodegradable synthetic macromolecule. The resultant structure was subjected to collagen crosslink treatment to obtain a PGA-collagen composite film which was in turn used as a scaffold to produce an artificial blood vessel. Two groups of scaffolds were prepared: A) only type I collagen was used as a crosslinking agent in crosslink treatment: and B) type I and type IV collagen collagen were used. A crosslinking method was conducted as in Example 1.

<Mechanical Strength>

The strength of the PGA-collagen composite artificial blood vessel was measured using a tension tester. A weight was loaded on a strip material having a width of 5 mm and a length of 30 mm in a minor axis direction at a rate of 10 mm/mm so as to measure the strain at break and the modulus of elasticity thereof (TENSILLON ORIENTEC). As a control, a woven Dacron was used for comparison.

<Efficiency of Cell Adhesion>

The cell acceptance ability of the PGA-collagen composite artificial blood vessel was determined as follows. The cell adhesion efficiency of vascular endothelial cells (VECs) and vascular smooth muscle cells (VSMCs) labeled with a fluorescent antibody (PKH-26 (SIGMA)) in vitro was compared between a PGA-collagen composite artificial blood vessel subjected to crosslink treatment with only type I collagen and a PGA-collagen composite artificial blood vessel subjected to crosslink treatment with type I and type IV collagen. The cell adhesion efficiency was determined by the color development area (%) of a fluorescent pigment per visual field of a fluorescence microscope. For both vascular endothelial cells (VECs) and vascular smooth muscle cells (VSMCs), the PGA-collagen composite artificial blood vessel subjected to type I and type IV collagen crosslink treatment exhibited a significantly larger color development area of the fluorescent pigment, and cell acceptance was confirmed.

According to the above-described result, the PGA-collagen composite artificial blood vessel subjected to type I and type IV collagen crosslink treatment had a strength greater than or equal to that of the conventional glutaraldehyde-treated horse pericardium and had a high level of cell acceptance ability. The PGA-collagen composite artificial blood vessel was used to study an in vivo effect of cell seeding before implantation.

### Example 7

### Experiment with HGF-Fused Protein Coupled to the Collagen-Binding Domain (FNCBD) of Fibronectin being Applied to Heart

In Example 7, PLGA was used as a support and fibronectin was used as a biological molecule to prepare an implant, where an HGF-fused protein is coupled to the collagen binding domain (FNCBD). As a result, the effect of the present invention was demonstrated.

(Methods and Results)

Ex Vivo Experiment

<Design of Scaffold>

A sheet of knitted mesh was attached to two sheets of woven mesh (0.2 mm thick for each, a total of 0.6 mm thick). When a resultant patch is implanted into an organism, the knit faces the lumen side thereof while the woven faces the outside thereof. These three sheets of mesh were made of a Vicryl polylatin 910 mesh (PLGA (a copolymer having a glycolic acid-to-lactic acid ratio of 90:10)), which is a biodegradable synthetic macromolecule. The resultant structure was subjected to fibronectin crosslink treatment and a treatment for coupling an HGF-fused protein to a collagen-binding domain (FNCBD) to obtain a PLGA-fibronectin-HGF composite film which was used as a scaffold. Myocardial infarction was created in adult beagle dogs (8 to 10 kg). A 20 mm-diameter patch was stitched to the myocardial infarction site.

<Mechanical Strength>

The strength of the PLGA-fibronectin-HGF composite film was measured using a tension tester. A weight was loaded on a strip material having a width of 5 mm and a length of 30 mm in a minor axis direction at a rate of 10 mm/min so as to measure the strain at break and the modulus of elasticity thereof (TENSILLON ORIENTEC). As a control, a glutaraldehyde-treated horse pericardium was used for comparison.

<Efficiency of Cell Adhesion>

The cell acceptance ability of the S PLGA-fibronectin-HGF composite film was determined as follows. The cell adhesion efficiency of vascular endothelial cells (VECs) and myocardial cells labeled with a fluorescent antibody (PKH-26 (SIGMA)) in vitro was compared between the PLGA-fibronectin-HGF composite film and a PLGA-collagen-HGF composite film subjected to collagen-HGF crosslink treatment. The cell adhesion efficiency was determined by the color development area (%) of a fluorescent pigment per visual field of a fluorescence microscope. For both vascular endothelial cells (VECs) and myocardial cells, the PLGA-fibronectin-HGF composite film subjected to fibronectin crosslink treatment exhibited a significantly larger color development area of the fluorescent pigment, and cell acceptance was confirmed.

When the PLGA-fibronectin-HGF composite film was implanted to a myocardial infarction site, it was confirmed that the cardiac muscle site was occupied by regenerated cardiac muscle and new blood vessels were formed. Cardiac muscle cells thereof have a phenotype similar to that of Lin-, c-kit+bone marrow mesenchymal cells and were confirmed to perform organ regeneration and tissue formation within self tissue.

### Example 8

### Experiment with Laminin

In Example 8, PLGA was used as a support and laminin was used as a biological molecule to prepare an implant. As a result, the effect of the present invention was demonstrated.

(Methods and Results)

Ex Vivo Experiment

<Design of Scaffold>

A sheet of knitted mesh was attached to two sheets of woven mesh (0.2 mm thick for each, a total of 0.6 mm thick). When a resultant patch is implanted into an organism, the knit faces the lumen side thereof while the woven faces the outside thereof. These three sheets of mesh were made of a Vicryl polylatin 910 mesh (PLGA (a copolymer having a glycolic acid-to-lactic acid ratio of 90:10)), which is a biodegradable synthetic macromolecule. The resultant structure was subjected to laminin crosslink treatment to obtain a PLGA-laminin composite film which was used as a scaffold. A 20 mm-diameter patch was stitched to the pulmonary artery trunk.

<Mechanical Strength>

The strength of the PLGA-laminin composite film was measured using a tension tester. A weight was loaded on a strip material having a width of 5 mm and a length of 30 mm in a minor axis direction at a rate of 10 mm/min so as to measure the strain at break and the modulus of elasticity thereof (TENSILLON ORIENTEC). As a control, a glutaraldehyde-treated horse pericardium was used for comparison.

<Efficiency of Cell Adhesion>

The cell acceptance ability of the PLGA-laminin composite film was determined as follows. The cell adhesion efficiency of vascular endothelial cells (VECs) and vascular smooth muscle cells (VSMCs) labeled with a fluorescent antibody (PKH-26 (SIGMA)) in vitro was compared between the PLGA-laminin composite film and a PLGA-collagen composite film subjected to laminin crosslink treatment. The cell adhesion efficiency was determined by the color development area (%) of a fluorescent pigment per visual field of a fluorescence microscope. For both vascular endothelial cells (VECs) and vascular smooth muscle cells (VSMCs), the PLGA-laminin composite film subjected to laminin crosslink treatment exhibited a significantly larger color development area of the fluorescent pigment, and cell acceptance was confirmed.

### Example 9

### Production of Support: Production of Knit and Woven

Wovens were produced as a mesh of poly(glycolic acid) and a mesh of poly(L-lactic acid) using a method known in the art. The procedure is described below. As a thread, a multifilament (64 f (filament) and 240 d (denier)) was used. A plain weave was used (warp: about 64 threads/inch; weft: about 40 to 47.5 threads/inch).

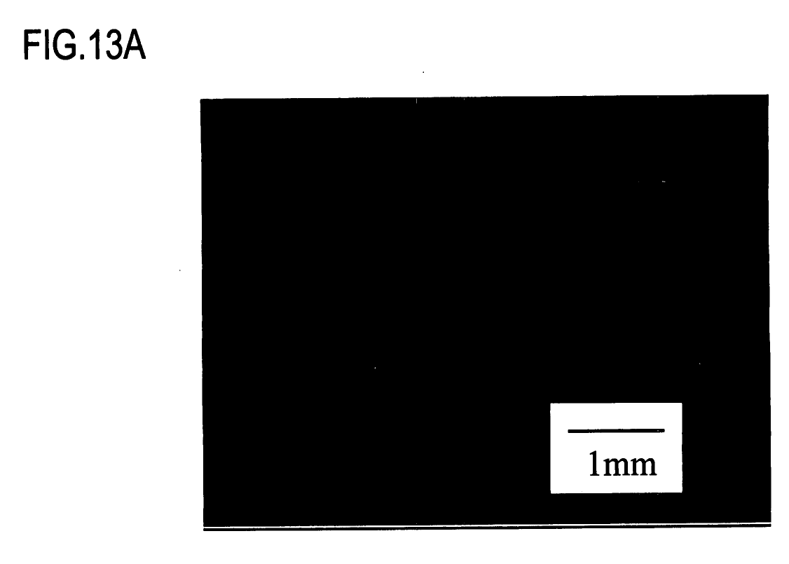

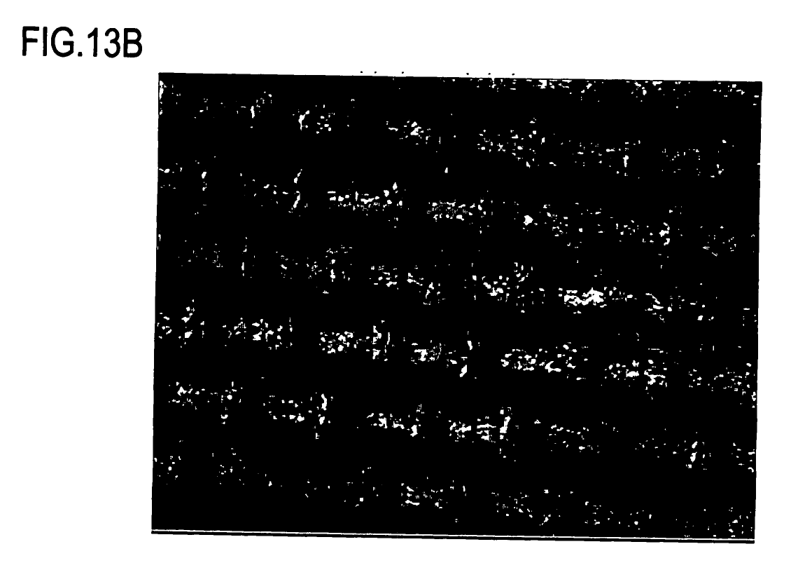

The prepared poly(glycolic acid) and poly(L-lactic acid) mesh are shown in FIGS. 13A and 13B.

A knit made of poly(glycolic acid) was produced by a known method in the art. The procedure is described below. As a thread, a multifilament (about 68 d: about 30 f) was used. The knit was knitted by the following method.

Combination:

No. 1: AL1, AL2, AL3

No. 2: AL1, AL2, AL3 (more L2 feeds than No. 1)

No. 3: BL1, AL2, AL3 (used in cell adhesion experiments)

No. 4: BL1, AL2, AL3 (more L3 needle per inch than No. 3)

No. 5: BL1, AL2, AL3 (more L2 feeds than No. 4)

No. 6: BL1, AL2, AL3 (more L3 needle per inch than No. 2)

No. 7: BL1, AL2, AL3 (more L2 feeds than No. 6)

No. 8: BL1, BL2, AL3

TABLE 1
(Examples of Knitting Fashions)

| | L1 | L2 | L3 |
|---|---|---|---|
| A | 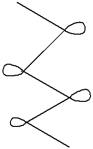 | 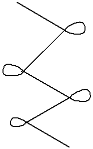 | 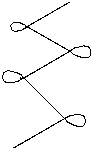 |
| B | 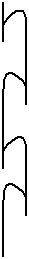 | 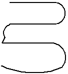 | |

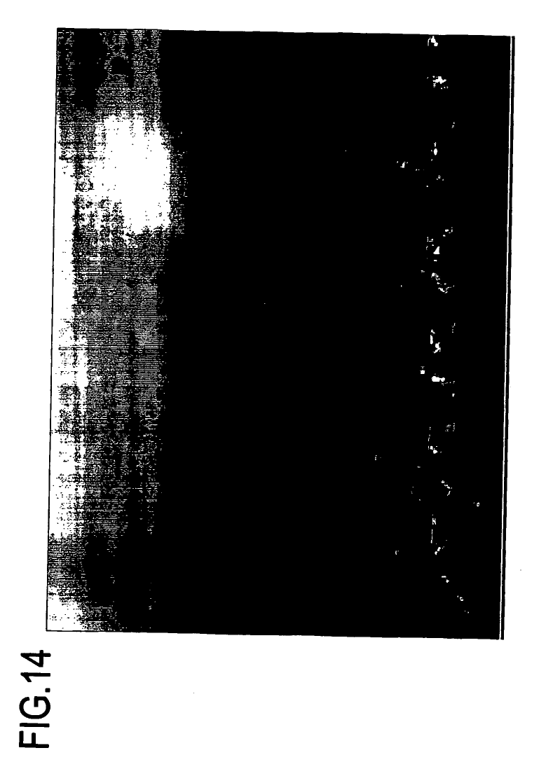

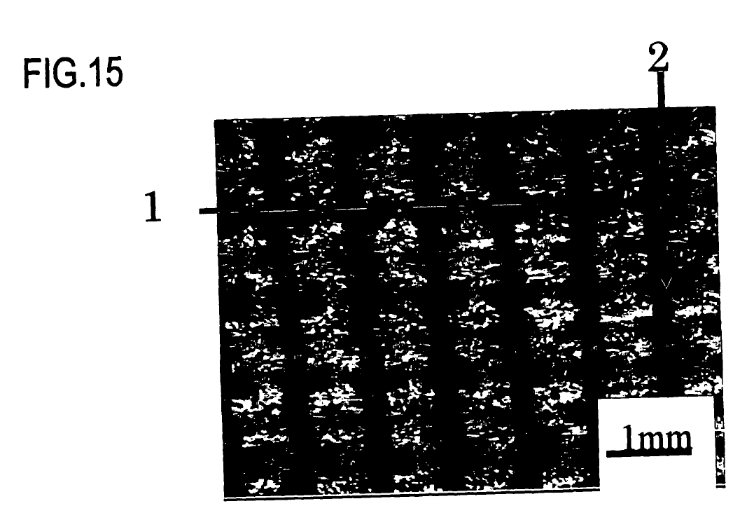

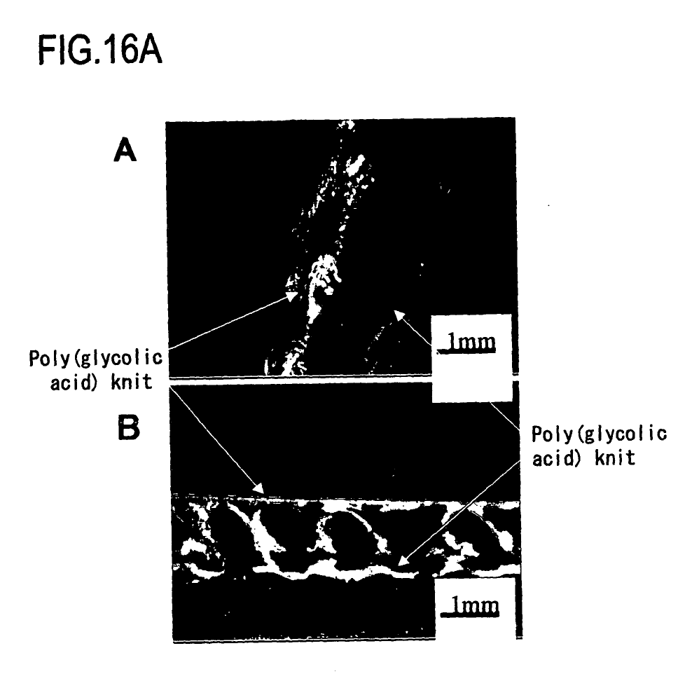

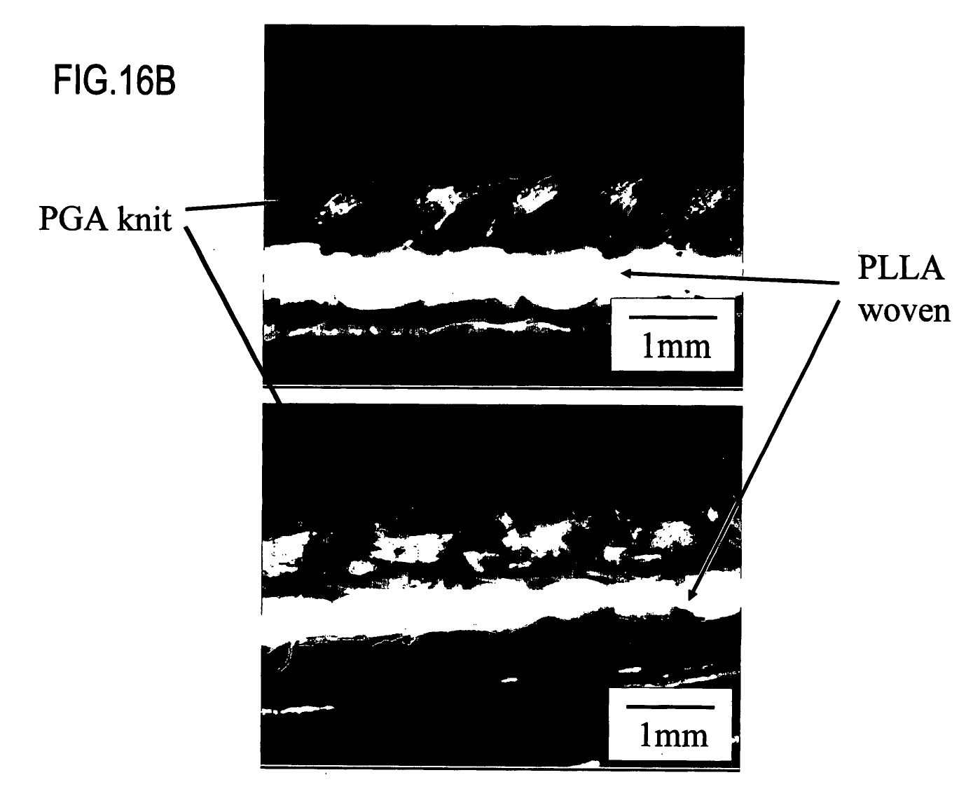

The produced poly(glycolic acid) knit is shown in FIGS. 14 and 15. FIGS. 16A and 16B show a composite material of a poly(glycolic acid) knit and a poly(glycolic acid) woven and a composite of a poly(glycolic acid) knit and a poly(L-lactic acid) woven, respectively.

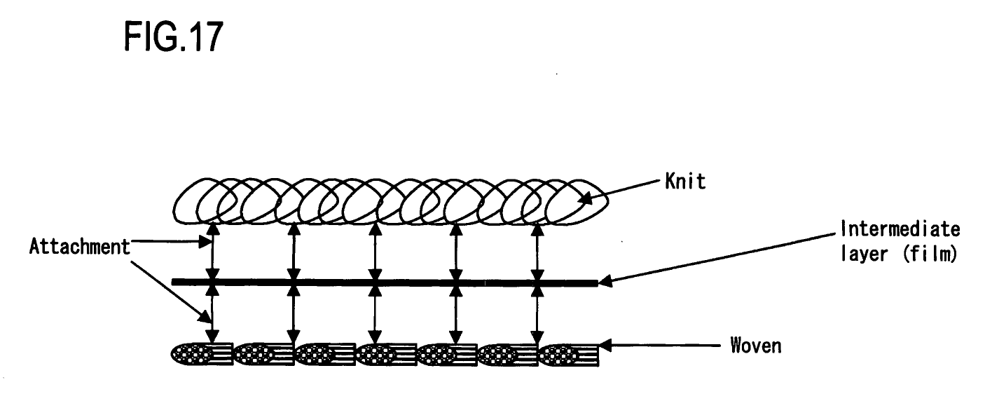

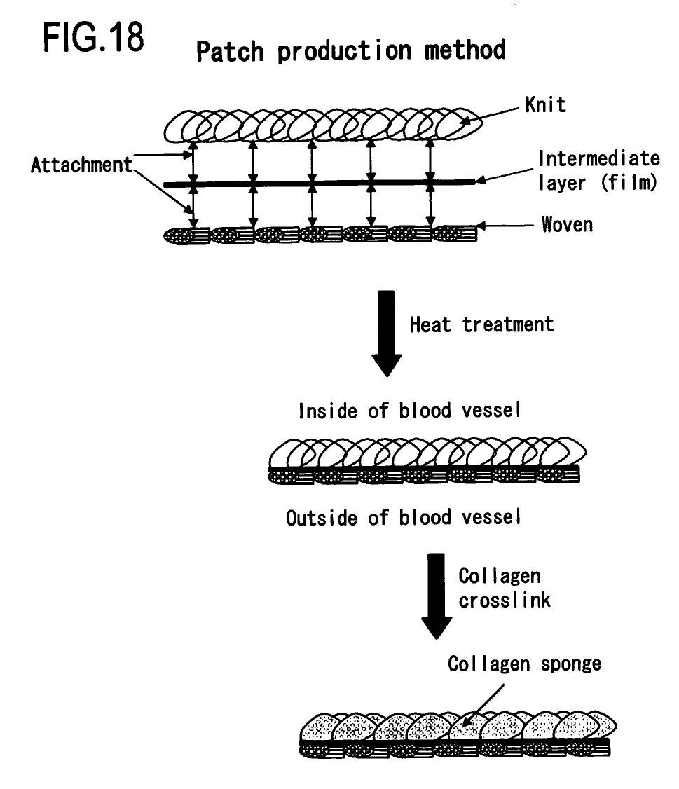

Next, the knit and the woven were attached together via a film as an intermediate layer. An attaching method is schematically shown in FIGS. 17 and 18 (detailed figure).

The film was produced by casting a material (poly(lactic acid) or caprolactam) on a glass plate, followed by air freezing.

Next, the woven was laid down, the poly(lactic acid) film was laid on the woven, and the knit of poly(glycolic acid) was laid on the film. Thereafter, heat treatment was conducted (between 80° C. to 140° C.) to attach these layers together.

The resultant support can be used as a graft.

### Example 10

### Attachment of Biological Molecule

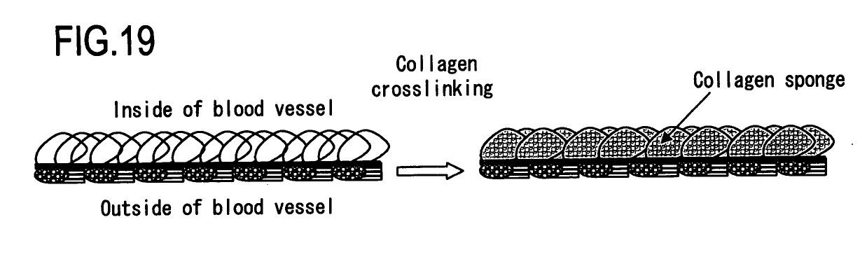

As biological molecules, collagen (type I and type IV) and laminin were attached to the support produced in Example 9. The resultant support is schematically shown in FIGS. 18 and 19.

Thereafter, collagen and laminin were subjected to crosslink treatment. A crosslinking method is described in Example 1.

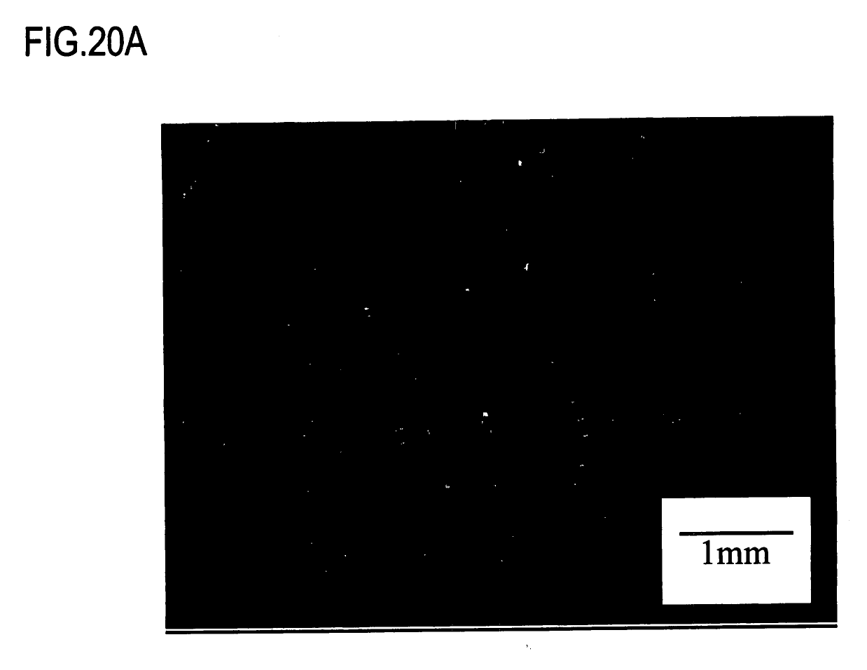

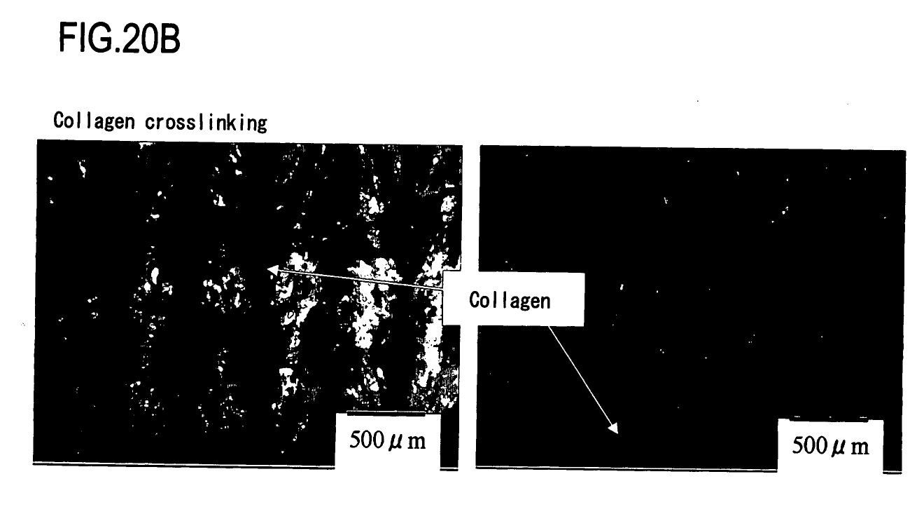

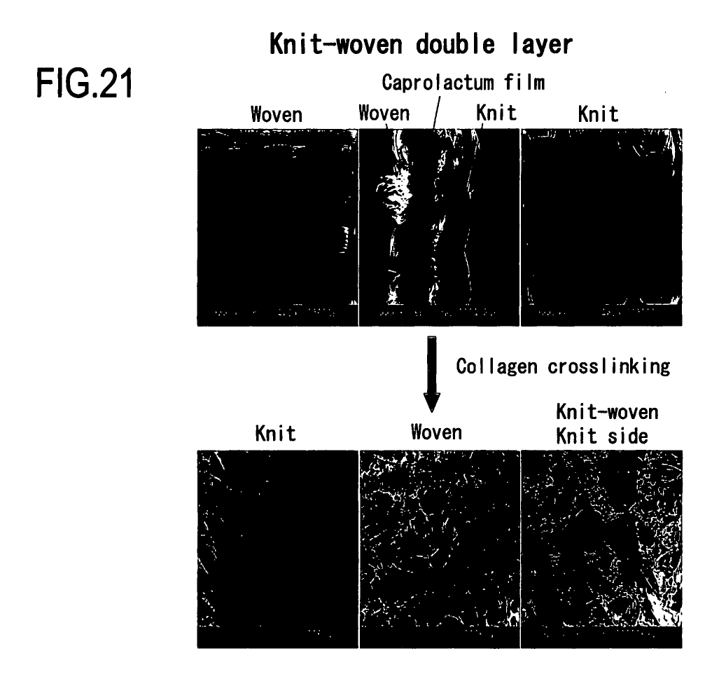

After collagen crosslink treatment, collagen was attached to the support as shown in FIGS. 20A and 20B (a poly(glycolic acid) woven and a poly(L-lactic acid) woven, respectively). A difference in collagen crosslink was examined between a woven and a knit as shown in FIG. 21.

In this manner, the following various biological molecule supports were produced.

1. PGA knit No. 3-PLA woven weft

2. PGA knit No. 3-PLA woven warp

3. PLA woven 47.5 weft (Comparative Example)

4. PLA woven 47.5 warp (Comparative Example)

5. PGA knit No. 3 weft

6. PGA knit No. 3 warp

In experiments below, a Hamshield artificial blood vessel (Hamshiled Platinum™ Woven Vascular Grafts, Boston Scientific, MA, USA) and a Vascutek artificial blood vessel (Gelseal™, Terumo, Japan) were used as controls.

### Example 11

### Function of Biological Molecule Support

Next, the tensile strength, modulus of elasticity, and strain of the collagen support produced in Example 10 were determined by a tension test as described below.

In Example 11, a tension tester (TENSILLON ORIENTEC) was used to measure the strength. Specifically, a weight was loaded on a strip material having a width of 5 mm and a length of 30 mm in a minor axis direction at a rate of 10 mm/min so as to measure the strain at break and the modulus of elasticity thereof.

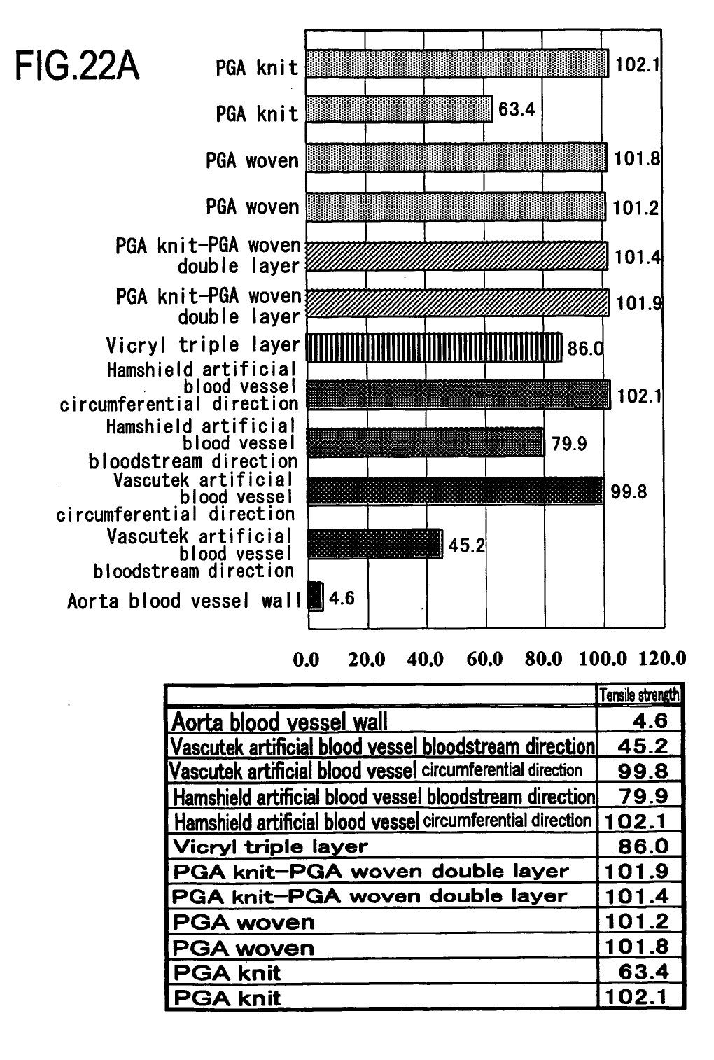

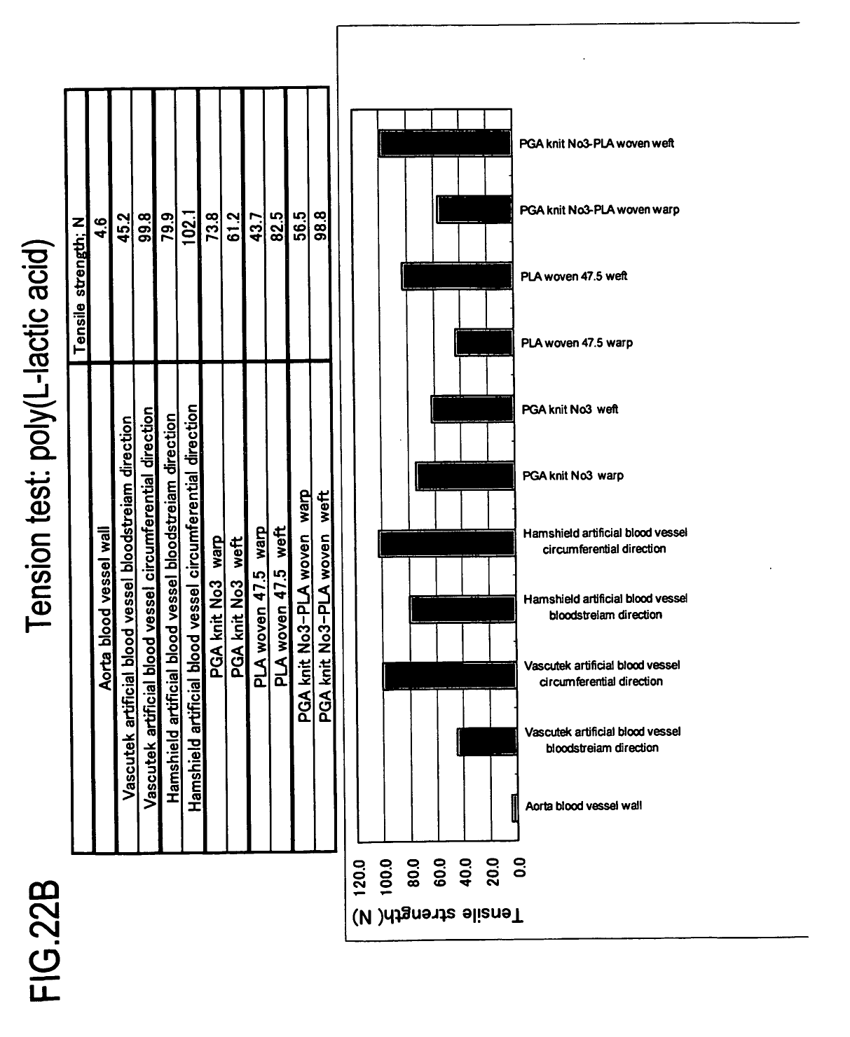

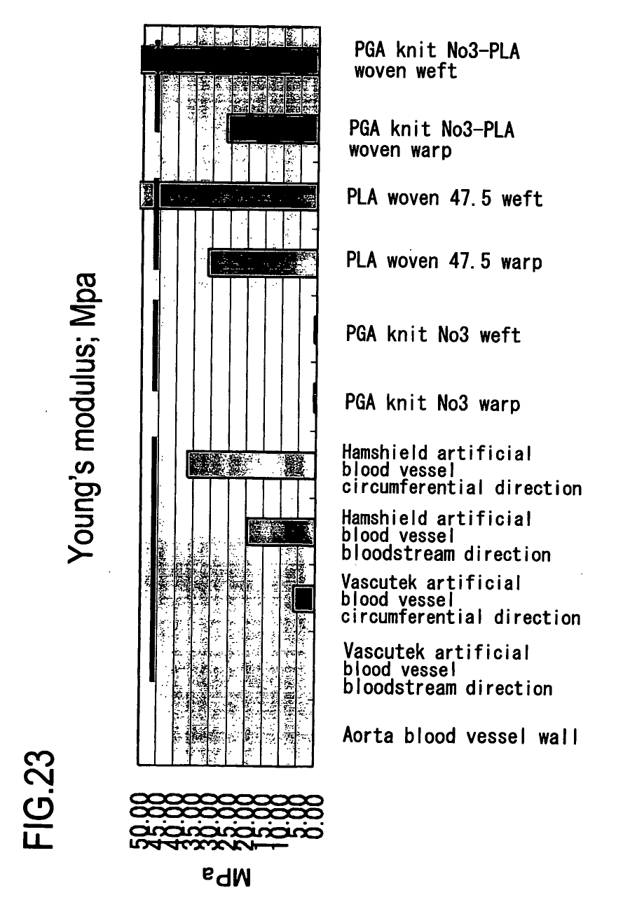

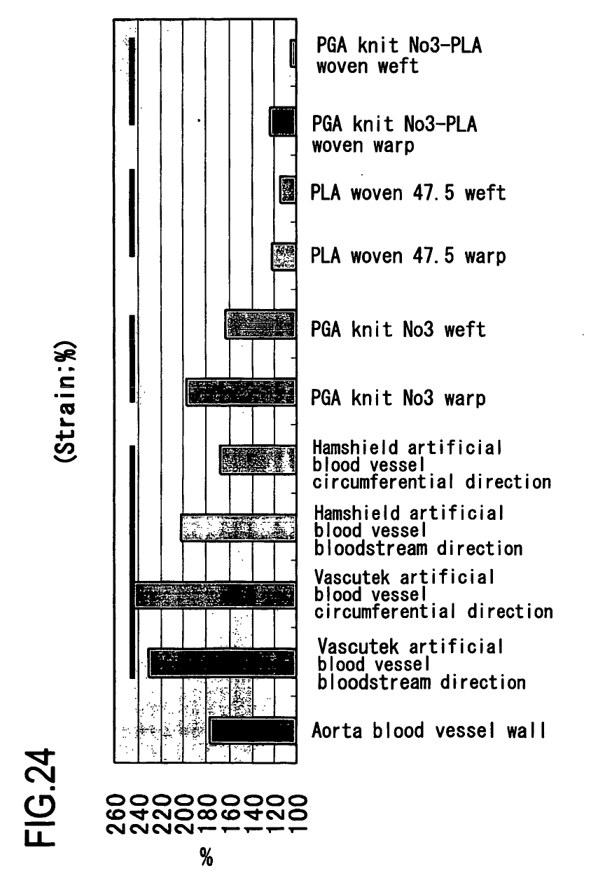

The results are shown in FIGS. 22 to 24. These figures show the tensile strength, the modulus of elasticity, and the strain, respectively. FIG. 22A shows the result of a combination of a poly(glycolic acid) knit and a poly(glycolic acid) woven and FIG. 22B shows the result of a combination of a poly(glycolic acid) knit and a poly(L-lactic acid) woven.

The results reveal that the support of the present invention has a strength and modulus of elasticity greater than or equal to an aorta blood vessel wall and a commercially available artificial blood vessel wall as controls. It was also revealed that the strain fell within a tolerable range.

Next, the water leakage rate and the air permeability of the support were studied in accordance with the following protocol.

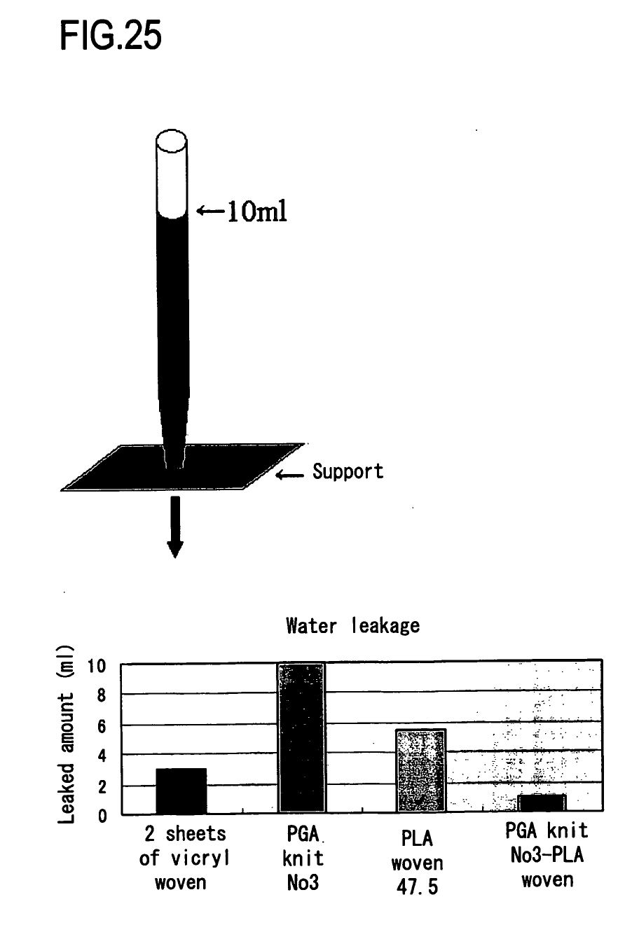

The water leakage rate was determined by holding the support horizontally, adding 10 ml of water thereon in a dropwise manner, and measuring the amount of leaking water for 60 sec. The result is shown in FIG. 25. The results show that the support of the present invention substantially prevents leakage of blood or the like.

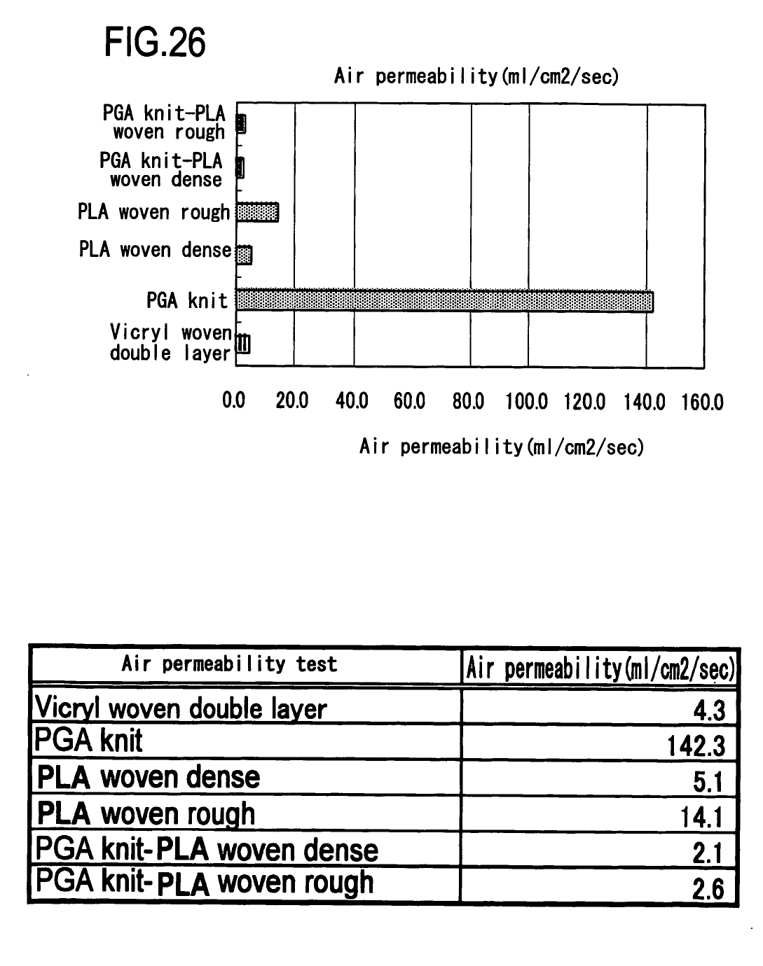

Next, the air permeability of the support of the present invention and another support was determined. In Example 11, JIS-H-1096A protocol was used. Specifically, a test piece was attached to a Frazil-type Air Permeability Tester. A pressurize resister was used to adjust pressure to 125 Pa while measuring the pressure by an inclined-type barometer. The amount of passing air (ml/cm²/sec) was measured to determine an air permeability. The result is shown in FIG. 26. A double-layer Vicryl woven was used as a control, which had been known to prevent blood leakage when it was implanted in a dog. The above-described prepared double-layer mesh had substantially the same air permeability as that of the control, i.e., 2.0 ml/cm²/sec or less. Therefore, the air permeability test also demonstrated that the support of the present invention prevents blood leakage.

### Example 12

### Cellular Adhesiveness of Biological Molecule Support

Next, the cellular adhesiveness of a biological molecule support of the present invention was determined. This test was conducted using the support produced in Example 10. 1×10⁵ vascular endothelial cells were seeded to each support (1×1 cm²). After 15 hours of culture, MTT assay was conducted to measure absorbance at 595 nm. A procedure for MTT assay is described as follows. The support was washed with culture medium. The cells were cultured in medium supplemented with a 1/10 volume of MTT solution at 37° C. for 1 hour. After culturing, the support was washed with PBS. Acid isopropanol was added to the solution, followed by shaking for 10 min. The absorbance of the solution was measured at 595 nm using a micro plate reader to determine a standard for MTT.

MTT is an assessment method for cellular activity based on the fact that a tetrazolium salt is reduced to formazan by mitochondrial dehydrogenase within cells. The amount of formazan produced satisfactorily corresponds to the number of cells. Also, formazan has an absorption characteristic with respect to a specific wavelength. Therefore, the number of surviving cells can be easily determined by measuring the absorbance of a sample. By measuring the metabolism activity of intracellular mitochondria, cell death can be detected at a relatively early stage.

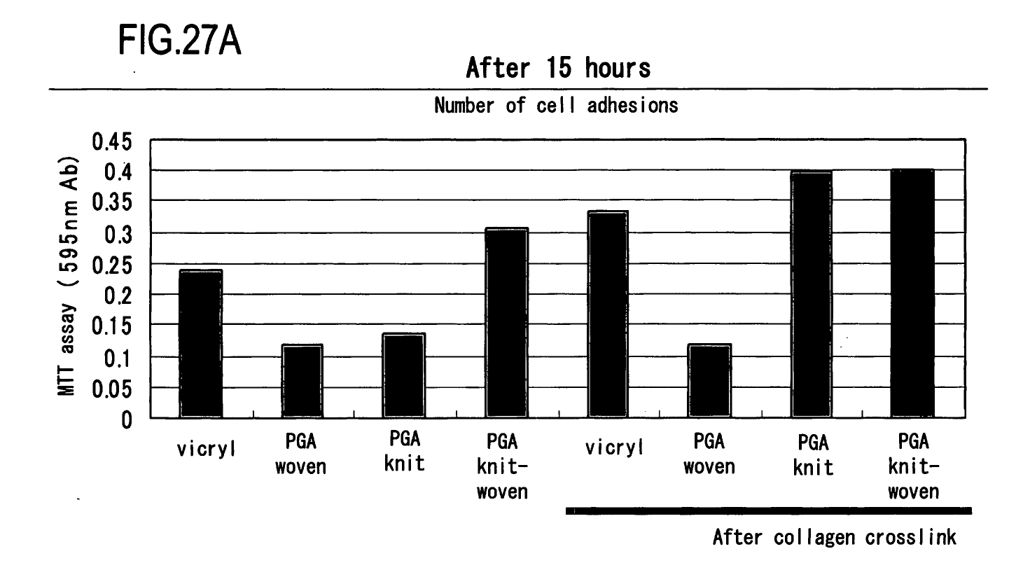

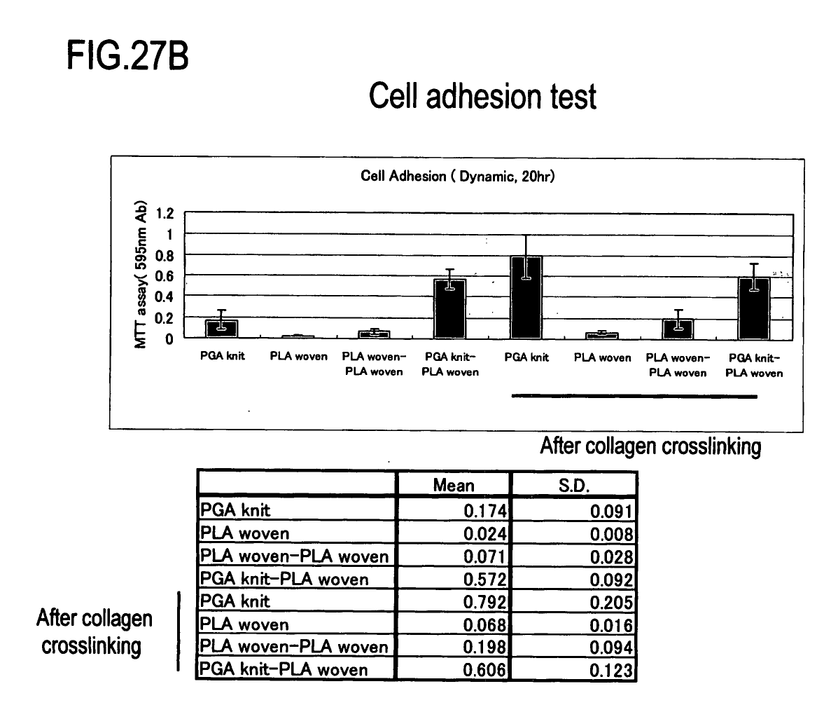

The result is shown in FIG. 27, indicating a state after 15 hours. FIG. 27A shows a combination of a poly(glycolic acid) knit and a poly(glycolic acid) woven and FIG. 27B shows a combination of a poly(glycolic acid) knit and a poly(L-lactic acid) woven. Accordingly, collagen crosslinking improved the cellular adhesiveness to a greater extent. This improvement was also found for other extracellular matrices (e.g., laminin, fibronectin, etc.). It was revealed that wovens have a higher level of cellular adhesiveness than knits after collagen crosslink treatment.

It was also revealed that the support of the present invention has no problem in cellular adhesiveness.

### Example 13

### Study on Attachment Conditions

Next, conditions for attachment in a support of the present invention were studied.

A caprolactam film was provided as an intermediate layer between a poly(L-lactic acid) woven and a poly(glycolic acid) woven to study various attachment conditions. The caprolactam film was produced by casting 5% caprolactam/dioxane solution 300 μm thick on a glass plate, followed by air drying. The production method is shown in FIG. 28.

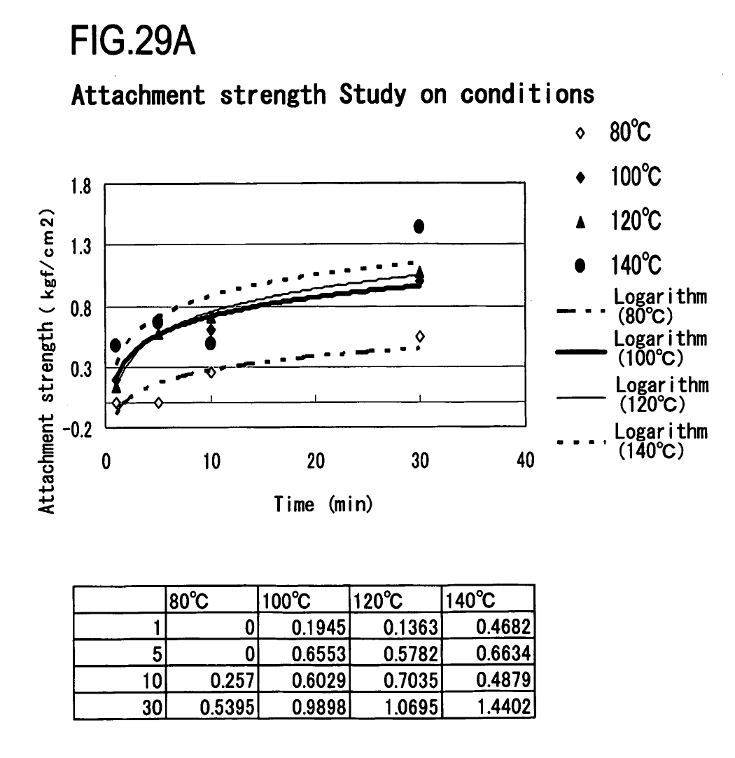

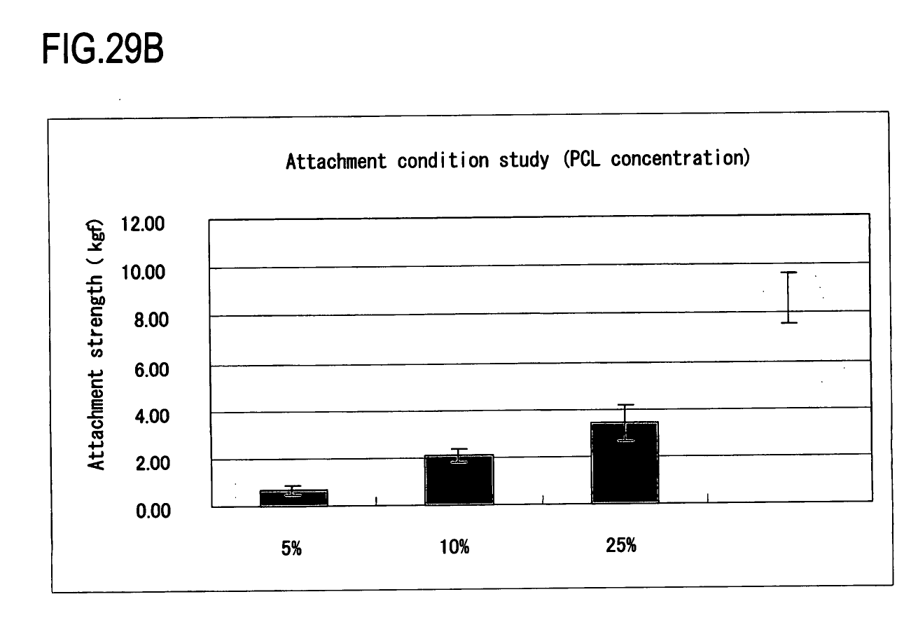

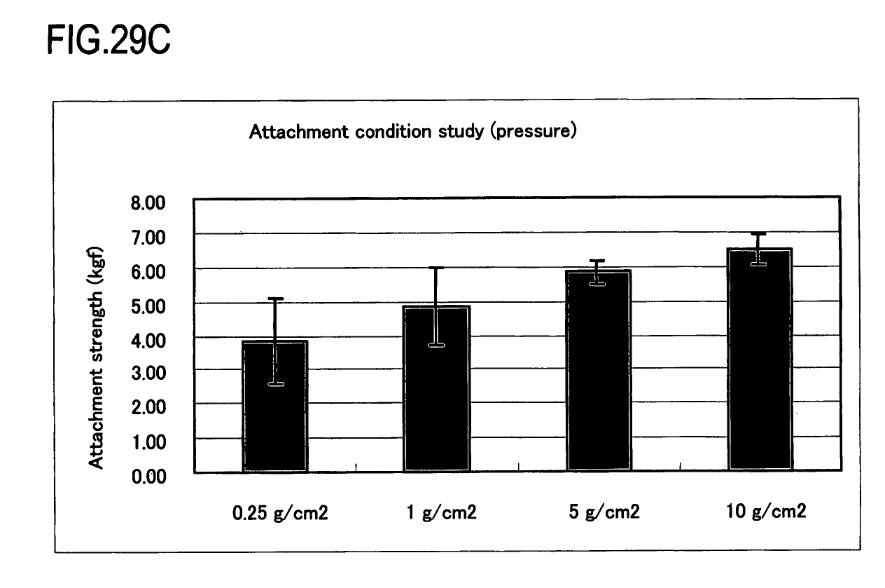

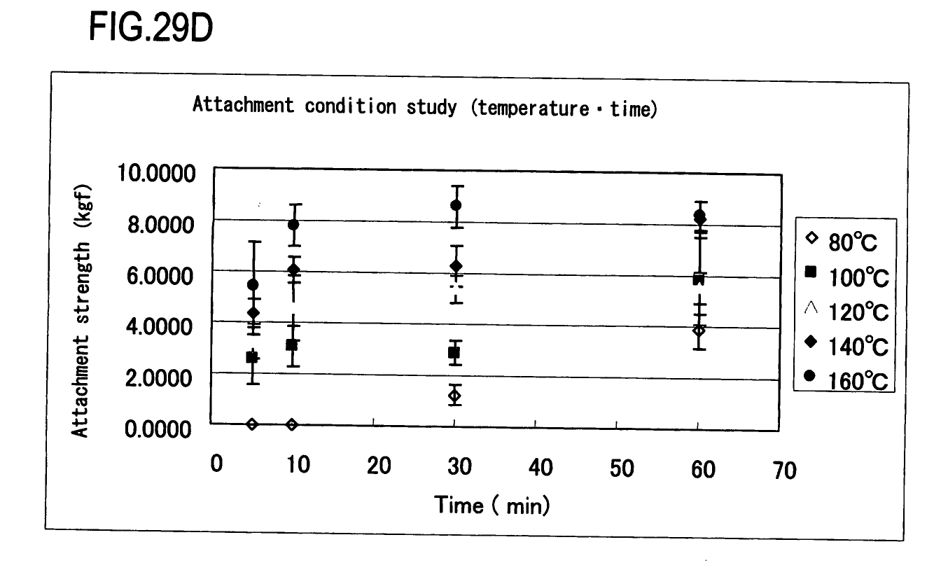

Next, the attachment strength was determined in substantially the same manner as described in Example 11. The result is shown in FIG. 29A. Accordingly, the strength was significantly improved in the range from about 80° C. to 140° C., i.e., a temperature higher than the melting point of the intermediate layer and lower than the melting point of the first layer and the second layer. Therefore, a temperature around that temperature is preferably used for production of the support of the present invention. In this case, it was revealed that the treatment is conducted for at least 10 min.

Various parameters for attachment conditions were studied.

Attachment conditions were examined in terms of the amount of caprolactone, pressure exerted on the support from the top during attachment, temperature, and time. Attachment strength was measured as in the above-described examples.

The result of concentration dependency of PCL is shown in the table below and FIG. 29B.

Attachment condition study
(PCL concentration)
140° C. 30 min 0.7 g/cm²

| | Attachment | Attachment |
|---|---|---|
| PCL | Strangth | Strangth |
| concentration | Mean | S.D. |
| 5% | 0.66 | 0.21 |
| 10% | 2.12 | 0.28 |
| 25% | 3.40 | 0.79 |
| 25% × 2 | 8.57 | 1.04 |

The result of pressure dependency of PCL is shown in the table below and FIG. 29C.

Attachment conditions study (pressure)
140° C. 30 min 15% PCL

| | Attachment | Attachment |
|---|---|---|
| | Strength | Strength |
| Pressure | Mean | S.D. |
| 0.25 g/cm² | 3.84 | 1.27 |
| 1 g/cm² | 4.85 | 1.13 |
| 5 g/cm² | 5.83 | 0.36 |
| 10 g/cm² | 6.51 | 0.45 |

Next, it was found that attachment strength was increased with an increase in the amount of caprolactone used for attachment. Also, attachment strength was increased with an increase in pressure exerted on the support from the top within the range examined herein.

<Temperature Condition>

Next, attachment strength was examined in terms of temperature and time. In Example 27, PGA and PLGA were attached together with caprolactone.

Attachment strength was increased with an increase in temperature and time within the ranges examined herein.

The results are shown in FIG. 29D.

Attachment condition study (Temperature, time)
5 g/cm² 15% PCL
Attachment strength (kgf)

| | 5 min | 10 min | 30 min | 60 min |
|---|---|---|---|---|
| Mean | | | | |
| 80° C. | 0.0000 | 0.0000 | 1.2376 | 3.8297 |
| 100° C. | 2.5477 | 3.0817 | 2.8792 | 5.8737 |
| 120° C. | 3.2445 | 4.5950 | 5.4031 | 5.5375 |
| 140° C. | 4.3790 | 6.1050 | 6.2982 | 8.2065 |
| 160° C. | 5.4477 | 7.8010 | 8.6098 | 8.3670 |
| S.D. | | | | |
| 80° C. | 0.0000 | 0.0000 | 0.4028 | 0.7059 |
| 100° C. | 0.9618 | 0.7964 | 0.4700 | 1.8122 |
| 120° C. | 0.7004 | 1.2751 | 0.5268 | 0.6335 |
| 140° C. | 0.5688 | 0.4987 | 0.8073 | 0.7068 |
| 160° C. | 1.6772 | 0.8173 | 0.8369 | 0.5566 |

As a result, it was found that attachment strength can be increased with increases in the amount of caprolactone, pressure exerted from the top, temperature, and time within the ranges examined herein, though examination of other conditions (rigidity, thickness, etc.) may be required.

### Example 14

### Strength Deterioration Test

Next, a strength deterioration test was conducted in vitro.

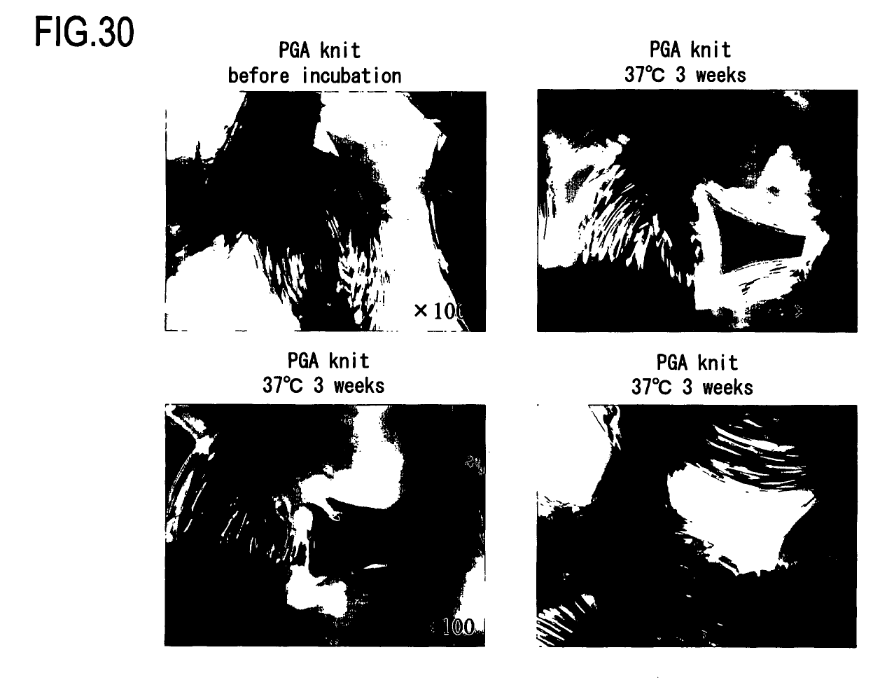

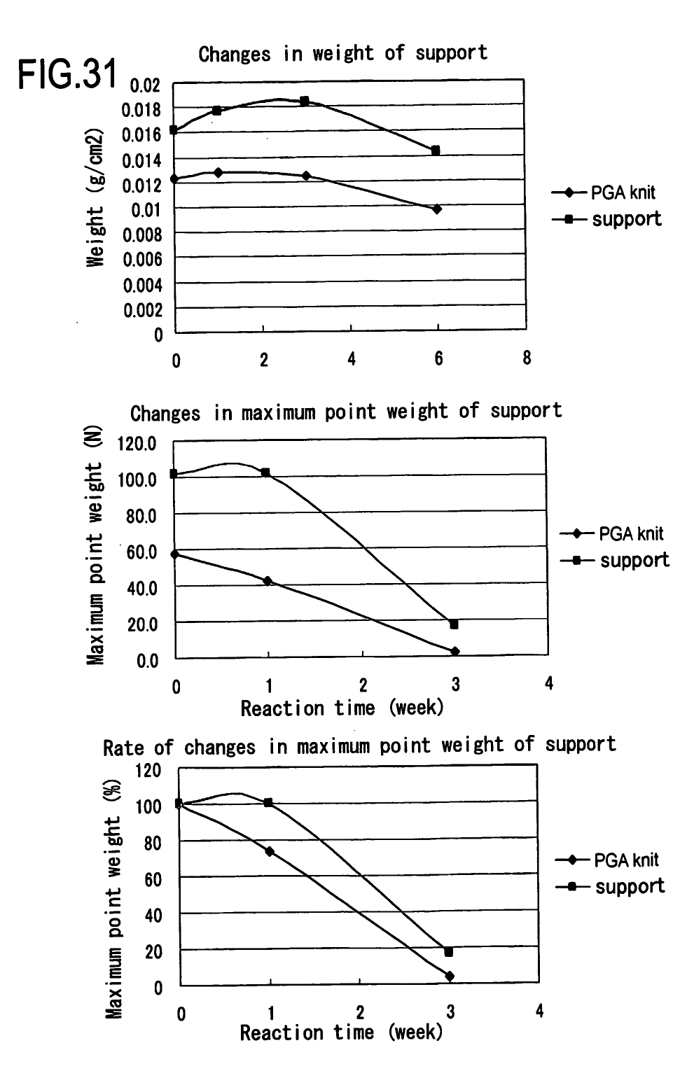

In order to predict the strength deterioration of PGA in organisms for an implantation period, a degradation test was conducted. The procedure is described as follows. A support of the present invention and a Dexon mesh (control) were placed in PBS at 37° C., and the outer appearance, weight, and tensile strength thereof were observed after 1, 3 and 6 weeks. The changes are shown in FIG. 30. Values are plotted on a graph as shown in FIG. 31.

As can be seen from FIG. 31, the strength was substantially completely impaired after 3 weeks. It is considered that the support is degraded in organisms in substantially the same manner as in the assay. The support is considered to lose its strength 3 weeks after implantation.

### Example 15

### Other Extracellular Matrices

Next, it was determined whether or not other extracellular matrices can be used to produce the same support as when collagen is used.

As extracellular matrices, type I collagen, type IV collagen and laminin were used. As cells, vascular endothelial cells and vascular smooth muscle cells were used. As an assay, the above-described MTT assay was used.

In Example 15, the support produced had a sufficient strength (a support of the present invention: 101.4 N, an aorta wall of an adult dog: 5.4 N, conventional artificial blood vessel (Hamshield and Gelseal): 101.4 N).

An air permeability test was conducted as described above. As a result, the support of the present invention had an air permeability of 2.1 ml/cm²/sec, the woven had an air permeability of 5.1 ml/cm²/sec, and the knit had an air permeability of 142.3 ml/cm²/sec.

A cellular adhesiveness test was conducted as described in the above-described Examples. As a result, the following values were obtained: the woven, 0.116±0.005; the knit (with a collagen sponge), 0.398±0.008; and the support of the present invention, 0.402±0.035. Therefore, use of a collagen sponge significantly improved cellular adhesiveness.

The cellular adhesiveness was the following. In the case of type I collagen, vascular endothelial cell: 0.145±0.053/vascular smooth muscle cell: 0.286±0.032. In the case of type IV collagen, vascular endothelial cell: 0.159±0.056/vascular smooth muscle cell: 0.252±0.016. In the case of laminin, vascular endothelial cell: 0.146±0.017/vascular smooth muscle cell: 0.251±0.014. It was revealed that most extracellular matrices were similarly effective.

Therefore, the support of the present invention has a sufficient strength which allows it to be used as a repair patch for regeneration of self tissue, such as cardiovascular tissue and other tissues; less blood leakage; and a high level of cell acceptance ability. Thus, the support of the present invention can be used as a repair support for cardiovascular tissue and other tissues in clinical applications.

### Example 16

### In Vivo Test

The support of the present invention produced in Example 10 (with collagen and without collagen; 15 mm×10 mm) was implanted into the pulmonary artery trunk of adult beagle dogs (8 to 12 kg). The part was extracted 2 weeks, 2 months, or 6 months after implantation and histologically examined.

<In Vivo: Two Weeks After Implantation>

No clear thrombus formation was observed in the implanted support with the naked eye. In the case of HE staining, support residue was observed and connective tissue was present therebetween.

<In Vivo: Two Months After Implantation>

The implanted support had a smooth internal surface observed with the naked eye. HE staining indicated complete absorption of PGA and PLA and a tissue structure comparable to normal blood vessels.

The vascular endothelial cells were studied by Factor VIII staining and the vascular smooth muscle cells were studied by α-SMA immunostaining. α-SMA immunostaining was conducted using antibodies for α-SMA. The Factor VIII immunostaining indicated a monolayer of continuous vascular endothelial cells and the α-SMA immunostaining indicated the smooth muscle cells aligned on the internal surface.

Moreover, the vascular elastic fiber was studied by elastica van Gieson staining. Elastic fiber was observed in an internal layer of a blood vessel.

<In Vivo: Six Months After Implantation>

As observed two months after implantation, a monolayer of continuous vascular endothelial cells were observed by Factor VIII immunostaining. The morphology of the smooth muscle cells was clearly observed as compared to what was observed two months after implantation. α-SMA immunostaining indicated that the smooth muscle cells were aligned on the internal surface and had substantially the same morphology as in normal blood vessels. Elastica van Gieson staining indicated that a larger amount of vascular elastic fiber was observed in an internal layer of a blood vessel than at two months after implantation. The presence or absence of calcification in blood vessels was studied by von Kossa staining. A positive reaction was not observed in the implanted composite film and blood vessels in its vicinity, i.e., calcification was not observed.

### Example 17

### Implantation into Heart

Next, the support (with collagen and without collagen) produced in Example 10 was implanted into the infarcted heart of rats.

<Myocardial Infarction Rat Model>

Male Lewis rat models were used in Example 17. Animals were cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health.

Acute myocardial infarction was induced as described in Weisman H. F., Bush D. E., Mannisi J. A., et al., Cellular Mechanism of Myocardial Infarct Expansion, Circulation, 1988; 78: 186-201. Briefly, rats (300 g, 8 weeks old) were anesthetized with sodium pentobarbital, followed by positive pressure breathing. In order to rat myocardial infarction models, a left 4th intercostal space thoracotomy was used and the left coronary artery was completely ligated with an 8-0 polypropylene thread at a distance of 3 mm from the root of the left coronary artery.

<Implantation of Support>

The myocardial infarcted rats were anesthetized, followed by a left 5th intercostal space thoracotomy to expose the heart. The rats were divided into two groups depending on whether or not the material was implanted into the myocardial infarction region: group C (no treatment group, n=5); and group S (support implanted group, n=5). The support was implanted directly to the infarction site after 2 weeks of ligation of the left anterior descending artery.

<Measurement of Cardiac Function of Rat>

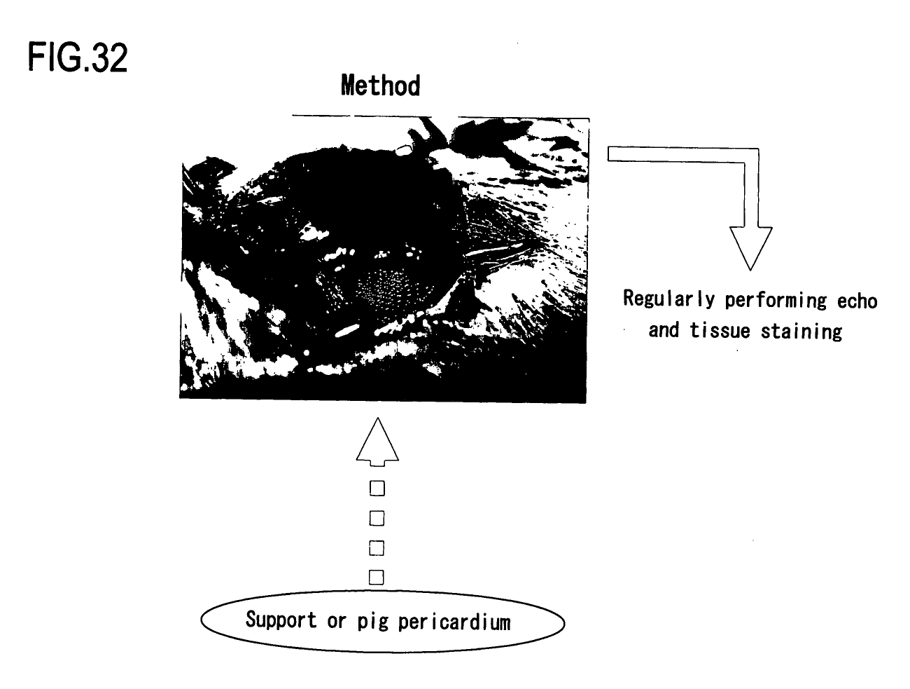

The cardiac function of the rats was measured after 2 weeks after production of the infarction model or 4 or 8 weeks after implantation using a heart ultrasonic instrument (manufactured by SONOS 5500, Agilent Technologies) (FIG. 32). A 12-MHz transducer was used to draw a minor axis image at a position such that the left ventricle indicated the maximum diameter viewed from the left. In a B mode, a left ventricular end-systolic area was measured. In an M mode, a left ventricular end-diastolic diameter (LVDd), a left ventricular end-systolic diameter (LVDs), and a left ventricular anterior wall thickness (LVAWTh) were measured. Thereby, a left ventricular ejection fraction (LVEF) and a left ventricular fractional shortening (LVFS) were calculated.

<Histological Analysis>

The heart was extracted 4 or 8 weeks after implantation of the support of the present invention and was sectioned along the minor axis. The sections were immersed in 10% formaldehyde solution, followed by paraffin embedding. The sample was sliced, followed by hematoxylin-eosin staining and Masson's Trichrome staining. Masson's Trichrome staining was conducted as detailed below. Meanwhile, some slices were frozen, followed by Factor VIII immunostaining (FIG. 32).

<Masson's Trichrome Staining>

Masson's Trichrome staining is performed as follows. Masson's Trichrome staining stains nuclei with iron hematoxylin. Thereafter, small pigment molecules (acid fuchsin, xylidine ponceau) having a high diffusion rate enter cell reticular channels, and next, large pigment molecules (aniline blue) having a low diffusion rate enter the rough structure of collagen fibers, thereby staining the cell with blue.

Masson's Trichrome staining uses the following reagents.

A) Dye mordant

aqueous 10% trichloroacetic acid solution 1 part

aqueous 10% potassium dichromate solution 1 part

B) Weigert's iron hematoxylin solution (equal amounts of solution 1 and solution 2 are mixed in use)

solution 1

| | |
|---|---|
| hematoxylin | 1 g |
| 100% ethanol | 100 ml |

solution 2

| | |
|---|---|
| ferric chloride | 2.0 g |
| hydrochloric acid (25%) | 1 ml |
| distilled water | 95 ml |

C) 1% hydrochloric acid 70% alcohol

D) I solution

| | |
|---|---|
| 1% Biebrich red | 90 ml |
| 1% acid fuchsin | 10 ml |
| acetic acid | 1 ml |

E) II solution

| | |
|---|---|
| phosphomolybdic acid | 5 g |
| phosphotungstic acid | 5 g |
| distilled water | 200 ml |

F) III solution

| | |
|---|---|
| aniline blue | 2.5 g |
| acetic acid | 2 ml |
| distilled water | 100 ml |

G) 1% acetic acid water
Procedure for Masson's Trichrome Staining:
1. deparaffinization, washing with water, distilled water;
2. mordanting (10 to 15 min):
3. washing with water (5 min);
4. Weigert's iron hematoxylin solution (5 min);
5. light washing with water:
6. separation with 1% hydrochloric acid 70% alcohol;
7. color development, washing with water (10 min);
8. distilled water;
9. I solution (2 to 5 min);
10. light washing with water:
11. II solution (30 mm or more);
12. light washing with water;
13. III solution (5 min);
14. light washing with water;
15. 1% acetic acid/water (5 min);
16. washing with water (quick); and
17. dehydration, clearing, mounting.

With Masson's Trichrome staining, collagen fiber, reticular fiber and glomerular basement membrane are vividly blue stained, nuclei are black-violet stained, plasma is pale-red stained, erythrocytes are orange-yellow to deep-red stained, mucus is blue stained, basophilic granules are blue stained and eosinphilic granules are red stained, and fibrin is red stained. Therefore, a blue-stained area can be calculated as a fibrous site. After treatment with a specific cytokine or growth factor, an antifibrosis action can be herein judged by determining whether or not a fibrous area is statistically significantly reduced.

<Results>

4 weeks after implantation, echocardiography was conducted. The ejection rate and the left ventricular fractional shortening were significantly improved in group S as compared to group C. Such an improvement was retained until at least 8 weeks after implantation.

<Histological Assessment>

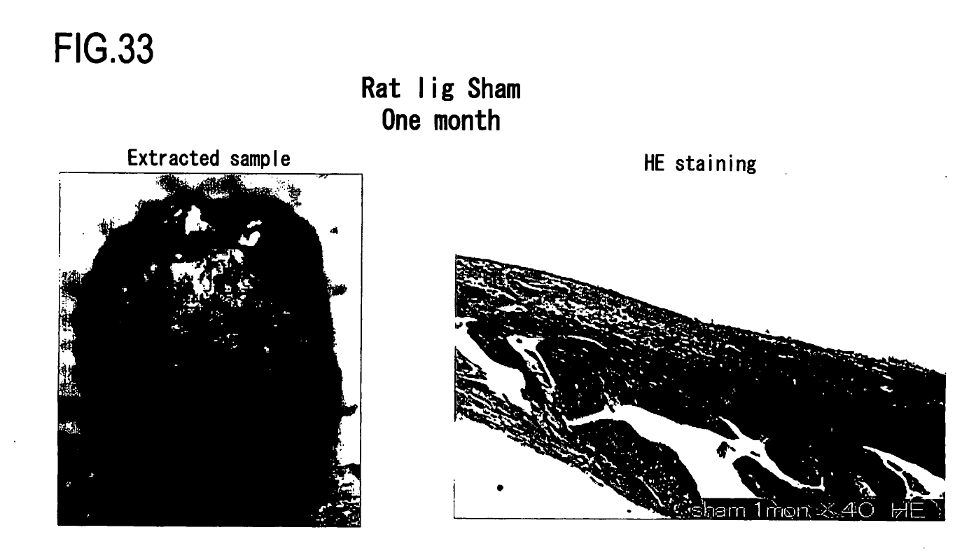

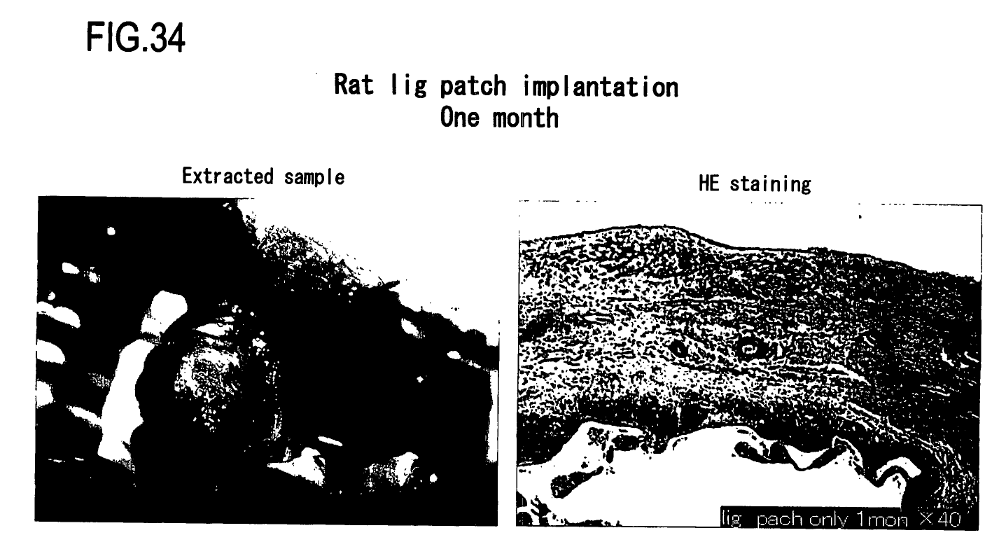

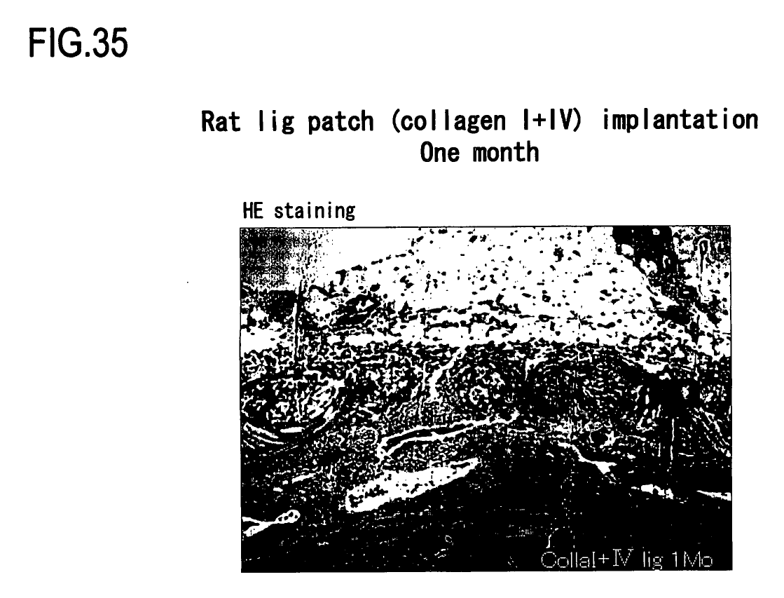

Group S had a significant increase in the thickness of the LV wall and a significant reduction in the LV cross section as compared to group C. The microscopic inspection revealed that a newly formed heart tissue compensated for a part of the LV wall suffering from infarction. This state is concretely shown in FIGS. 33 to 35. FIG. 33 shows a state of a control (without implantation) at the same stage as that in FIGS. 34 and 35. FIG. 34 shows a state of a support of the present invention (without a biological molecule) after one month of implantation. FIG. 35 shows a state of a support of the present invention (with type IV collagen and type I) after one month of implantation. As can be seen from the figures, the new formation of blood vessels and the vanishment of the support (patch) of the present invention were observed. This phenomenon was more significant in the support with type IV collagen and type I.

Therefore, it was demonstrated that a support of the present invention can provide an implant capable of being cellularized without self-reproducing material derived from organisms, such as a cell. Since such an effect was found when the support was used singly, it was demonstrated that a biocompatible support can be provided which overcomes the drawbacks of conventional knits and wovens.

### Example 18

### Demonstration of Cardiovascular Repair Material in Myocardial Infarction Rat Model

In Example 18, it was demonstrated that a tube-like support can also provide the effect of the present invention. A knit-woven composite support comprising a knit of poly(glycolic acid) and a woven of poly(glycolic acid) or poly(L-lactic acid) was produced. Poly(glycolic acid) and poly(L-lactic acid) are bioabsorbable polymers. A collagen microsponge was provided on the knit-woven composite support by crosslinking treatment. In addition, type I collagen and other extracellular matrices, i.e., type IV collagen and laminin were introduced into the support to produce a cardiovascular repair material.

<Rat Myocardial Infarction Model>

Male Lewis rats were used in Example 18. Animals were cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health. Acute myocardial infarction was induced as described in Weisman H. F., Bush D. E., Mannisi J. A., et al., Cellular Mechanism of Myocardial Infarct Expansion, Circulation, 1988; 78: 186-201. Briefly, rats (300 g, 8 weeks old) were anesthetized with sodium pentobarbital, followed by positive pressure breathing. In rat myocardial infarction models, a left 4th intercostal space thoracotomy was used and the left coronary artery was completely ligated with an 8-0 polypropylene thread at a distance of 3 mm from the root of the left coronary artery.

<Implantation>

The recipient rats were anesthetized and a left 5th intercostal space thoractomy was used to expose the heart. The rats were divided into three groups according to the material implanted into the myocardial infarction region: group C (no treatment group, n=5); group S1 (repair material-only implanted group, n=5): and group S2 (repair material+type I collagen+type IV collagen+laminin implanted group, n=5). The cardiovascular repair material was implanted directly into an infarction site two weeks after the left anterior descending artery had been ligated.

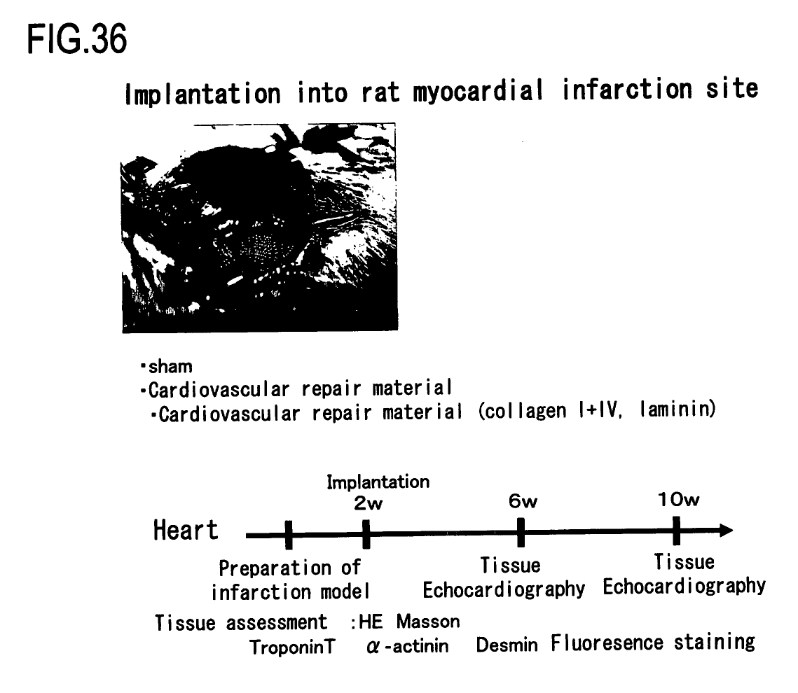

A state of the implanted site is shown in FIG. 36.

<Measurement of Cardiac Function of Rat>

The cardiac function of the rats was measured after 2 weeks after production of the infarction model or 4 or 8 weeks after implantation using a heart ultrasonic instrument (manufactured by SONOS 5500, Agilent Technologies). A 12-MHz transducer was used to draw a minor axis image at a position such that the left ventricle indicated the maximum diameter viewed from the left. In a B mode, a left ventricular end-systolic area was measured. In an M mode, a left ventricular end-diastolic diameter (LVDd), a left ventricular end-systolic diameter (LVDs), and a left ventricular anterior wall thickness (LVAWTh) were measured. Thereby, a left ventricular ejection fraction (LVEF) and a left ventricular fractional shortening (LVFS) were calculated.

<Histological Analysis>

The heart was extracted 4 or 8 weeks after implantation and was sectioned along the minor axis. The sections were immersed in 10% formaldehyde solution, followed by paraffin embedding. The sample was sliced, followed by hematoxylin-eosin staining and Masson's Trichrome staining. Meanwhile, some slices were frozen, followed by Desmin, Actinin, and TroponinT staining.

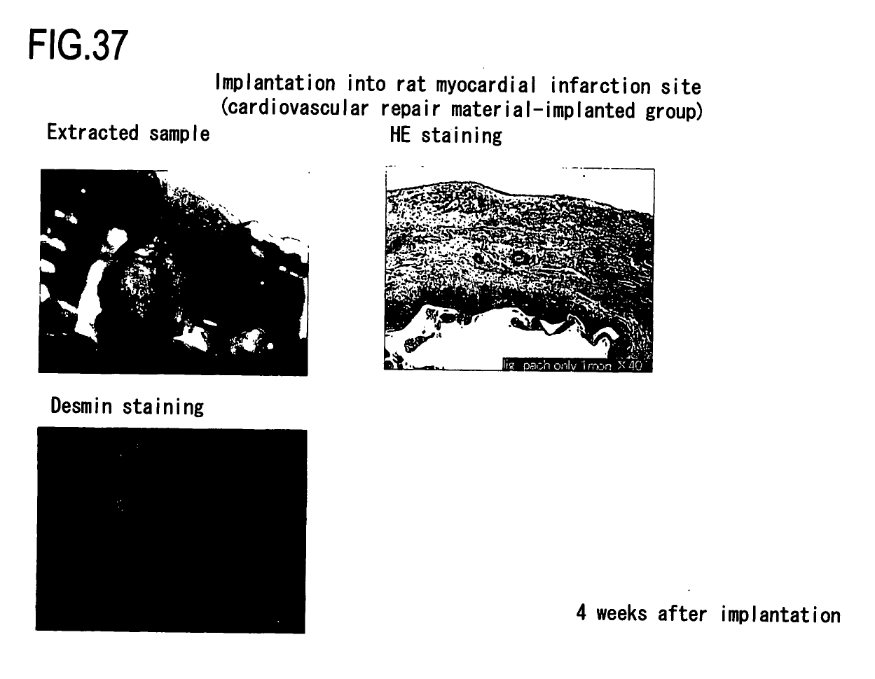

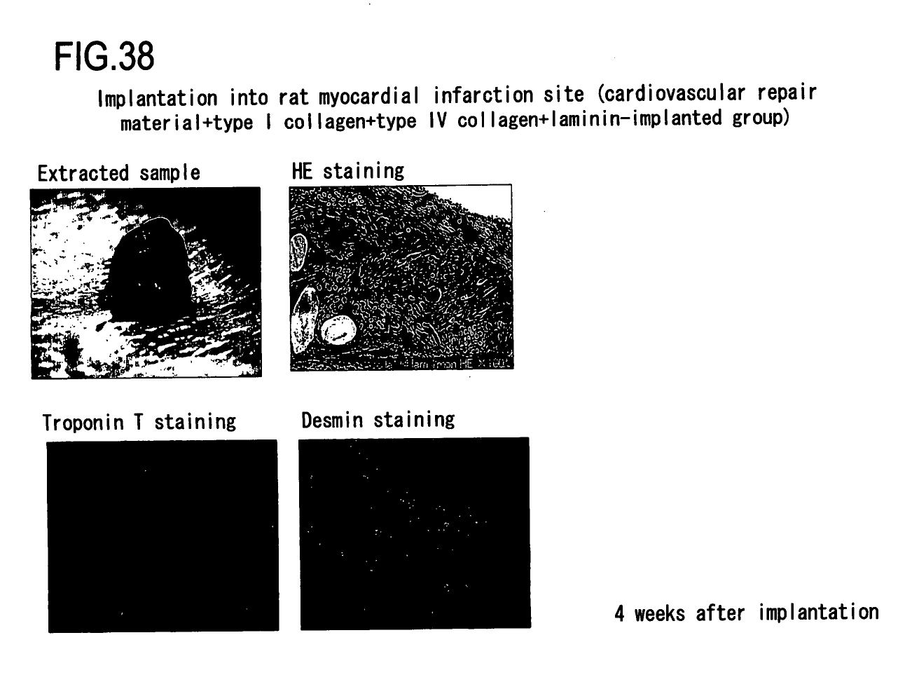

FIG. 37 shows a photograph of an extracted sample and results of HE staining and Desmin staining (the cardiovascular repair material implanted group). FIG. 38 shows a photograph of an extracted sample and results of HE staining, TroponinT staining, and Desmin staining (the cardiovascular repair material+type I collagen+type IV collagen+laminin implanted group).

<Results>

Photographs described in the results below show a combination of a poly(glycolic acid) knit and a poly(L-lactic acid) woven. A similar effect was seen in the case of a combination of a poly(glycolic acid) knit and a poly(glycolic acid) woven. Poly(L-lactic acid) seems to be sometimes preferable since it is difficult to degrade. However, the present invention is not limited to poly(L-lactic acid). Rather, it should be noted that both the above-described combinations could achieve the object of the present invention.

<Cardiac Function Assessment>

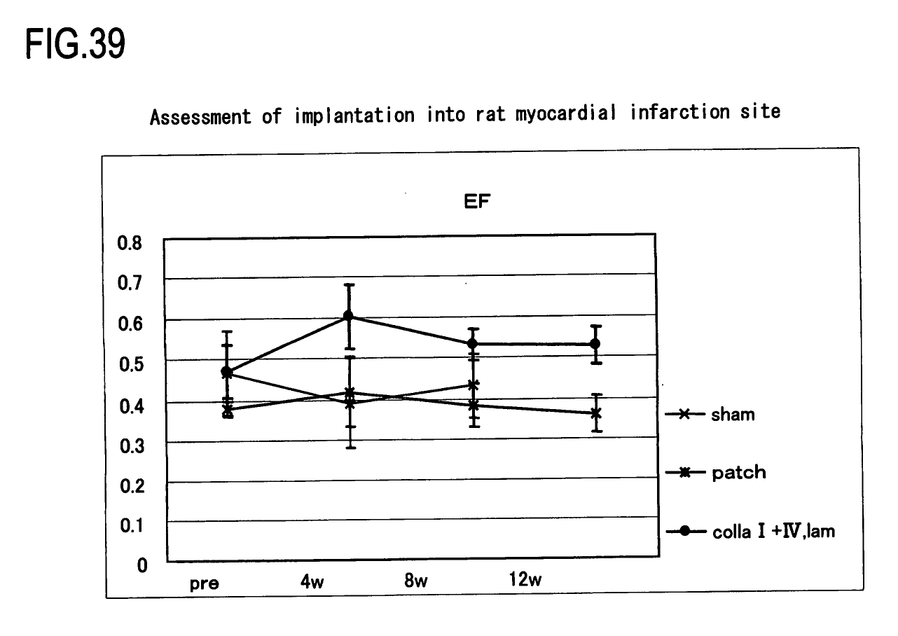

4 weeks after implantation, echocardiography was conducted. Group S2 had an ejection rate of 60%, while groups C and S1 had an ejection rate of 40% and 42%, respectively. Thus, the ejection rate was significantly improved in group S2 as compared to groups C and S1. Such an improvement was retained until at least 8 weeks after implantation. The results are shown in FIG. 39.

<Histological Assessment>

As can be clearly seen from FIGS. 37 and 38, group S2 had a significant increase in the thickness of the LV wall and a significant reduction in the LV cross section as compared to group C. The microscopic inspection revealed that there were cells which had not been provided in the repair material and that a newly formed heart tissue compensated for a part of the LV wall suffering from infarction. In group S2, when the regenerated tissue was immunohistologically stained (Desmin, Actinin, TroponinT staining), positive cells were observed.

### Example 19

### Use of Short Peptide)

In Example 19, it is demonstrated that a support on which a short peptide is applied can also provide the effect of the present invention. A knit-woven composite support comprising a knit of poly(glycolic acid) and a woven of poly(glycolic acid) or poly(L-lactic acid) is produced. Poly(glycolic acid) and poly(L-lactic acid) are bioabsorbable polymers. A collagen microsponge is provided on the knit-woven composite support by crosslinking treatment. In addition, a short peptide SVVYGLR (SEQ ID NO:1) is introduced into the support to produce a cardiovascular repair material. The short peptide SVVYGLR is known to have an action of angiogenesis as described in, for example, WO03/030925.

<Rat Myocardial Infarction Model>

Male Lewis rats are used in Example 19. Animals are cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health. Acute myocardial infarction is induced as described in Weisman H. F., Bush D. E., Mannisi J. A., et al., Cellular Mechanism of Myocardial Infarct Expansion, Circulation, 1988; 78: 186-201. Briefly, rats (300 g, 8 weeks old) are anesthetized with sodium pentobarbital, followed by positive pressure breathing. In order to rat myocardial infarction models, a left 4th intercostal space thoracotomy is used and the left coronary artery is completely ligated with an 8-0 polypropylene thread at a distance of 3 mm from the root of the left coronary artery.

<Implantation>

The recipient rats are anesthetized and a left 5th intercostal space thoractomy is used to expose the heart. The rats are divided into three groups according to the material implanted into the myocardial infarction region: group C (no treatment group, n=5); group S1 (repair material-only implanted group, n=5); and group S2 (repair material+short peptide implanted group, n=5). The cardiovascular repair material is implanted directly into an infarction site two weeks after the left anterior descending artery is ligated.

<Measurement of Cardiac Function of Rat>

The cardiac function of the rats is measured after 2 weeks after production of the infarction model or 4 or 8 weeks after implantation using a heart ultrasonic instrument (manufactured by SONOS 5500, Agilent Technologies). A 12-MHz transducer is used to draw a minor axis image at a position such that the left ventricle indicated the maximum diameter viewed from the left. In a B mode, a left ventricular end-systolic area is measured. In an M mode, a left ventricular end-diastolic diameter (LVDd), a left ventricular end-systolic diameter (LVDs), and a left ventricular anterior wall thickness (LVAWTh) are measured. Thereby, a left ventricular ejection fraction (LVEF) and a left ventricular fractional shortening (LVFS) are calculated.

<Histological Analysis>

The heart is extracted 4 or 8 weeks after implantation and is sectioned along the minor axis. The sections were immersed in 10% formaldehyde solution, followed by paraffin embedding. The sample is sliced, followed by hematoxylin-eosin staining and Masson's Trichrome staining. Meanwhile, some slices are frozen, followed by Desmin, Actinin, and TroponinT staining.

<Results>

<Cardiac Function Assessment>

4 weeks after implantation, echocardiography is conducted. The ejection rate is significantly improved in group S2 as compared to groups C and S1. Such an improvement is retained until at least 8 weeks after implantation.

<Histological Assessment>

Group S2 has a significant increase in the thickness of the LV wall and a significant reduction in the LV cross section as compared to group C. The microscopic inspection reveals that there are cells which have not been provided in the repair material and that a newly formed heart tissue compensates for a part of the LV wall suffering from infarction. In group S2, when the regenerated tissue is immunohistologically stained (Desmin, Actinin, TroponinT staining), positive cells are observed.

A combination of a poly(glycolic acid) knit and a poly(L-lactic acid) woven and a combination of a poly(glycolic acid) knit and a poly(glycolic acid) woven provide similar effects.

### Example 20

### Use of Cytokine

In Example 20, it is demonstrated that a support on which a cytokine is applied can also provide the effect of the present invention. A knit-woven composite support comprising a knit of poly(glycolic acid) and a woven of poly(glycolic acid) or poly(L-lactic acid) is produced. Poly(glycolic acid) and poly(L-lactic acid) are bioabsorbable polymers. A collagen microsponge is provided on the knit-woven composite support by crosslinking treatment In addition, a cytokine HGF (available from Toyobo) is introduced into the support to produce a cardiovascular repair material. HGF was identified as a hepatocyto growth factor and is also known as a factor capable of contributing to regeneration of heart, blood vessel, and the like.

<Rat Myocardial Infarction Model>

Male Lewis rats are used in Example 20. Animals are cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health. Acute myocardial infarction is induced as described in Weisman H. F., Bush D. E., Mannisi J. A., et al., Cellular Mechanism of Myocardial Infarct Expansion, Circulation, 1988: 78: 186-201. Briefly, rats (300 g, 8 weeks old) are anesthetized with sodium pentobarbital, followed by positive pressure breathing. In order to rat myocardial infarction models, a left 4th intercostal space thoracotomy is used and the left coronary artery is completely ligated with an 8-0 polypropylene thread at a distance of 3 mm from the root of the left coronary artery.

<Implantation>

The recipient rats are anesthetized and a left 5th intercostal space thoractomy is used to expose the heart. The rats are divided into three groups according to the material implanted into the myocardial infarction region: group C (no treatment group, n=5); group S1 (repair material-only implanted group, n=5); and group S2 (repair material+HGF implanted group, n=5). The cardiovascular repair material is implanted directly into an infarction site two weeks after the left anterior descending artery is ligated.

<Measurement of Cardiac Function of Rat>

The cardiac function of the rats is measured after 2 weeks after production of the infarction model or 4 or 8 weeks after implantation using a heart ultrasonic instrument (manufactured by SONOS 5500, Agilent Technologies). A 12-MHz transducer is used to draw a minor axis image at a position such that the left ventricle indicated the maximum diameter viewed from the left. In a B mode, a left ventricular end-systolic area is measured. In an M mode, a left ventricular end-diastolic diameter (LVDd), a left ventricular end-systolic diameter (LVDs), and a left ventricular anterior wall thickness (LVAWTh) are measured. Thereby, a left ventricular ejection fraction (LVEF) and a left ventricular fractional shortening (LVFS) are calculated.

<Histological Analysis>

The heart is extracted 4 or 8 weeks after implantation and is sectioned along the minor axis. The sections were immersed in 10% formaldehyde solution, followed by paraffin embedding. The sample is sliced, followed by hematoxylin-eosin staining and Masson's Trichrome staining. Meanwhile, some slices are frozen, followed by Desmin, Actinin, and TroponinT staining.

<Results>

<Cardiac Function Assessment>

4 weeks after implantation, echocardiography is conducted. The ejection rate is significantly improved in group S2 as compared to groups C and S1.

<Histological Assessment>

Group S2 has a significant increase in the thickness of the LV wall and a significant reduction in the LV cross section as compared to group C. The microscopic inspection reveals that there are cells which have not been provided in the repair material and that a newly formed heart tissue compensates for a part of the LV wall suffering from infarction. In group S2, when the regenerated tissue is immunohistologically stained (Desmin, Actinin, TroponinT staining), positive cells are observed.

A combination of a poly(glycolic acid) knit and a poly(L-lactic acid) woven and a combination of a poly(glycolic acid) knit and a poly(glycolic acid) woven provide similar effects.

### Example 21

### Use of Another Cytokine

In Example 21, it is demonstrated that a support on which a cytokine is applied can also provide the effect of the present invention. A knit-woven composite support comprising a knit of poly(glycolic acid) and a woven of poly(glycolic acid) or poly(L-lactic acid) is produced. Poly(glycolic acid) and poly(L-lactic acid) are bioabsorbable polymers. A collagen microsponge is provided on the knit-woven composite support by crosslinking treatment. In addition, a cytokine VEGF (available from Biosourse International) is introduced into the support to produce a cardiovascular repair material. VEGF is known as a factor capable of contributing to regeneration of heart, blood vessel, and the like.

<Rat Myocardial Infarction Model>

Male Lewis rats are used in Example 21. Animals are cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health. Acute myocardial infarction is induced as described in Weisman H. F., Bush D. E., Mannisi J. A., et al., Cellular Mechanism of Myocardial Infarct Expansion, Circulation, 1988; 78: 186-201. Briefly, rats (300 g, 8 weeks old) are anesthetized with sodium pentobarbital, followed by positive pressure breathing. In order to rat myocardial infarction models, a left 4th intercostal space thoracotomy is used and the left coronary artery is completely ligated with an 8-0 polypropylene thread at a distance of 3 mm from the root of the left coronary artery.

<Implantation>

The recipient rats are anesthetized and a left 5th intercostal space thoractomy is used to expose the heart. The rats are divided into three groups according to the material implanted into the myocardial infarction region: group C (no treatment group, n=5); group S1 (repair material-only implanted group, n=5); and group S2 (repair material+VEGF implanted group, n=5). The cardiovascular repair material is implanted directly into an infarction site two weeks after the left anterior descending artery is ligated.

<Measurement of Cardiac Function of Rat>

The cardiac function of the rats is measured after 2 weeks after production of the infarction model or 4 or 8 weeks after implantation using a heart ultrasonic instrument (manufactured by SONOS 5500, Agilent Technologies). A 12-MHz transducer is used to draw a minor axis image at a position such that the left ventricle indicated the maximum diameter viewed from the left. In a B mode, a left ventricular end-systolic area is measured. In an M mode, a left ventricular end-diastolic diameter (LVDd), a left ventricular end-systolic diameter (LVDs), and a left ventricular anterior wall thickness (LVAWTh) are measured. Thereby, a left ventricular ejection fraction (LVEF) and a left ventricular fractional shortening (LVFS) are calculated.

<Histological Analysis>

The heart is extracted 4 or 8 weeks after implantation and is sectioned along the minor axis. The sections were immersed in 10% formaldehyde solution, followed by paraffin embedding. The sample is sliced, followed by hematoxylin-eosin staining and Masson's Trichrome staining. Meanwhile, some slices are frozen, followed by Desmin, Actinin, and TroponinT staining.

<Results>

<Cardiac Function Assessment>

4 weeks after implantation, echocardiography is conducted. The ejection rate is significantly improved in group S2 as compared to groups C and S1.

<Histological Assessment>

Group S2 has a significant increase in the thickness of the LV wall and a significant reduction in the LV cross section as compared to group C. The microscopic inspection reveals that there are cells which have not been provided in the repair material and that a newly formed heart tissue compensates for a part of the LV wall suffering from infarction. In group S2, when the regenerated tissue is immunohistologically stained (Desmin, Actinin, TroponinT staining), positive cells are observed.

A combination of a poly(glycolic acid) knit and a poly(L-lactic acid) woven and a combination of a poly(glycolic acid) knit and a poly(glycolic acid) woven provide similar effects.

### Example 22

### Use of Cytokine and Extracellular Matrix

In Example 22, it is demonstrated that a support on which a combination of a cytokine and an extracellular matrix is applied can also provide the effect of the present invention A knit-woven composite support comprising a knit of poly(glycolic acid) and a woven of poly(glycolic acid) or poly(L-lactic acid) is produced. Poly(glycolic acid) and poly(L-lactic acid) are bioabsorbable polymers. A collagen microsponge is provided on the knit-woven composite support by crosslinking treatment. In addition, a cytokine HGF (available from Toyobo) and type I collagen (extracellular matrix) are introduced into the support to produce a cardiovascular repair material. The collagen is used as as in the above-described examples.

<Rat Myocardial Infarction Model>

Male Lewis rats are used in Example 20. Animals are cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health. Acute myocardial infarction is induced as described in Weisman H. F., Bush D. E., Mannisi J. A., et al., Cellular Mechanism of Myocardial Infarct Expansion, Circulation, 1988; 78: 186-201. Briefly, rats (300 g, 8 weeks old) are anesthetized with sodium pentobarbital, followed by positive pressure breathing. In order to rat myocardial infarction models, a left 4th intercostal space thoracotomy is used and the left coronary artery is completely ligated with an 8-0 polypropylene thread at a distance of 3 mm from the root of the left coronary artery.

<Implantation>

The recipient rats are anesthetized and a left 5th intercostal space thoractomy is used to expose the heart. The rats are divided into three groups according to the material implanted into the myocardial infarction region: group C (no treatment group, n=5); group S1 (repair material-only implanted group, n=5): and group S2 (repair material+HGF+type I collagen implanted group, n=5). The cardiovascular repair material is implanted directly into an infarction site two weeks after the left anterior descending artery is ligated.

<Measurement of Cardiac Function of Rat>

The cardiac function of the rats is measured after 2 weeks after production of the infarction model or 4 or 8 weeks after implantation using a heart ultrasonic instrument (manufactured by SONOS 5500, Agilent Technologies). A 12-MHz transducer is used to draw a minor axis image at a position such that the left ventricle indicated the maximum diameter viewed from the left. In a B mode, a left ventricular end-systolic area is measured. In an M mode, a left ventricular end-diastolic diameter (LVDd), a left ventricular end-systolic diameter (LVDs), and a left ventricular anterior wall thickness (LVAWTh) are measured. Thereby, a left ventricular ejection fraction (LVEF) and a left ventricular fractional shortening (LVFS) are calculated.

<Histological Analysis>

The heart is extracted 4 or 8 weeks after implantation and is sectioned along the minor axis. The sections were immersed in 10% formaldehyde solution, followed by paraffin embedding. The sample is sliced, followed by hematoxylin-eosin staining and Masson's Trichrome staining. Meanwhile, some slices are frozen, followed by Desmin, Actinin, and TroponinT staining.

<Results>

<Cardiac Function Assessment>

4 weeks after implantation, echocardiography is conducted. The ejection rate is significantly improved in group S2 as compared to groups C and S1.

<Histological Assessment>

Group S2 has a significant increase in the thickness of the LV wall and a significant reduction in the LV cross section as compared to group C. The microscopic inspection reveals that there are cells which have not been provided in the repair material and that a newly formed heart tissue compensates for a part of the LV wall suffering from infarction. In group S2, when the regenerated tissue is immunohistologically stained (Desmin, Actinin, TroponinT staining), positive cells are observed.

In general, a combination of an extracellular matrix and a cytokine has a higher level of effect than when the cytokine or the extracellular matrix is used singly.

A combination of a poly(glycolic acid) knit and a poly(L-lactic acid) woven and a combination of a poly(glycolic acid) knit and a poly(glycolic acid) woven provide similar effects.

### Example 23

### Use of Another Biological Molecule

In Example 23, it is demonstrated that a support on which another biological molecule is applied can also provide the effect of the present invention. A knit-woven composite support comprising a knit of poly(glycolic acid) and a woven of poly(glycolic acid) or poly(L-lactic acid) is produced. Poly(glycolic acid) and poly(L-lactic acid) are bioabsorbable polymers. A collagen microsponge is provided on the knit-woven composite support by crosslinking treatment. In addition, laminin (Becton, Dickinson and Company); angiopoietin (R&D Systems); HGF (PeproTech, Inc.); FGF (fibroblast growth factor, trade name: Fibrast spray (Kaken Pharmaceutical); G-CSF (granulocyte colony stimulating factor, trade name: GRAN (Kirin Brewery); SDF-1 (Decton, Dickinson and Company), TNF-α (Peprotech, Inc.), and IL1-β (Peprotech, Inc.) are introduced singly into the support to produce respective cardiovascular repair materials. These biological molecules are known as factors capable of contributing to regeneration of cardiac muscle, and the like.

<Rat Myocardial Infarction Model>

Male Lewis rats are used in Example 21. Animals are cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health. Acute myocardial infarction is induced as described in Weisman H. F., Bush D. E., Mannisi J. A., et al., Cellular Mechanism of Myocardial Infarct Expansion, Circulation, 1988; 78: 186-201. Briefly, rats (300 g, 8 weeks old) are anesthetized with sodium pentobarbital, followed by positive pressure breathing. In order to rat myocardial infarction models, a left 4th intercostal space thoracotomy is used and the left coronary artery is completely ligated with an 8-0 polypropylene thread at a distance of 3 mm from the root of the left coronary artery.

<Implantation>

The recipient rats are anesthetized and a left 5th intercostal space thoractomy is used to expose the heart. The rats are divided into four groups according to the material implanted into the myocardial infarction region: group C (no treatment group, n=5); group S1 (repair material-only implanted group, n=5); group S2 (repair material+SDF-1, TNF-α, or IL1-β implanted group, n=5); and group S3 (repair material+collagen+SDF-1, TNF-α, or IL1-β implanted group, n=5). The cardiovascular repair material is implanted directly into an infarction site two weeks after the left anterior descending artery is ligated.

<Measurement of Cardiac Function of Rat>

The cardiac function of the rats is measured after 2 weeks after production of the infarction model or 4 or 8 weeks after implantation using a heart ultrasonic instrument (manufactured by SONOS 5500, Agilent Technologies). A 12-MHz transducer is used to draw a minor axis image at a position such that the left ventricle indicated the maximum diameter viewed from the left. In a B mode, a left ventricular end-systolic area is measured. In an M mode, a left ventricular end-diastolic diameter (LVDd), a left ventricular end-systolic diameter (LVDs), and a left ventricular anterior wall thickness (LVAWTh) are measured. Thereby, a left ventricular ejection fraction (LVEF) and a left ventricular fractional shortening (LVFS) are calculated.

<Histological Analysis>

The heart is extracted 4 or 8 weeks after implantation and is sectioned along the minor axis. The sections were immersed in 10% formaldehyde solution, followed by paraffin embedding. The sample is sliced, followed by hematoxylin-eosin staining and Masson's Trichrome staining. Meanwhile, some slices are frozen, followed by Desmin, Actinin, and TroponinT staining.

<Results>

<Cardiac Function Assessment>

4 weeks after implantation, echocardiography is conducted. The ejection rate is significantly improved in groups S2 and S3 as compared to groups C and S1.

<Histological Assessment>

Groups S1 to S3 have a significant increase in the thickness of the LV wall and a significant reduction in the LV cross section as compared to group C. The microscopic inspection reveals that there are cells which have not been provided in the repair material and that a newly formed heart tissue compensates for a part of the LV wall suffering from infarction. In groups S1 to S3, when the regenerated tissue is immunohistologically stained (Desmin, Actinin, TroponinT staining), positive cells are observed.

A combination of a poly(glycolic acid) knit and a poly(L-lactic acid) woven and a combination of a poly(glycolic acid) knit and a poly(glycolic acid) woven provide similar effects.

### Example 24

### Demonstration of Cardiovascular Repair Material in Rat Dorsal Implantation Model

In Example 24, it was demonstrated that a tube-like support can also provide the effect of the present invention. A knit-woven composite support comprising a knit of poly(glycolic acid) and a woven of poly(glycolic acid) or poly(L-lactic acid) was produced. Poly(glycolic acid) and poly(L-lactic acid) are bioabsorbable polymers. A collagen microsponge was provided on the knit-woven composite support by crosslinking treatment. In addition, type I collagen and other extracellular matrices, i.e., type IV collagen, laminin and HGF were introduced into the support to produce a cardiovascular repair material.

<Rat Dorsal Implantation Model>

Male Lewis rats were used in Example 18. Animals were cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health. Rats (300 g, 8 weeks old) were anesthetized with sodium pentobarbital, followed by positive pressure breathing. The rats were divided into three groups according to the material implanted into the myocardial infarction region: group C (repair material-only implanted group, n=5); group S1 (repair material+type I collagen+HGF implanted group, n=5); and group S2 (repair material+type I collagen+type IV collagen+laminin implanted group, n=5).

The experiment was conducted based on Shimizu T., Yamato M., Akutsu T., et al., Circ. Res., 2002, Feb. 22; 90 (3): e40.

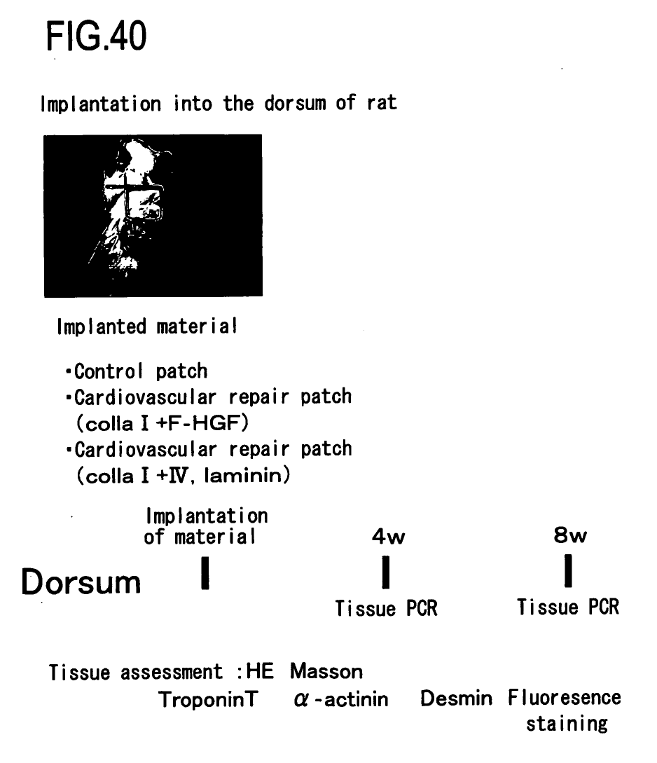

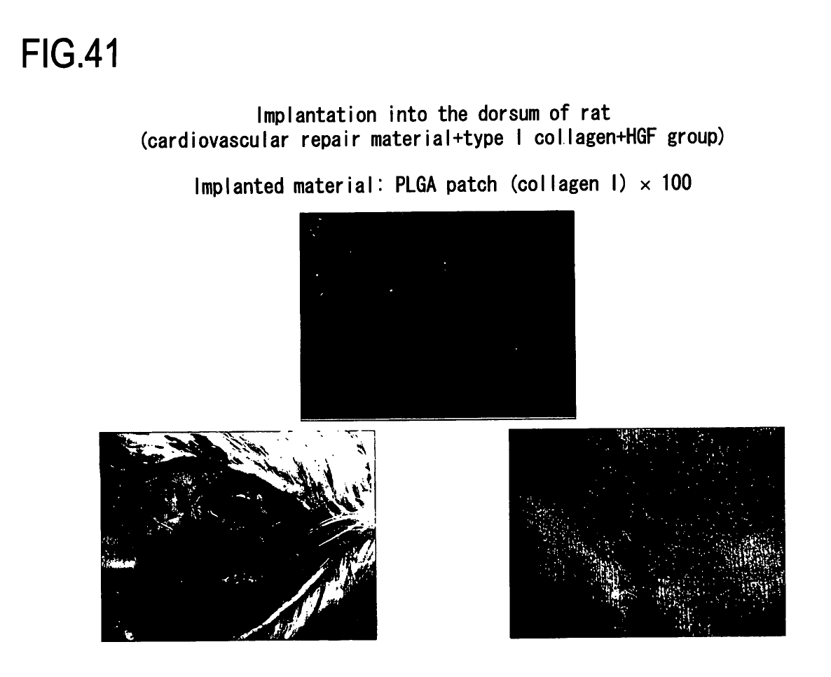

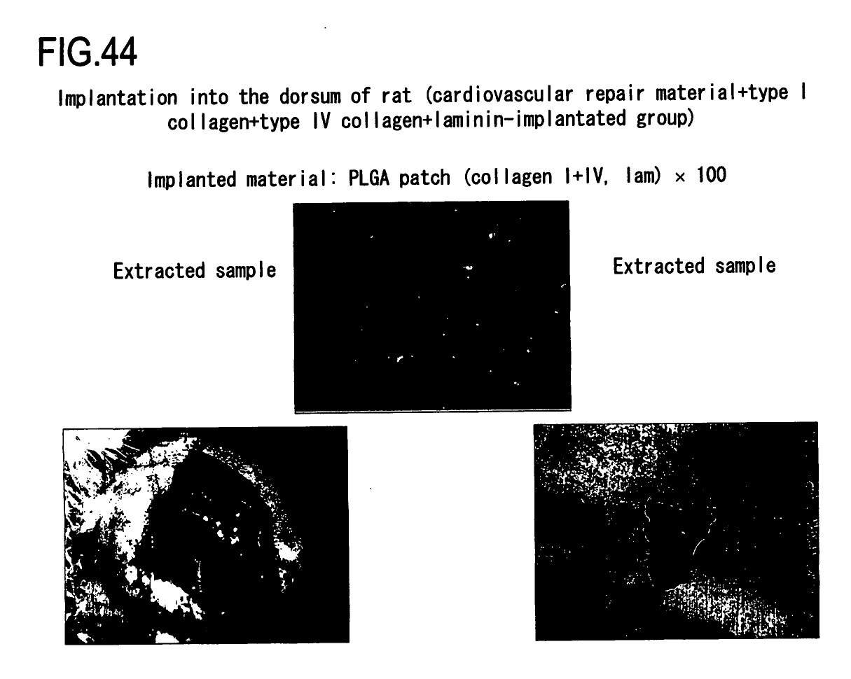

A protocol used in the experiment is shown in FIG. 40. FIG. 41 shows a state of rat dorsal implantation (the repair material+type I collagen+HGF implanted group). FIG. 44 shows a state of rat dorsal implantation (the repair material+type I collagen+type IV collagen implanted group).

<Histological Analysis>

The heart was extracted 4 or 8 weeks after implantation and was sectioned along the minor axis. The sections were immersed in 10% formaldehyde solution, followed by paraffin embedding. The sample was sliced, followed by hematoxylin-eosin staining and Masson's Trichrome staining. Meanwhile, some slices were frozen, followed by Desmin, Actinin, and TroponinT staining.

<Quantification PCR>

The heart was extracted 4 or 8 weeks after implantation, followed by quantification PCR for cardiac actin, α-MBC and β-MBC. In quantification PCR, the following primers and probes for quantification are used.

CardiacActin

| | |
|---|---|
| 5′ primer | |
| ACC CTG GAA TTG CTG ATC GTA TG | (SEQ ID NO:2) |
| 3′ primer | |
| TGT CGT CCT GAG TGT AAG GTA GCC | (SEQ ID NO:3) |
| probe | |
| AAA TTA CCG CAC TGG CTC CCA GCA | (SEQ ID NO:4) |
| α-MHC | |
| 5′ primer | |
| TAG AAT AGC CTC AGA GGC CCA G | (SEQ ID NO:5) |
| 3′ primer | |
| GCT TCC GAG ACC GCT CTG TC | (SEQ ID NO:6) |
| probe | |
| CAG TCC GTG CCA ATG ACG ACC TGA A | (SEQ ID NO:7) |
| β-MHC | |
| 5′ primer | |
| TGC TGA AGG ACA CTC AAA TCC A | (SEQ ID NO:8) |
| 3′ primer | |
| GTT GAT GAG GCT GGT GTT CTG G | (SEQ ID NO:9) |
| probe | |
| ACG CAG TCC GTG CCA ATG ACG ACC | (SEQ ID NO:10) |

Quantifiation PCR was conducted as follows.

1. An extracted sample was preserved using RNA later (QIAGEN).

2. RNeasy Mini Kit (GIAGEN) was used to extract RNA.

3. RNase-Free DNase Set (QIAGEN) was used to treat DNA.

4. DNA treated using Omniscript RT Kit (QIAGEN) was subjected to a reverse transcription reaction.

5. TaqMan Universal PCR Master Mix (Roche) was used to conduct PCR.

<Results>

Photographs described in the results below show a combination of a poly(glycolic acid) knit and a poly(L-lactic acid) woven. A similar effect was seen in the case of a combination of a poly(glycolic acid) knit and a poly(glycolic acid) woven. Poly(L-lactic acid) seems to be sometimes preferable since it is difficult to degrade. However, the present invention is not limited to poly(L-lactic acid). Rather, it should be noted that both the above-described combinations could achieve the object of the present invention.

<Histological Assessment>

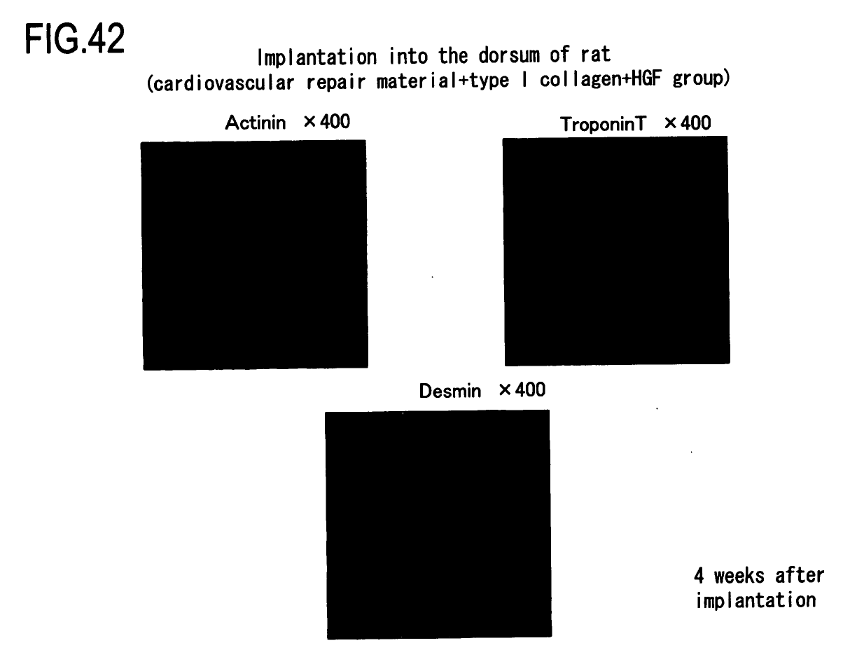

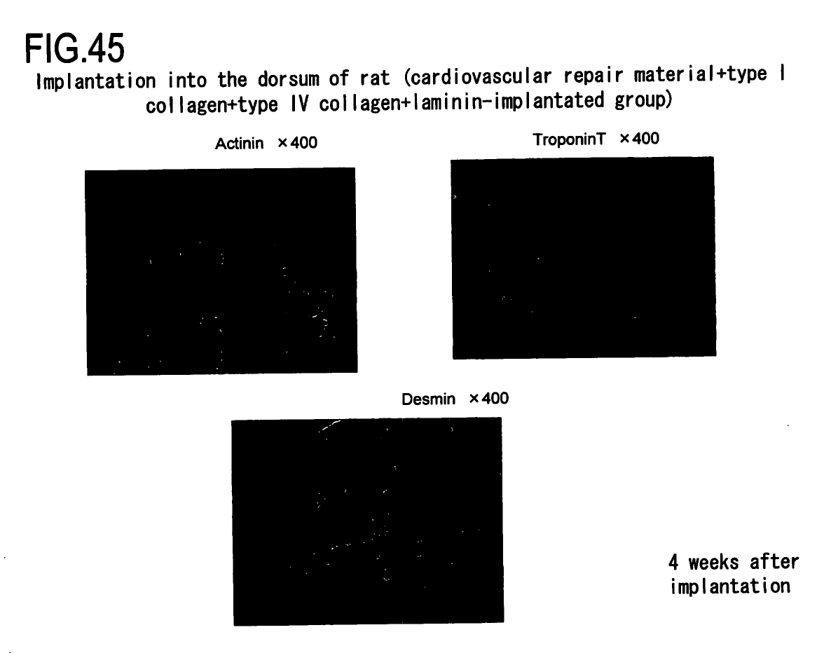

FIG. 42 (the repair material+type I collagen+HGF implanted group) and FIG. 45 (the repair material+type I collagen+type IV collagen implanted group) show frozen slices stained with Desmin, Actinin or TronponinT 4 weeks after implantation.

Group S2 had a significant increase in the thickness of the LV wall and a significant reduction in the LV cross section as compared to group C. The microscopic inspection revealed that there were cells which had not been provided in the repair material and that a newly formed heart tissue compensated for a part of the LV wall suffering from infarction. In group S2, when the regenerated tissue was immunohistologically stained (Desmin, Actinin, TroponinT staining), positive cells were observed.

<Quantification PCR>

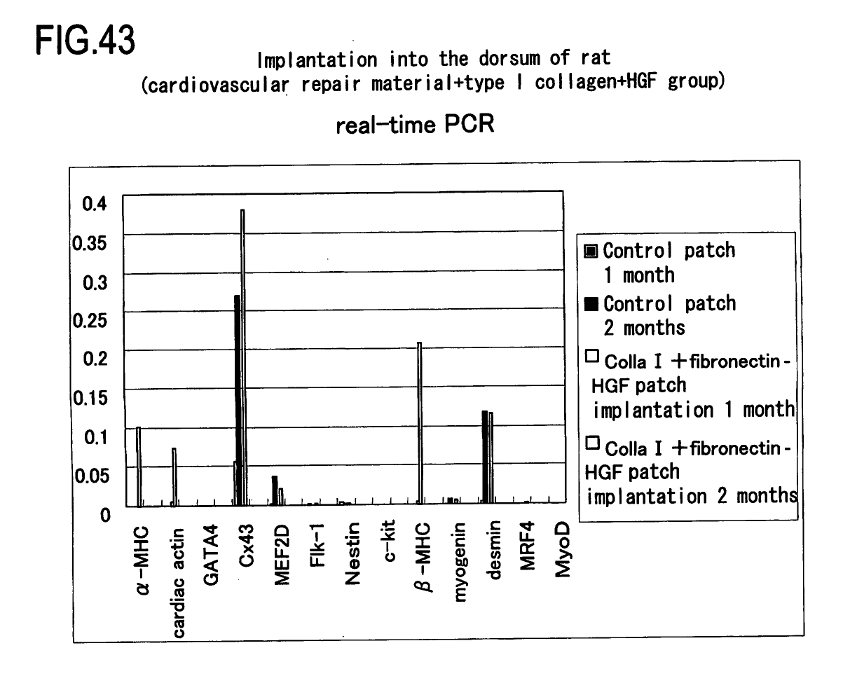

FIG. 43 shows results of various PCR for rat dorsal implantation (the cardiovascular repair material+type I collagen+HGF implanted group). Quantification PCR revealed that expression of cardiac actin, α-MHC and β-MHC was observed in groups S1 and S2 but not in group C.

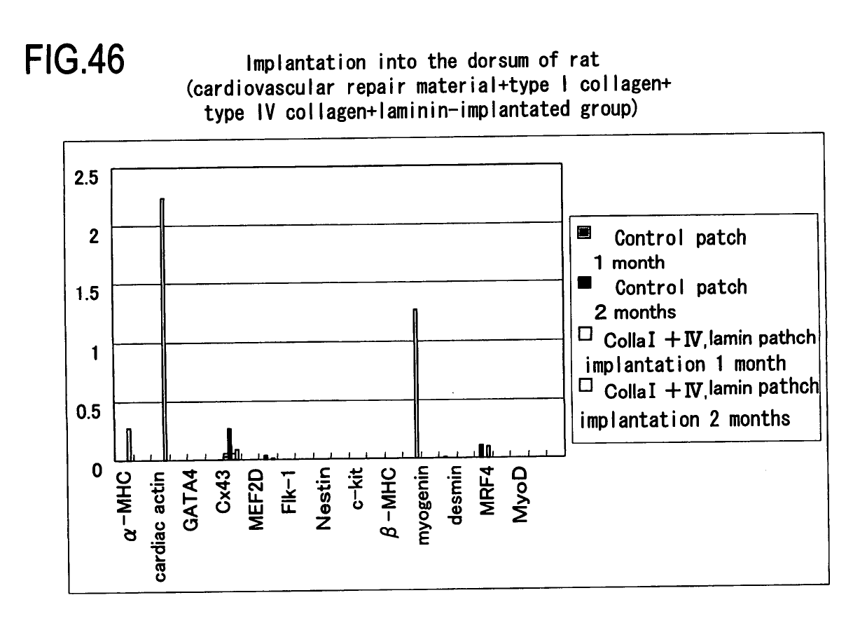

FIG. 46 shows results of various PCR for rat dorsal implantation (cardiovascular repair material+type I collagen+type IV collagen+HGF implanted group). Quantification PCR revealed that expression of cardiac actin, α-MHC and β-MHC was observed in groups S1 and S2 but not in group C.

The amount of expression was increased with an increase in the number of types of biological molecules.

### Example 25

### Demonstration of Cardiovascular Repair Material with Another molecule in Rat Dorsal Implantation Model

In Example 25, it was demonstrated that VEGF (PeproTech, Inc.); angiopoietin (R&D Systems); HGF (PeproTech, Inc.); FGF (fibroblast growth factor, trade name: Fibrast spray (Kaken Pharmaceutical); G-CSF (granulocyte colony stimulating factor, trade name: GRAN (Kirin Brewery): laminin (Becton, Dickinson and Company); SDF-1 (Decton, Dickinson and Company), TNF-α (Peprotech, Inc.), and IL1-β (Peprotech, Inc.) are used as biomolecules of the present invention to obtain the effect of the present invention. A knit-woven composite support comprising a knit of poly(glycolic acid) and a woven of poly(glycolic acid) or poly(L-lactic acid) is produced. The above-described three molecules are each introduced into a support to produce respective cardiovascular repair materials.

<Rat Dorsal Implantation Model>

Male Lewis rats are used in Example 18. Animals were cared for in the spirit of animal protection in accordance with “Principles of Laboratory Animal Care” prepared by the National Society for Medical Research and “Guide for the Care and Use of Laboratory Animals” (NIH Publication, No. 86-23, 1985, revised) prepared by the Institute of Laboratory Animal Resource and published by the National Institute of Health. Rats (300 g, 8 weeks old) are anesthetized with sodium pentobarbital, followed by positive pressure breathing. The rats are divided into three groups according to the material implanted into the myocardial infarction region: group C (repair material-only implanted group, n=5): group S1 (repair material+type I collagen+HGF implanted group, n=5); group S2 (repair material+VEGF; angiopoietin; HGF; FGF: G-CSF: or laminin implanted group, n=5 for each); and group S3 (repair material+collagen+VEGF: angiopoietin: HGF: FGF; G-CSF: or laminin implanted group, n=5 for each).

The experiment was conducted based on Shimizu T., Yamato M., Akutsu T., et al., Circ. Res., 2002, Feb. 22; 90(3): e40.

<Histological Analysis>

The heart was extracted 4 or 8 weeks after implantation and was sectioned along the minor axis. The sections were immersed in 10% formaldehyde solution, followed by paraffin embedding. The sample was sliced, followed by hematoxylin-eosin staining and Masson's Trichrome staining. Meanwhile, some slices were frozen, followed by Desmin, Actinin, and TroponinT staining.

<Quantification PCR>

The heart was extracted 4 or 8 weeks after implantation, followed by quantification PCR for cardiac actin, α-MBC and β-MBC as in Example 24.

<Results>

<Histological Assessment>

Group S1 to S3 have a significant increase in the thickness of the LV wall and a significant reduction in the LV cross section as compared to group C. The microscopic inspection reveals that there are cells which are not provided in the repair material and that a newly formed heart tissue compensates for a part of the LV wall suffering from infarction. In groups S1 to S3, when the regenerated tissue is immunohistologically stained (Desmin, Actinin, TroponinT staining), positive cells are observed. The effect of regeneration is more increased in the combination of collagen and the other cytokines than when the cytokines are used singly.

<Quantification PCR>

Quantification PCR reveals that expression of cardiac actin, α-MHC and β-MHC was observed in groups S1 to S3 but not in group C.

A combination of a poly(glycolic acid) knit and a poly(L-lactic acid) woven and a combination of a poly(glycolic acid) knit and a poly(glycolic acid) woven provide similar effects.

### Example 26

### Further Analysis of Support having a Double-layer Structure (Knit and Woven) of the Present Invention: Demonstration of Support for Cell Growth

Next, a double-layer support of the present invention was further analyzed in terms of cell growth activity.

<Cell Growth Experiment>

A support comprising a PLA or PGA knit and a PLGA woven which are adhered together with caprolactone was prepared. This support was used to examine an effect of type I collagen sponge crosslinking treatment on cell growth. Also, an effect of type IV collagen and laminin was investigated.

Specifically, a support comprising a PLA or PGA knit and a PLGA woven which are adhered together with caprolactone was subjected to: type I collagen sponge crosslinking treatment; type I+IV collagen sponge crosslinking treatment; and type I collagen+Laminin sponge crosslinking treatment. As a control, a support comprising a PGA knit and a PLGA woven which are adhered together with caprolactone was subjected to no crosslinking treatment. Rat vascular endothelial cells and smooth muscle cells were suspended in DMEM+20% FCS medium to 1×10⁵ cells/ml. 40 ml of the cell suspension was placed in a 100-ml Erlenmeyer flask. The above-described supports having a size of 1×1 cm were placed in the flask (per day, n=5). Dynamic culture (60 rpm) was conducted. The amount of cells accepted by the support was assessed by MTT assay on day 1,3 and 7.

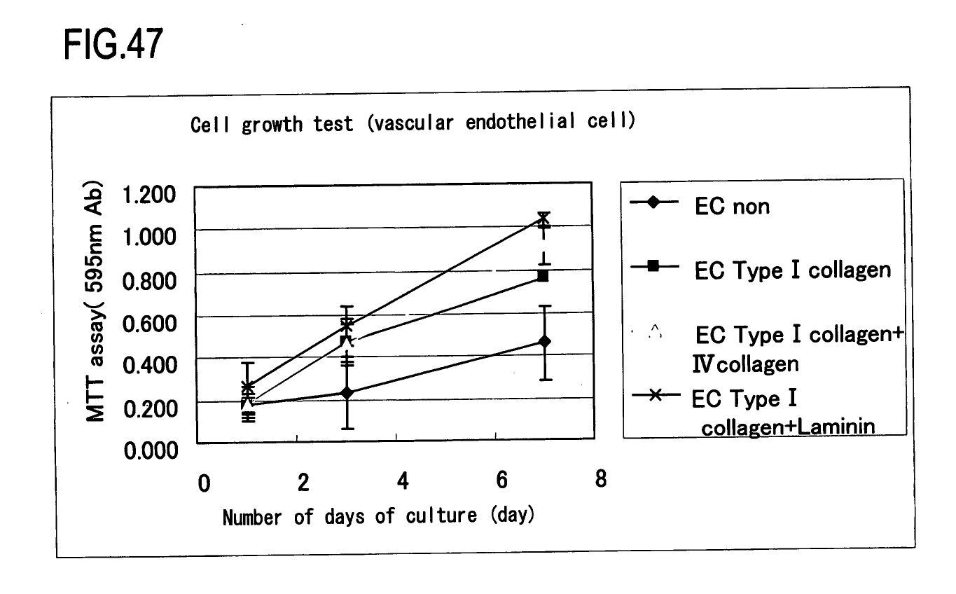

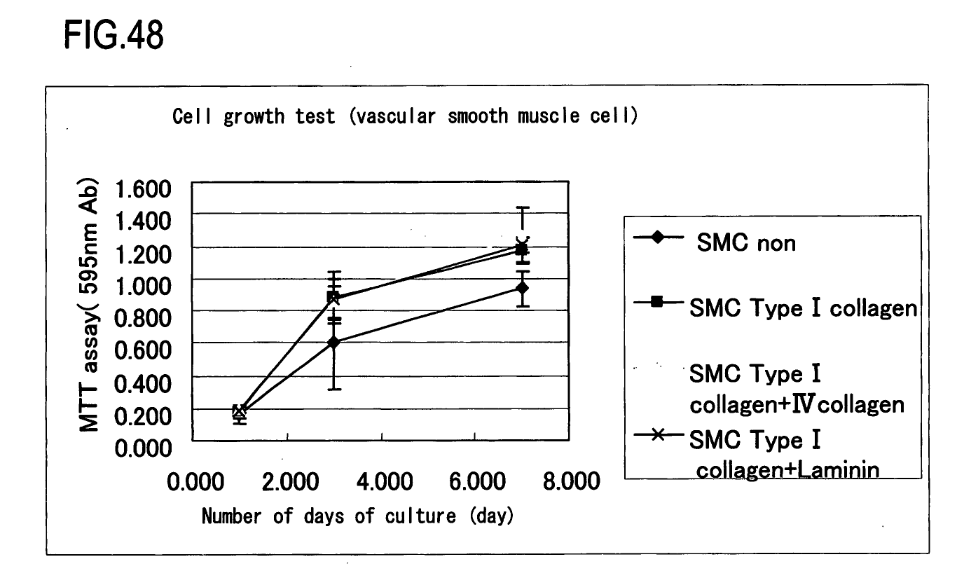

The results are shown in the table below. The results of the cell growth experiment are shown in FIG. 47 (vascular endothelial cell) and FIG. 48 (vascular smooth muscle cell).

| | MTT assay | | | | | |
|---|---|---|---|---|---|---|
| | Mean | | | S.D. | | |
| Cell growth test | 1 | 3 | 7 | 1 | 3 | 7 |
| EC non | 0.177 | 0.229 | 0.459 | 0.053 | 0.170 | 0.176 |
| EC Type I collagen | 0.177 | 0.470 | 0.762 | 0.037 | 0.113 | |
| EC Type I | 0.182 | 0.467 | 0.910 | 0.080 | 0.092 | 0.086 |
| collagen + IV | | | | | | |
| collagen | | | | | | |
| EC Type I | 0.265 | 0.549 | 1.033 | 0.116 | 0.094 | 0.028 |
| collagen + Laminin | | | | | | |
| SMC non | 0.165 | 0.599 | 0.934 | 0.055 | 0.284 | 0.107 |
| SMC Type I | 0.179 | 0.882 | 1.170 | 0.043 | 0.165 | 0.081 |
| collagen | | | | | | |
| SMC Type I | 0.200 | 0.855 | 1.269 | 0.023 | 0.101 | 0.169 |
| collagen + IV | | | | | | |
| collagen | | | | | | |
| SMC Type I | 0.190 | 0.873 | 1.211 | 0.013 | 0.127 | 0.047 |
| collagen + Laminin | | | | | | |

Type I collagen crosslinking treatment achieved a higher level of cell growth for both vascular endothelial cells and vascular smooth muscle cells, though the effect of type I collagen crosslinking treatment on cell adhesion was not considerably significant under the present experimental conditions. In this experiment, type I collagen crosslinking treatment supplemented with type IV collagen and laminin did not have a considerably significant effect.

The results above show a combination of a poly(glycolic acid) knit and a poly(L-lactic acid) woven. A similar effect was seen in the case of a combination of a poly(glycolic acid) knit and a poly(glycolic acid) woven. Poly(L-lactic acid) seems to be sometimes preferable since it is difficult to degrade. However, the present invention is not limited to poly(L-lactic acid). Rather, it should be noted that both the above-described combinations could achieve the object of the present invention.

### Example 27

### Effect of Other Cytokines on Another Animal

In Example 27, it is demonstrated that angiopoietin (R&D Systems); HGF (PeproTech, Inc.); FGF (fibroblast growth factor, trade name: Fibrast spray (Kaken Pharmaceutical); G-CSF (granulocyte colony stimulating factor, trade name: GRAN (Kirin Brewery); laminin (Becton, Dickinson and Company); SDF-1 (Decton, Dickinson and Company), TNF-α (Peprotech, Inc.), and IL1-β (Peprotech, Inc.) are used as biological molecules of the present invention and can act on the pulmonary artery and myocardial infarction site of dogs.

An experiment is conducted for the pulmonary artery and myocardial infarction site of dogs. Specifically, as in Examples 16 and 17, beagle dogs are used to demonstrate the effect of various cytokines on a support of the present invention.

The support implanted in the pulmonary artery has a smooth internal surface observed with the naked eye. HE staining indicated complete absorption of PGA and PLA and a tissue structure comparable to normal blood vessels.

The vascular endothelial cells are studied by Factor VIII staining and the vascular smooth muscle cells are studied by α-SMA immunostaining. α-SMA immunostaining is conducted using antibodies for α-SMA. The Factor VIII immunostaining indicates a monolayer of continuous vascular endothelial cells and the α-SMA immunostaining indicates the smooth muscle cells aligned on the internal surface.

Moreover, the vascular elastic fiber is studied by elastica van Gieson staining. Elastic fiber is observed in an internal layer of a blood vessel.

4 weeks after implantation, echocardiography is conducted for the myocardial infarction site. The ejection rate and the left ventricular fractional shortening are significantly improved in all cytokine treatment groups as compared to a control group. Such an improvement is retained until at least 8 weeks after implantation.

The cytokine treatment groups have a significant increase in the thickness of the LV wall and a significant reduction in the LV cross section as compared to the control group. The microscopic inspection reveals that a newly formed heart tissue compensates for a part of the LV wall suffering from infarction. In the support of the present invention, angiogenesis and the vanishment of the support (patch) are observed.

The results above show a combination of a poly(glycolic acid) knit and a poly(L-lactic acid) woven and a combination of a poly(glycolic acid) knit and a poly(glycolic acid) woven have substantially the same effect. Poly(L-lactic acid) seems to be sometimes preferable since it is difficult to degrade. However, the present invention is not limited to poly(L-lactic acid). Rather, it should be noted that both the above-described combinations could achieve the object of the present invention.

### Example 28

### Fray Test of Support with Double-Layer Structure (Knit and Woven) of the Present Invention

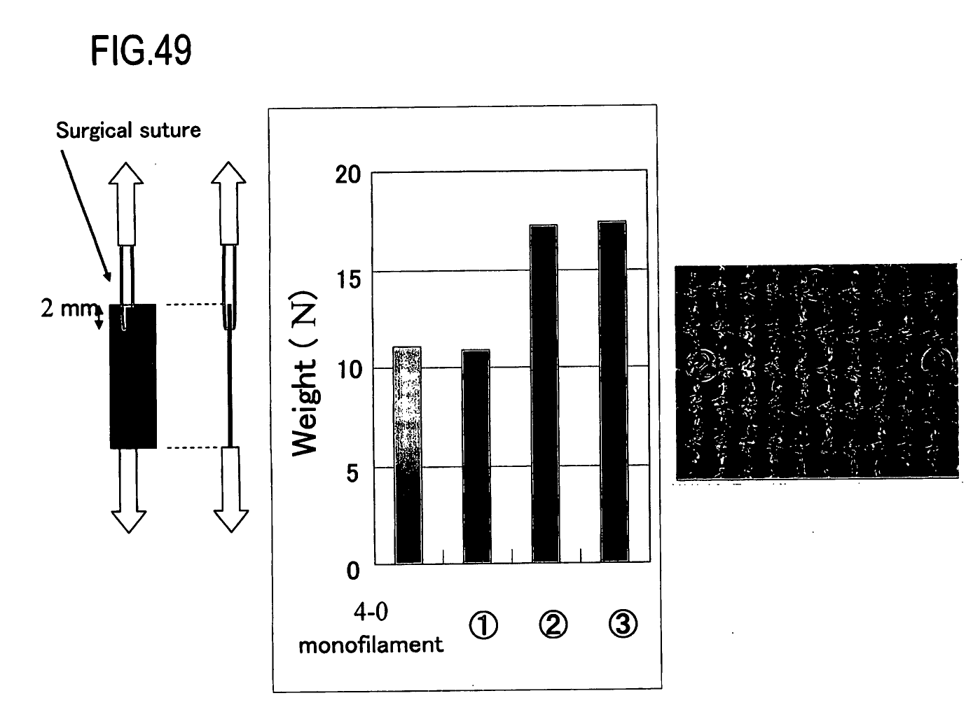

In Example 28, a fray test was conducted as shown in FIG. 49 so as to examine whether or not a double-layer support of the present invention is more difficult to fray.

A fray test was conducted as follows. A support of the present invention having a size of 1 cm×2 cm was prepared. A surgical suture was stitched into the support 2 mm below the top. The support was stretched vertically with a load. Fray resistance was represented by the weight of the load which the support could resist. The result is shown in FIG. 49, where fray resistance is indicated in three directions in comparison with monofilament. As can be seen from FIG. 49, fray resistance in the transverse direction was significantly increased by a factor of 2 or more.

### Example 29

### Implantation Experiment of Support having Double-Layer Structure (Knit and Woven) of the Present Invention

In Example 29, an experiment was conducted so as to demonstrate that a double-layer support of the present invention can be actually accepted in an organism for a long term.

A support of the present invention (poly(glycolic acid) (knit) and poly(L-lactic acid) (woven); 15 mm×10 mm) was implanted into the pulmonary artery or the aorta of adult beagle dogs (8 to 12 kg) under partial clamping. 2 weeks, 2 months or 6 months after implantation, the implanted site was histologically studied. The study was conducted using smooth muscle actin and (SMA) and Factor VIII as in the above-described examples.

<In Vivo: Two Weeks After Implantation>

No clear thrombus formation was observed in the implanted support with the naked eye. In the case of HE staining, residues of the support was observed and connective tissue was present therebetween.

<In Vivo: Two Months After Implantation>

The implanted support had a smooth internal surface observed with the naked eye. HE staining indicated complete absorption of PGA and PLA and a tissue structure comparable to normal blood vessels.

The vascular endothelial cells were studied by Factor VIII staining and the vascular smooth muscle cells were studied by α-SMA immunostaining. α-SMA immunostaining was conducted using antibodies for α-SMA. The Factor VIII immunostaining indicated a monolayer of continuous vascular endothelial cells and the α-SMA immunostaining indicated the smooth muscle cells aligned on the internal surface.

Moreover, the vascular elastic fiber was studied by elastica van Gieson staining. Elastic fiber was observed in an internal layer of a blood vessel.

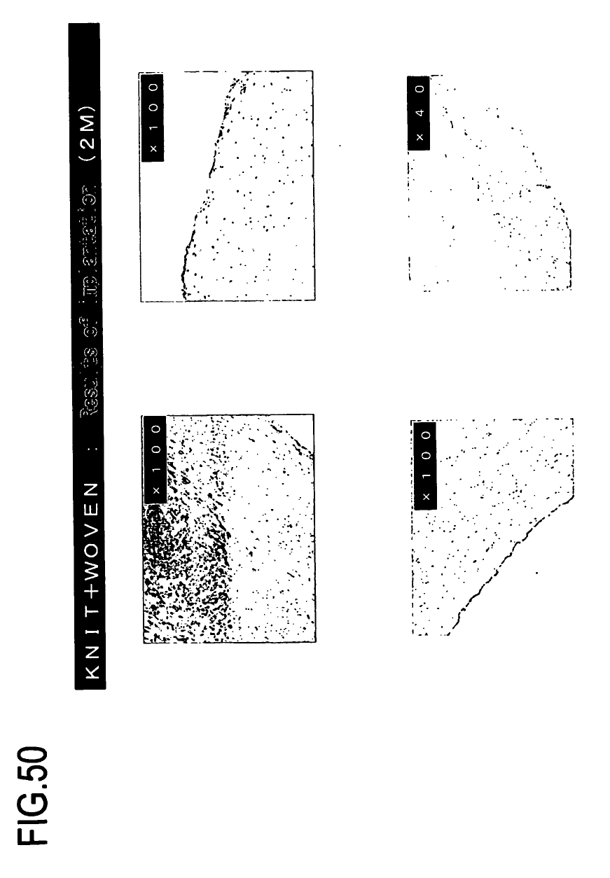

According to the SMA staining and the like, it was clarified that recellularization spread into the internal portion of the knit-woven support of the present invention (FIG. 50).

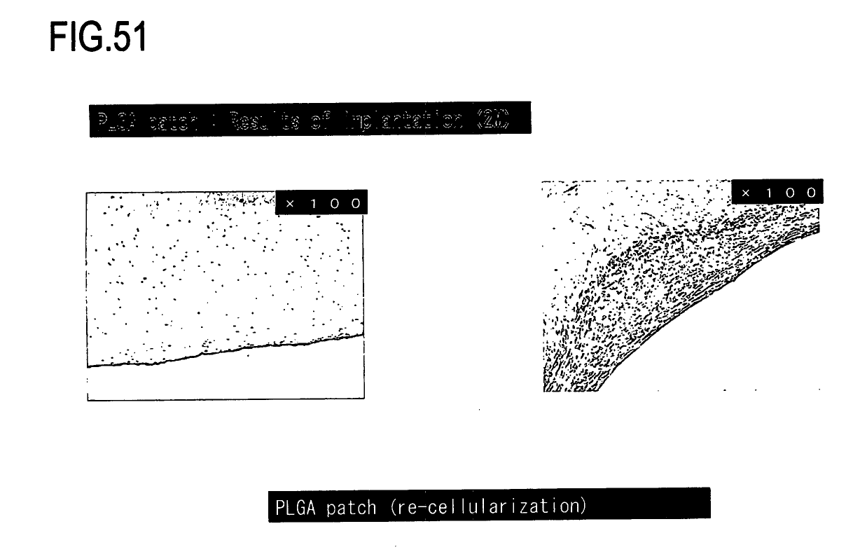

As compared to the PLGA copolymer support subjected to the same treatment (FIG. 51), the degree of recellularization was higher in the support having the above-described combination.

<In Vivo: Six Months After Implantation>

As observed two months after implantation, a monolayer of continuous vascular endothelial cells were observed by Factor VIII immunostaining. The morphology of the smooth muscle cells was clearly observed as compared to what was observed two months after implantation. α-SMA immunostaining indicated that the smooth muscle cells were aligned on the internal surface and had substantially the same morphology as in normal blood vessels. Elastica van Gieson staining indicated that a larger amount of vascular elastic fiber was observed in an internal layer of a blood vessel than at two months after implantation. The presence or absence of calcification in blood vessels was studied by von Kossa staining. A positive reaction was not observed in the implanted composite film and blood vessels in its vicinity, i.e., calcification was not observed.

### Example 30

### Patch with Monocusp

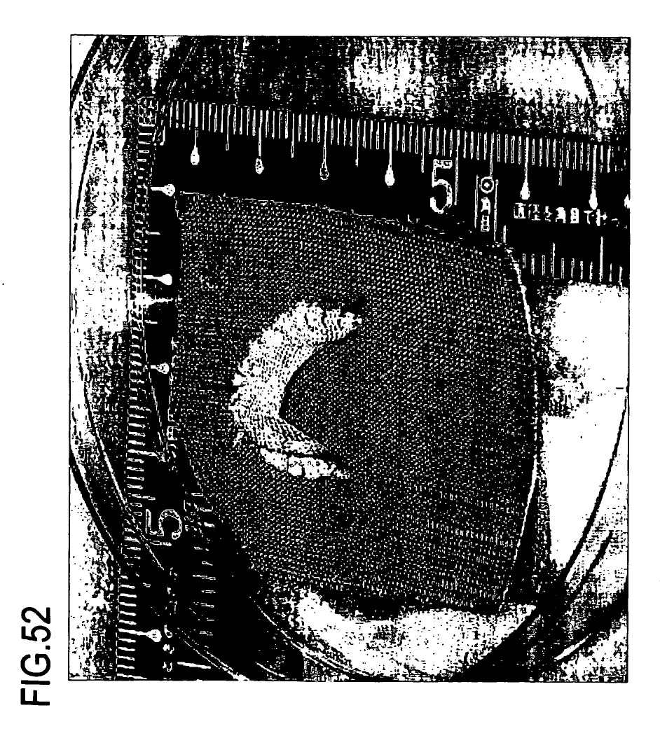

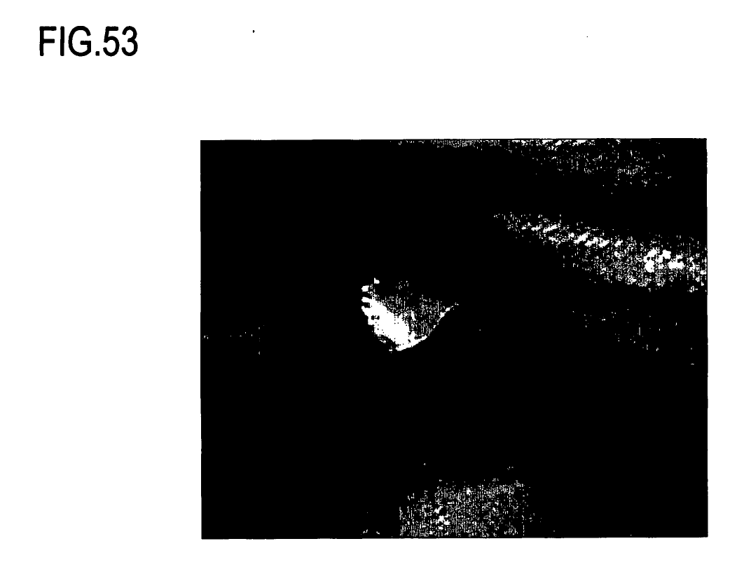

Next, a support having a cusp (monocusp patch) of the present invention was produced (FIGS. 52 and 53) as follows.

(Methods)

Type I collagen-microsponge and a biodegradable polymer of poly-lactic-co-glycolic acid (PLGA) were compounded to make the patch. The biodegradable scaffold reinforced with woven poly-lactic acid mesh cross-linking with collagen-microsponge was formed into a transannular patch with monocusp. This transannular patch was grafted onto the dog right ventricular outflow tract without pre-cellularization (n=3).

The details of the material and methods are descried below.

(Detailed Material and Methods)

(Scaffold Design)

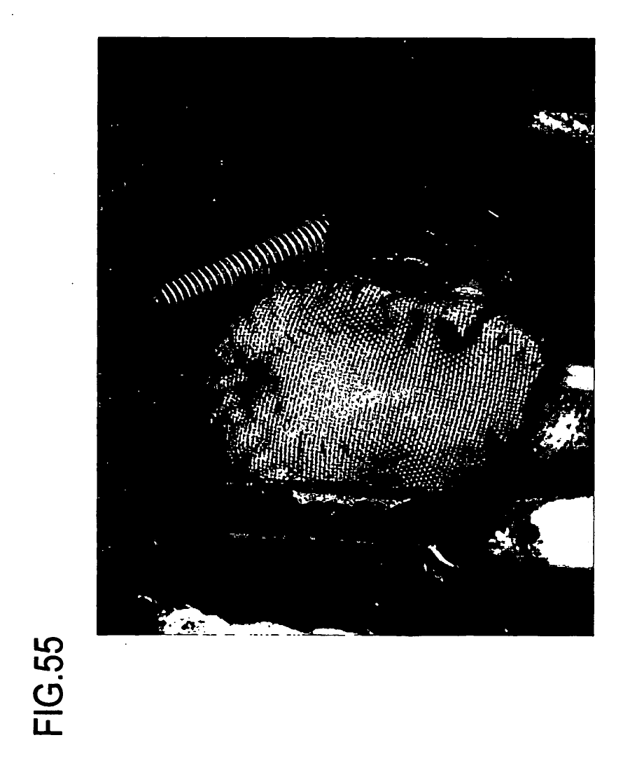

The biodegradable scaffold reinforced on the outside with woven poly-lactic acid (PLA) mesh cross-linking with collagen-microsponge was formed into a transannular patch with monocusp. The monocusp was also consisted of PLA woven (FIG. 55). These polymer scaffold provided from Senko Medical Instrument Mfg. Co., Ltd., (Osaka, Japan).

(In-Vivo Study)

The transannular patch with monocusp (50×30 mm) was grafted onto the mongrel dog (body weight 20 kg) right ventricular outflow tract (n=3). By means of femoral artery and right atrial venous cannulation, normothermic cardiopulmonary bypass was performed. With the heart beating, the pulmonary trunk to right ventricular outflow tract was incised longitudinally, and an anterior native leaflet was removed. The transannular patch without pre-cellularization was implanted using running 5-0 monofilament sutures. Transesophageal echocardiography (TEE) and angiography two months after grafting was examined about leaflet function and pulmonary regurgitation.

All animals received humane care in compliance with the Guide for the Care and Use of Laboratory Animals published by the National Institutes of Health as mentioned hereinabove.

(Results)

In the transannular patch model, echocardiography and angiography two months after grafting showed good leaflet function and no pulmonary regurgitation.

(In-Vivo Study)

FIG. 52 shows a state in which the monocusp patch of the present invention was actually implanted in an organism.

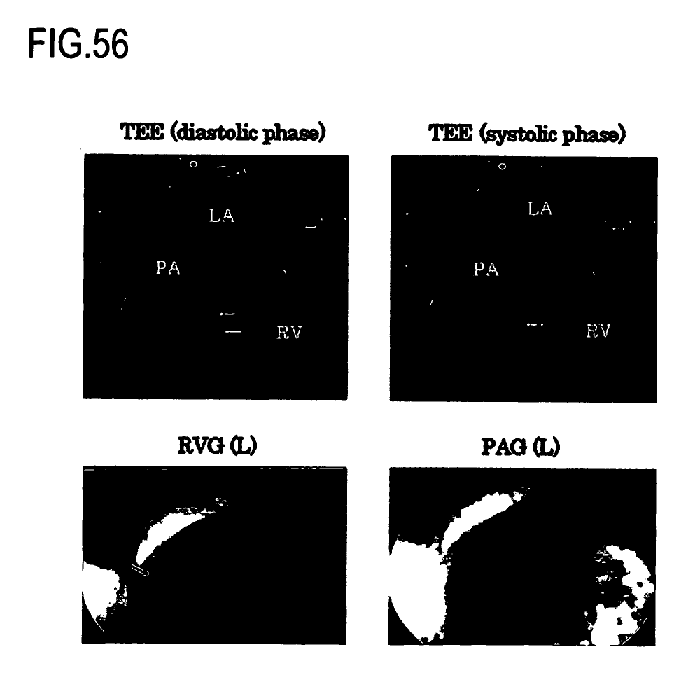

Echocardiography was conducted so as to determine whether or not the monocusp patch of the present invention actually functioned as a cusp. The result is shown in FIG. 56. In the cases with transannular patch, TEE and angiography two months after grafting showed no thrombus formation and right ventricular outflow tract stenosis. The synthetic leaflet has good functioning. There was no pulmonary regurgitation (FIG. 56).

As shown in FIG. 56, it was revealed that the monocusp support of the present invention in the middle of each figure actually functioned as a cusp.

In this example of transannular patch, TEE and angiography two months after grafting showed good leaflet functioning and no pulmonary regurgitation.

For the clinical applications, we improved the procedure of making the collagen-microsponge simply by using type I collagen alone.

In conclusion, the bioengineered graft made of biodegradable polymer and a biologically active agent (preferably microsponge type) showed comparable histological findings and durability even without pre-cellularization. This bioengineered graft is a promising surgical material for the in-situ cellularization which leads to a regeneration of autologous tissue in cardiovascular surgery.

FIG. 55 shows a state of a support with a monocusp of the present invention which was actually implanted. Echocardiography was used to determine whether or not the monocusp support of the present invention functioned as an actual cusp. The result is shown in FIG. 56. As can be seen from FIG. 56, it was clearly found that the cusp-like support of the present invention shown in the middle of the photographs functioned as an actual cusp.

Although certain preferred embodiments have been described herein, it is not intended that such embodiments be construed as limitations on the scope of the invention except as set forth in the appended claims. Various other modifications and equivalents will be apparent to and can be readily made by those skilled in the art, after reading the description herein, without departing from the scope and spirit of this invention. All patents, published patent applications and publications cited herein are incorporated by reference as if set forth fully herein.

### INDUSTRIAL APPLICABILITY

The present invention provides an implant capable of being cellularized without self-reproducing material derived from organisms, such as a cell. By implanting such an implant, an organ or tissue can be regenerated. The present invention is useful particularly for the regenerative medicine industry.

1. A biocompatible implant, comprising:
A) a biological molecule; and
B) a support, wherein the biological molecule is type I collagen.

2. (canceled)

3. (canceled)

4. (canceled)

5. (canceled)

6. (canceled)

7. (canceled)

8. (canceled)

9. (canceled)

10. (canceled)

11. A biocompatible implant according to claim 1, wherein the biological molecule further includes type IV collagen.

12. A biocompatible implant according to claim 1, wherein the biological molecule further includes a cytokine.

13. A biocampatible implant according to claim 1, wherein the support is in the form of a membrane.

14. A biocompatible implant according to claim 1, wherein the support is in the form of a tube.

15. A biocompatible implant according to claim 1, wherein the support is in the form of a valve.

16. A biocompatible implant according to claim 1, wherein the support includes biodegradable polymer.

17. A biocompatible implant according to claim 1, wherein the support includes at least one component selected from the group consisting of poly(glycolic acid) (PGA), poly(L-lactic acid) (PLA) and polycaprolactum (PCLA).

18. A biocompatible implant according to claim 1, wherein the support includes PGLA having a glycolic acid to lactic acid ratio of from about 90: about 10 to about 80: about 20.

19. A biocompatible implant according to claim 1, wherein the support includes a cell adhesion molecule.

20. A biocompatible implant according to claim 1, wherein the support includes a protein.

21. A biocompatible implant according to claim 1, wherein the support is in the form of a mesh and a sponge.

22. A biocompatible implant according to claim 1, wherein the support has a thickness of at least about 0.2 mm to about 1.0 mm.

23. A biocompatible implant according to claim 1, wherein the support has a strength of at least about 20 N.

24. A biocompatible implant according to item 1, wherein the support has a strength of at least about 50N.

25. A biocompatible implant according to claim 1, wherein the support is coated with the biological molecule.

26. A biocompatible implant according to claim 1, wherein the support has a gap and the gap is filled with the biological molecule.

27. A biocompatible implant according to claim 1, wherein the biological molecule and the support include a crosslinking molecule, and the crosslinking molecules are crosslinked between the support and the biological molecule.

28. A biocompatible implant according to claim 1, wherein the support includes the same material as the biological molecule.

29. A biocompatible implant according to claim 1, wherein a cell is attached to the biocompatible implant.

30. A biocompatible implant according to claim 1, for use in implantation into a body.

31. A biocompatible implant according to claim 30, wherein a site of the body 8into which the biologi8cal implant is implanted is selected from the group consisting of cardiac valve, blood vessel, pericardium, cardiac septum, intracardiac conduit, extracardiac conduit, duramater, skin, bone, soft tissue and trachea.

32. A biocompatible implant according to claim 1, which is sterilized.

33. A biocompatible implant according to claim 1, further comprising an immuno suppressant.

34. A biocompatible implant according to claim 1, further comprising an additional medicament component.

35. A biocompatible implant according to claim 30, wherein the biocompatible implant is derived from an organism undergoing the implantation.

36. A medicament according to claim 1, comprising a biocompatible implant according to claim 1.

37. A medical kit, comprising:
a biocompatible implant according to claim 1; and
instructions describing usage of the implant,
wherein the instructions describe that the implant is administered to a predetermined site.

38. A medical kit according to claim 37, wherein the predetermined site is selected from the group consisting of vascular endothelium, vascular smooth muscle, elastic fiber, skeletal muscle, cardiac muscle, osteoblast, neuron and collagen fiber.

39. A medical kit according to claim 37, wherein the instructions describe that the biocompatible implant is implanted in such a manner that at least a part of an organ or tissue to be subjected to implantation is left in situ.

40. A method for treating an injured site of a body, comprising the step of:
A) implanting a biocompatible implant to a part or whole of the injured site,
wherein the biocompatible implant comprises:
A-1) a biological molecule; and
A-2) a support, wherein the biological molecule is type I collagen.

41. A method according to claim 40, wherein in the implanting step, the biocompatible implant is implanted in such a manner that at least a part of an organ or tissue to which the injured site belongs is left in situ.

42. A method according to claim 40, further comprising administering a cellular physiologically active substance.

43. A method according to claim 42, wherein the cellular physiologically active substance is selected from the group consisting of a granulocyte macrophage colony stimulating factor (GM-CSF), a macrophage colony stimulating factor (M-CSF), a granulocyte colony stimulating factors (G-CSF), a multi-CSF (IL-3), a leukemia inhibiting factor (LIF), a c-kit ligand (SCF), an immunoglobulin family member, CD2, CD4, CD8, CD44, collagen, elastin, proteoglycan, glycosaminoglycan, fibronectin, laminin, syndecan, aggrecan, an integrin family member, integrin a chain, integrin β chain, fibronectin, laminin, vitronectin, selectin, cadherin, ICM1, ICAM2, VCAM1, platelet derived growth factor (PDGF), epidermal growth factor (EGF), fibroblast growth factor (FGF), hepatocyte growth factor (HGF) and vascular endothelial growth factor (VEGF), and polypeptides and peptides related thereto.

44. A method according to claim 40, further comprising performing a treatment for suppressing an immune reaction.

45. A method for reinforcing an organ or tissue in a body, comprising the step of:
A) implanting a biocompatible implant to a part or whole of the organ or tissue,
wherein the biocompatible implant comprises:
A-1) a biological molecule; and
A-2) a support, wherein the biological molecule is type I collagen.

46. A method for producing or regenerating an organ or tissue, comprising the steps of:
A) implanting a biocompatible implant to a part or whole of the organ or tissue within an organism containing the organ ox tissue,
wherein the biocompatible implant comprises:
A-1) a biological molecule; and
A-2) a support, wherein the biological molecule is type I collagen; and
B) culturing the organ or tissue within the organism.

47. Use of a biocompatible implant according to claim 1 for treatment of an injured site within a body.

48. Use of a biocompatible implant according to claim 1 for reinforcement of an organ or tissue within a body.

49. Else of a biocompatible implant according to claim 1 for production of a medicament for treatment of an injured site within a body.

50. Use of a biocompatible implant according to claim 1 for production of a medicament for reinforcement of an organ or tissue within a body.

51. A biocompatible tissue support, comprising;
A) a first layer having a rough surface; and
B) a second layer having a strength which allows the second layer to resist in vivo impact,
wherein the first layer is attached to the second layer via at least one point, wherein the first layer is a knit, and wherein the second layer is a woven.

52. (canceled)

53. (canceled)

54. A support according to claim 51, wherein the rough surface has sufficient space for accommodating cells.

55. A support according to claim 51, wherein the attachment is carried out by melting a biological absorbable macromolecule.

56. A support according to claim 51, wherein the second layer has substantially no permeability to air.

57. A support according to claim 51, wherein the strength of the support is at least 100 N.

58. A support according to claim 51, wherein the air permeability of the support is no more than 10 ml/cm²/sec.

59. A support according to claim 51, wherein the first layer includes a biodegradable material.

60. A support according to claim 51, wherein the first layer includes at least one component selected from the group consisting of poly(glycolic acid) (PGA), poly(L-lactic acid) (PLA), and polycaprolactum (PCLA) and a copolymer thereof.

61. A support according to claim 51, wherein the first layer includes PGLA having a glycolic acid-to-lactic acid ratio of about 90: about 10 to about 80: about 20.

62. A support according to claim 51, wherein the second layer includes poly(glycolic acid).

63. A support according to claim 51, wherein the second layer includes a biodegradable material.

64. A support according to claim 51, wherein the second layer includes at least one component selected from the group consisting of poly(glycolic acid) (PGA), poly(L lactic acid)(pLA) and polycaprolactum (PCLA), and a copolymer thereof.

65. A support according to claim 51, wherein the second layer includes PGLA having a glycolic acid-to-lactic acid ratio of from about 90: about 10 to about 80: about 20.

66. A support according to claim 51, wherein the second layer includes poly(L-lactic acid).

67. (canceled)

68. A support according to claim 51, wherein the second layer is a woven of poly(L-lactic acid) and the first layer is a knit of poly(glycolic acid).

69. A support according to claim 51, wherein the attachment is carried out by:
C) an intermediate layer for attaching the first layer with the second layer.

70. A support according to claim 69, wherein the intermediate layer is made of a synthetic biological absorbable polymer.

71. A support according to claim 69, wherein the intermediate layer includes a homopolymer containing a single monomer selected from the group consisting of lactic acid (lactid), glycolide and ε-caprolactam or a copolymer containing two or more monomers therefrom.

72. A support according to claim 69, wherein the intermediate layer includes a material having a melting point lower than a melting point of the second layer and a melting point of the first layer.

73. A support according to claim 51, wherein the first layer comprises a plurality of knit layers.

74. A support according to claim 51, wherein the first layer comprises a plurality of knit layers.

75. A support according to claim 51, wherein a biological molecule is provided on the first layer.

76. A support according to claim 75, wherein the biological molecule is an extracellular matrix.

77. A support according to claim 75, wherein the biological molecule includes an extracellular matrix selected from the group consisting of collagen and laminin.

78. A support according to claim 75, wherein the biological molecule is contained in a microsponge and the microsponge is provided on the first layer.

79. A support according to claim 75, wherein the biological molecule is crosslinked with the support.

80. A medical device comprising a support according to claim 51.

81. A medical device according to claim 80, further comprising a cell.

82. A medicament according to claim 80, for use in implantation into a body.

83. A medicament according to claim 80, wherein a site of the body into which the biological implant is implanted is selected from the group consisting of cardiac valve, blood vessel, pericardium, cardiac septum, intracardiac conduit, extracardiac conduit, dura mater, skin, bone, soft tissue anal trachea.

84. A medicament according to claim 80, wherein the biocompatible implant is derived from an organism undergoing the implantation.

85. A method for producing a biocompatible tissue support, wherein the biocompatible tissue support comprises:
A) a first layer having a rough surface; and
B) a second layer having a strength which allows the second layer to resist in vivo impact,
wherein the first layer is attached to the second layer via at least one point, wherein the first layer is a knit, and wherein the second layer is a woven, and
the method comprises the step of:
attaching the first layer with the second layer.

86. A method according to claim 85, wherein the biocompatible tissue support further comprises:
C) an intermediate layer for attaching the first layer with the second layer,
the attaching step comprises:
a) providing the intermediate layer between the first layer and the second layer;
b) providing the first layer, the second layer and the intermediate layer under conditions that the first layer and the second layer are not melted and the intermediate layer is melted; and
c) the intermediate layer is provided under conditions that the intermediate layer is solidified, while retaining desired shapes of the first layer, the second layer and the intermediate layer.

87. A method according to claim 86, wherein the melting point of the intermediate layer is lower than both the melting points of the first layer and the second layer and a difference between the melting points is utilized.

88. A method according to claim 86, wherein the second layer is a woven of poly(L-lactic acid) and the first layer is a kit of poly(glycolic acid), and the intermediate layer includes a homopolymer containing a single monomer selected from the group consisting of lactic acid (lactid), glycolide and ε-caprolactam or a copolymer containing two or more monomers therefrom.

89. A method according claim 88, wherein the temperature is higher than the melting point of the intermediate layer and is lower than the melting points of the first layer and the second layer.

90. A method according to claim 86, wherein the support furthe4r comprises a biological molecule and the method further comprises the step of:
attaching the biological molecule to the first layer.

91. A method according to claim 90, wherein the attaching step comprises crosslinking treatment.

92. A method according to claim 90, wherein the biological molecule is collagen, and the attaching step comprises collagen crosslinking treatment.

93. A method according to claim 86, wherein the intermediate layer is produced by casting a film material onto a glass plate, followed by air drying; to form a film.

94. A method according to claim 86, wherein the step b) comprises exerting a pressure of at least about 0.1 g/cm² onto the support.

95. A method according to claim 86, wherein the step b) comprises exerting a pressure of at least about 0.5 g/cm² onto the support.

96. A method for treating an injured site of a body, comprising the step of:
A) implanting a biocompatible tissue support to a part or whole of the injured site,
wherein the biocompatible tissue support comprises:
A-1) a first layer having a rough surface; and
A-2) a second layer having a strength which allows the second layer to resist in vivo impact,
wherein the first layer is attached to the second layer via at least one point wherein, the first layer is a knit, and wherein the second layer is a woven.

97. A method fox reinforcing an organ or tissue within a body, comprising the step of:
A) implanting a biocompatible tissue support to a part or whole of the inured site,
wherein the biocompatible tissue support comprises:
A-1) a first layer having a rough surface; and
A-2) a second layer having a strength which allows the second layer to resist in vivo impact,
wherein the first layer is attached to the second layer via at least one point wherein the first layer is a knit, and wherein the second layer is a woven.

98. A method for producing or regenerating an organ or tissue, comprising the steps of:
A) implanting a, biocompatible tissue support to a part or whole of the organ or tissue within an organism containing the organ or tissue,
wherein the biocompatible tissue support comprises:
A-1) a first layer having a rough surface; and
A-2) a second layer having a strength which allows the second layer to resist in vivo impact,
wherein the first layer is attached to the second layer via at least one point wherein the first layer is a knit, and wherein the second layer is a woven; and
B) culturing the organ or tissue in the organism.

99. Use of a biocompatible tissue support for treatment of an injured site within a body, wherein
the biocompatible tissue support comprises:
A-1) a first layer having a rough surface; and
A-2) a second layer having a strength which allows the second layer to resist in vivo impact,
wherein the first layer is attached to the second layer via at least one point wherein the first layer is a knit, and wherein the second layer is a woven.

100. Use of a biocompatible tissue support for reinforcement of an organ or tissue within a body, wherein the biocompatible tissue support comprises:
A-1) a first layer having a rough surface; and
A-2) a second layer having a strength which allows the second layer to resist in vivo impact,
wherein the first layer is attached to the second layer via at least one point wherein the first layer is a knit, and wherein the second layer is a woven.

101. Use of a biocompatible tissue support for production of a medicament for treatment of an injured site within a body, wherein
the biocompatible tissue support comprises:
A-1) a first layer having a rough surface; and
A-2) a second layer having a strength which allows the second layer to resist, in vivo impact,
wherein the first layer is attached to the second layer via at least one point wherein the first layer is a knit, and wherein the second layer is a woven.

102. Use of a biocompatible tissue support for production of a medicament for reinforcement of an organ or tissue within a body, wherein
the biocompatible tissue support comprises:
A-1) a first layer having a rough surface; and
A-2) a second layer having a strength which allows the second layer to resist in vivo impact,
wherein the first layer is attached to the second layer via at least one point wherein the first layer is a knit, and wherein the second layer is a woven.


SEQUENCE LISTING (10 sequences)

SEQ ID NO: 1 - protein, 7 aa, Artificial
  short peptide
SVVYGLR

SEQ ID NO: 2 - DNA, 23 bp, Artificial
  primer
accctggaattgctgatcgtatg

SEQ ID NO: 3 - DNA, 24 bp, Artificial
  primer
tgtcgtcctgagtgtaaggtagcc

SEQ ID NO: 4 - DNA, 24 bp, Artificial
  probe
aaattaccgcactggctcccagca

SEQ ID NO: 5 - DNA, 22 bp, Artificial
  primer
tagaatagcctcagaggcccag

SEQ ID NO: 6 - DNA, 20 bp, Artificial
  primer
gcttccgagaccgctctgtc

SEQ ID NO: 7 - DNA, 25 bp, Artificial
  probe
cagtccgtgccaatgacgacctgaa

SEQ ID NO: 8 - DNA, 22 bp, Artificial
  primer
tgctgaaggacactcaaatcca

SEQ ID NO: 9 - DNA, 22 bp, Artificial
  primer
gttgatgaggctggtgttctgg

SEQ ID NO: 10 - DNA, 24 bp, Artificial
  probe
acgcagtccgtgccaatgacgacc